(19) United States
(12) Patent Application Publication
(10) Pub. No.: US 2026/0207551 A1
(43) Pub. Date: Jul. 23, 2026

(54) PHOSPHOGLICERATE DEHYDROGENASE INHIBITORS FOR THE TREATMENT OF FIBROSIS

(71) Applicant: CHIESI FARMACEUTICI S.P.A., Parma (IT)

(72) Inventors: Adam FRIEDMAN, Parma (IT); Vipin SURI, Parma (IT)

(73) Assignee: CHIESI FARMACEUTICI S.P.A., Parma (IT)

(21) Appl. No.: 19/140,034

(22) PCT Filed: Dec. 13, 2023
(86) PCT No.: PCT/EP2023/085570

§ 371 Date: Jun. 17, 2025

Related U.S. Application Data
(60) Provisional application No. 63/433,504, filed on Dec. 19, 2022.

(51) Int. Cl. A61K 31/404; A61K 31/407; A61K 31/4178; A61K 31/422; A61K 31/4439; A61K 31/454; A61K 31/497; A61P 11/00

(52) U.S. Cl. CPC A61K 31/404; A61K 31/407; A61K 31/4178; A61K 31/422; A61K 31/4439; A61K 31/454; A61K 31/497; A61P 11/00

(57) ABSTRACT

The present invention relates to a new therapeutic use of compounds of PHGDH inhibitors of general formula (I), for the prevention and/or treatment of fibrosis, in particular idiopathic pulmonary fibrosis (IPF). The invention is related also the use of the pharmaceutical compositions and combinations comprising said compounds for the prevention and/or treatment of fibrosis and in particular IPF.

### FIELD OF INVENTION

The present invention relates to the use of compounds of general formula (I) as sole agent or in combinations with other active ingredients, as well as to the use of pharmaceutical compositions and combinations comprising said compounds for the prevention and/or treatment of fibrosis, in particular idiopathic pulmonary fibrosis (IPF).

### BACKGROUND OF THE INVENTION

Fibrosis is a pathological wound healing in which connective tissue replaces normal parenchymal tissue to the extent that it goes unchecked, leading to considerable tissue remodeling and the formation of permanent scar tissue (see Wynn, Thomas A., 2004 Nature Reviews 4 (8): 583-594)

Fibrosis can occur in many tissues within the body, typically as a result of inflammation or damage, and examples include liver, lungs, kidney, brain and heart.

Idiopathic pulmonary fibrosis (IPF) is a rare, progressive illness of the respiratory system, characterized by the thickening and stiffening of lung tissue, associated with the formation of scar tissue. It is a type of chronic scarring lung disease characterized by a progressive and irreversible decline in lung function. (see HR, Egan J J, et al. American Journal of Respiratory and Critical Care Medicine, 2011, 183 (6): 788-824.)

About 3 million people are affected globally and seems to become more common over the year, the incidence is expected to double by 2030 (see Fernandex Perez E R, et al, Chest, 2020; 137:129-137). Mortality is comparable to lung cancer, the median survival is 2-3 years after diagnosis, and respiratory failure is the most common cause of death (see Strongman H, et a., Adv Ther. 2018; 35: 724-736)

The currently two FDA-approved drugs on the market, Nintedanib and Pirfenidone, slow the progression of the disease and prolong the life expectancy of IPF patients; however, the treated patients continue to experience lung function loss and premature death and the adverse events of both Nintedanib and Pirfenidone critically affect the patient's quality of life.

The IPF is therefore a high unmet medical need and many efforts have been done in the recent past years to develop alternative treatments.

3-phosphoglycerate dehydrogenase (PHGDH) is the enzyme that catalyzes the first rate-limiting step in the de novo biosynthesis of serine starting from glucose, and it is considered as a major enzyme in the diversion of glycolysis towards serine synthesis. Serine, a critical amino acid for protein and nucleic acid biosynthesis, in turn is metabolized and incorporated into a variety of biomolecules including glycine (see Yang M, Vousden, K H., Nature Reviews Cancer 2016, 16, 650-662).

PHGDH is required to promote collagen protein synthesis. Indeed, glycine accounts for one-third of all amino acids within the collagen molecule, and the high content of glycine is critical for the stabilization of collagen helix.

Collagen is the main structural protein in the extracellular space, and it is produced in excess in IPF patients during fibroblasts into myofibroblasts differentiation that accumulates within the fibrotic tissue and leads to loss of organ architecture and function.

Moreover, PHGDH and the serine/glycine synthesis pathway is part of a wider network which links glycolysis with one-carbon metabolism and nucleotide synthesis contributing to cell proliferation in pathologies such as inflammation and fibrosis.

Therefore, the PHGDH inhibition has the potential to reduce the aberrant production and release of collagen such as inhibit cell proliferation (see Selvarajah et al., Science Signaling, 2019; 12(582):eaav3048).

Various compounds have been described in the literature as PHGDH inhibitors.

WO 2017156165 (Raze Therapeutics) discloses the compound of general formula (I) effective as PHGDH inhibitors, and their use in the treatment of many PHGDH-mediated disorder, in particular melanoma, breast, or lung cancer.

Alternative PHGDH inhibitors promising for the prevention and/or treatment of fibrosis, are described as noncompetitive inhibitors or allosteric inhibitors of PHGDH enzyme (see Pacold et al, Nat Chem Biol. 2016; 12: 452-458).

Examples of said PHGDH inhibitors useful for treating fibrotic diseases are disclosed in WO2016115463 (Whitehead Institute For Biomedical Research; Dana-Farber Cancer Institute, Inc).

In particular one compound, identified as NCT-503, has been tested in a bleomycin-induced Pulmonary Fibrosis model (see Hamanaka et al., Am J Respir Cel/Mol Biol. 2018 May; 58(5):585-593) showing a preliminary efficacy in attenuation of lung fibrosis in treated mice 7 days after intratracheal instillation of bleomycin.

Despite the above prior art, there remains a potential for developing medicaments comprising a PHGDH inhibitor useful for the prevention and/or treatment of fibrosis, in particular IPF, with an improved efficacy profile and at the same time characterized by a lower brain exposure to reduce the potential neurological adverse events.

The underlying problem of the present invention therefore lies in the provision of a medicament comprising a compound of formula (I) for the prevention and/or treatment of fibrosis, in particular IPF.

### SUMMARY OF THE INVENTION

In a first aspect the invention refers to a compound of Formula (I), or a pharmaceutically acceptable salt thereof, for use in the prevention and/or treatment of fibrosis,

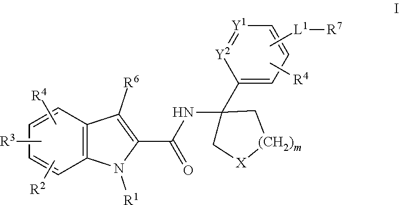

wherein
R¹ is hydrogen or C₁₋₄ alkyl;
each of R² and R³ is independently halogen, —OR, —CN, C₁₋₆ aliphatic optionally substituted with 1, 2, or 3 halogens, or -L-R⁸; or R² and R³ are optionally taken together with the carbon atoms to which they are attached and any intervening atoms to form a 5-8 membered partially unsaturated ring having 0-2 heteroatoms independently selected from nitrogen, oxygen, or sulfur;
each L is independently a C₁₋₆ bivalent straight or branched hydrocarbon chain wherein 1-4 methylene units of the chain are independently and optionally replaced with —O—, —C(O)—, —C(O)O—, —OC(O)—, —OC(O)N(R)—, —(R)NC(O)O—, —C(O)N(R)—, —(R)NC(O)—, —N(R)—, —N(R)C(O)N(R)—, —S—, —SO—, or —SO₂—;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
R⁸ is hydrogen, C₁₋₆ aliphatic, or an optionally substituted 4-8 membered saturated or partially unsaturated monocyclic heterocyclic ring having 1-3 heteroatoms independently selected from nitrogen, oxygen, or sulfur;
R⁴ is hydrogen, halogen, —OR⁵, —CN, C₁₋₆ aliphatic optionally substituted with 1, 2, or 3 halogens, or -L-R⁸
R⁵ is hydrogen, —(CH₂)ₙ-phenyl, —(CH₂)ₙ-Cy′, or C₁₋₆ alkyl optionally substituted with 1, 2, or 3 halogens;
each -Cy′- is a 4-8 membered saturated or partially unsaturated monocyclic heterocyclic ring having 1-2 heteroatoms independently selected from nitrogen, oxygen, or sulfur,
R⁶ is hydrogen or C₁₋₄ alkyl;
R⁷ is hydrogen, —CO₂R, optionally substituted C₁₋₆ aliphatic, -Cy-, or a bivalent 3-7 membered ring;
L¹ is a covalent bond or a C₁₋₈ bivalent straight or branched hydrocarbon chain wherein 1-5 methylene units of the chain are independently and optionally replaced with —O—, —C(O)—, —C(O)O—, —OC(O)—, —N(R)—, —C(O)N(R)—, —(R)NC(O), —OC(O)N(R), —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R), or -Cy-;
each -Cy- is independently a bivalent 6-membered arylene ring containing 0-2 nitrogen atoms, or a bivalent 5-membered heteroarylene ring with 1-4 heteroatoms independently selected from nitrogen, oxygen, or sulfur, or a bivalent partially unsaturated 8-10 membered bicyclic heterocyclene ring with 1-4 heteroatoms independently selected from nitrogen, oxygen, or sulfur, wherein -Cy- is optionally substituted with 1 or 2 substituents independently selected from C₁₋₄ alkyl or —OR;
X is O, S, or —N(R¹⁰)—;
R¹⁰ is C₁₋₆ aliphatic optionally substituted with 1, 2, or 3 halogens, —C(O)CH₃, or —SO₂—N(R¹)(R¹¹);
R¹¹ is —C(O)CH₃, —C(O)NHR¹, or pyrazinyl;
n is independently 0, 1, 2, 3, 4, or 5;
m is independently 0, 1, or 2; and
each of Y¹ and Y² is independently ═N— or ═C(R⁴)—;
or a pharmaceutically acceptable salt thereof.

In a second aspect, the invention refers to a compound of formula (I) or a pharmaceutically acceptable salt for use in the prevention and/or treatment of IPF.

In a third aspect, the invention refers to a pharmaceutical composition comprising a compound of formula (I) or a pharmaceutically acceptable salt thereof in admixture with one or more pharmaceutically acceptable carrier or excipient, for use in the prevention and/or treatment of fibrosis.

In a fourth aspect, the invention refers to a pharmaceutical composition comprising a compound of formula (I) or a pharmaceutically acceptable salt thereof in admixture with one or more pharmaceutically acceptable carrier or excipient, for use in the prevention and/or treatment of IPF.

### BRIEF DESCRIPTION OF THE FIGURES

FIG. 1: Inhibition of collagen expression

FIG. 2: Inhibition of α-SMA expression

FIG. 3: Inhibition of cell proliferation

FIG. 4: Plasma and tissue concentrations of Compound 19

FIG. 5: Inhibition of the synthesis of ¹³C-labelled serine in plasma for Compound 19

FIG. 6: Inhibition of the synthesis of ¹³C-labelled serine in lung for Compound 19

FIG. 7: Inhibition of the synthesis of ¹³C-labelled serine in brain for Compound 19

FIG. 8: Inhibition of the synthesis of ¹³C-labelled serine in plasma for Compound 19a

FIG. 9: Inhibition of the synthesis of ¹³C-labelled serine in lung for Compound 19a

FIG. 10: Inhibition of the synthesis of ¹³C-labelled serine in brain for Compound 19a

FIG. 11: Ashcroft score analysis for Compound 19

FIG. 12: Automated histological analysis (Artificial Intelligence APP—fibrotic area) for Compound 19

FIG. 13: Automated histological analysis (Artificial Intelligence APP—Collagen APP) for Compound 19

FIG. 14: Markers in Lung homogenates for Compound 19

FIG. 15: Plasma Serine level for Compound 19

FIG. 16: Plasma levels of Compound 19 after the last dose

FIG. 17: Efficacy on FVC decline compared to Saline group for Compound 19a

FIG. 18: Biomarkers in BALF (A and B) and in Plasma (C) for Compound 19a

FIG. 19: Ashcroft score analysis and automated analysis of fibrosis for Compound 19a

FIG. 20: Ashcroft score analysis for NCT-503

FIG. 21: Markers in Lung homogenates for NCT-503

FIG. 22: Plasma Serine level for NCT-503

FIG. 23: Plasma and brain levels of NCT-503 after the last dose

### DETAILED DESCRIPTION OF THE INVENTION

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or a substitutable nitrogen of a heterocyclic ring, for example N (as in 3,4-dihydro-2H-pyrrolyl), NH (as in pyrrolidinyl) or NR* (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

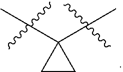 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms, having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzothiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or *NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent.

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

Unless otherwise stated, structures depicted herein are also meant to include all isomeric (e.g., enantiomeric, diastereomeric, and geometric (or conformational)) forms of the structure, for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms.

The term “pharmaceutically acceptable salts”, as used herein, refers to derivatives of compounds wherein the parent compound is suitably modified by converting any of the free acid or basic group, if present, into the corresponding addition salt with any base or acid conventionally intended as being pharmaceutically acceptable.

The term “fibrotic disease” or “fibrosis” of “fibrotic conditions” refers to conditions that are associated with the abnormal accumulation of cells and/or fibronectin and/or collagen and/or increased fibroblast recruitment and include but are not limited to pulmonary fibrosis, familiar pulmonary fibrosis, idiopathic pulmonary fibrosis (IPF), chronic hypersensitivity pneumonitis (CHP), hepatic fibrosis, kidney or renal fibrosis, ocular fibrosis, cardiac fibrosis, arterial fibrosis, sarcoidosis and systemic sclerosis, liver fibrosis or Cirrhosis.

The term “PHGDH” or “phosphoglycerate dehydrogenase”, is alternatively referred to as 3-PGDH, 3PGDHm HEL-S-113, NLS, PDG, PGAD, PGD, PGDH, PHGDHD, or SERA. The term “PHGDH” encompasses mutants, variants, homologs, fragments, and synthetically modified phosphoglycerate dehydrogenases.

The term “treating”, or “treatment” of a disease state includes: (i) inhibiting the disease state, i.e. arresting the development of the disease state or its clinical symptoms, or (ii) relieving the disease state, i.e. causing temporary or permanent regression of the disease state or its clinical symptoms.

The term “preventing”, or “prevention” of a disease state includes causing the clinical symptoms of the disease state not to develop in a subject that may be exposed to or predisposed to the disease state but does not yet experience or display symptoms of the disease state. For example, treating or preventing a respiratory disease or disorder includes treating or preventing the symptoms the disorder such as cough and/or urge to cough associated with a respiratory disease.

The term “therapeutically effective amount” means an amount of a compound that, when administered to a subject for treating a disease state, is sufficient to affect such treatment for the disease state. The “therapeutically effective amount” will vary depending on the compound, disease state being treated, the severity or the disease treated, the age and relative health of the subject, the route, and form of administration.

The present invention relates to the compounds of general formula (I) as herein defined for the use in prevention and/or treatment of fibrosis.

Differently from the other PHGDH inhibitors of the prior art, such as NCT-503, the compounds of formula (I) are surprisingly efficacious in the treatment of fibrosis, in particular IPF, in a substantive and effective way, particularly appreciated by the skilled person when looking at improved treatment for fibrosis.

As indicated in the Experimental part, the compounds of formula (I) have shown an improved in vitro profile and demonstrated an antifibrotic activity in in vitro and in vivo model.

The antifibrotic activity of preferred compounds of formula (I) has been monitored measuring the expression of fibrotic mediators, collagen I (Col-I) and alpha smooth muscle actin (α-SMA).

As can be appreciated in Example 1 in Table 2, the Compound 19 is able to reduce the fibroblast to myofibroblast transition (FMT) induced by TGF-β and to inhibit the release of α-SMA in NHLF treated with TGF-β (10 ng/ml) for 48 h and differently from the compound NCT-503, the Compound 19 is also able to reduce the cell proliferation induced by serum treatment in NHLFs (72 h).

The antifibrotic activity of compounds of formula (I) has been demonstrated also measuring the expression collagen I (Col-I), known fibrotic mediator, in Fibroblast to myofibroblast transition (FMT) in in vitro assay. Furthermore, collagen is a downstream effector of the PHGDH pathway, its inhibition underlines the key role of this marker in fibrosis processes. These findings suggest that de novo synthesis of serine and glycine is required for pulmonary fibrosis; the inhibition of myofibroblast differentiation and collagen deposition is strongly correlated and necessary for the antifibrotic activity of the compounds.

As reported in the Table 3 in Example 2, all other tested compounds, dose-dependently, reduce the expression of aSMA, after 72 hours of incubation, differently from the NCT-503 which partially reduces the expression of aSMA. Furthermore, tested compounds dose-dependently inhibit the collagen I deposition, after 72 h of treatment.

More advantageously, the compounds of formula (I) show a surprising lower CNS (Central nervous system) exposure than the prior at compound NCT-503. As reported in Example 3, thanks to the low brain exposure, the risk of potential neurological adverse effects already known for other PHGDH inhibitors is significantly reduced.

As further advantage, the compounds of the present invention are resulted to be particularly efficacious also in the most applied in vivo model, the Bleomycin-induced pulmonary fibrosis in mice. Bleomycin-induced pulmonary fibrosis in mice is the most commonly applied in vivo experimental model to induce lung fibrosis. Instillation of Bleomycin in the trachea induce a multiphasic response that starts with an acute and severe inflammation followed by a diffuse matrix and collagen deposition that led to histological changes, with evident fibrosis accumulation and loss of functional parenchymal tissue, replicating certain pathological features consistent with those observed in the lungs of IPF patients.

Indeed, the bleomycin animal model is widely used in the assessment of potential antifibrotic agents.

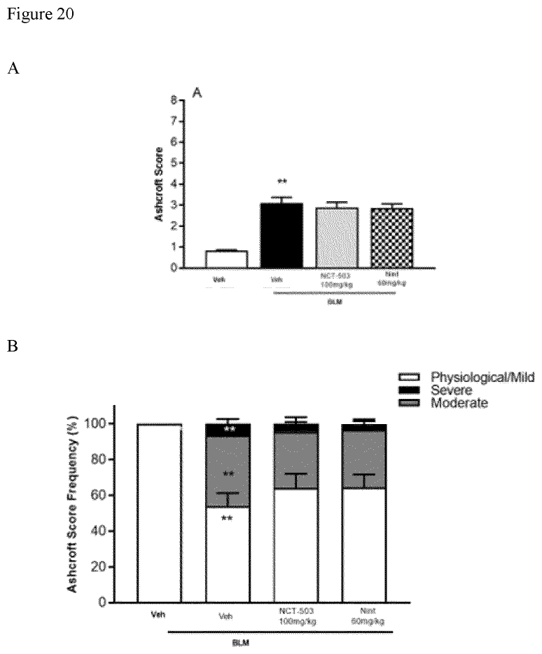

As can be appreciated in Example 5 and represented in FIGS. 11A, 11B, 12 and 13, the Compound 19, significantly reduces the pathological features of the induced fibrosis measured by the Ashcroft score and by an Automated histological analysis; on the contrary, as reported in FIG. 20, the compound NCT-503 does not diminish neither the Bleomycin-induced lung fibrosis nor the fibrosis severity.

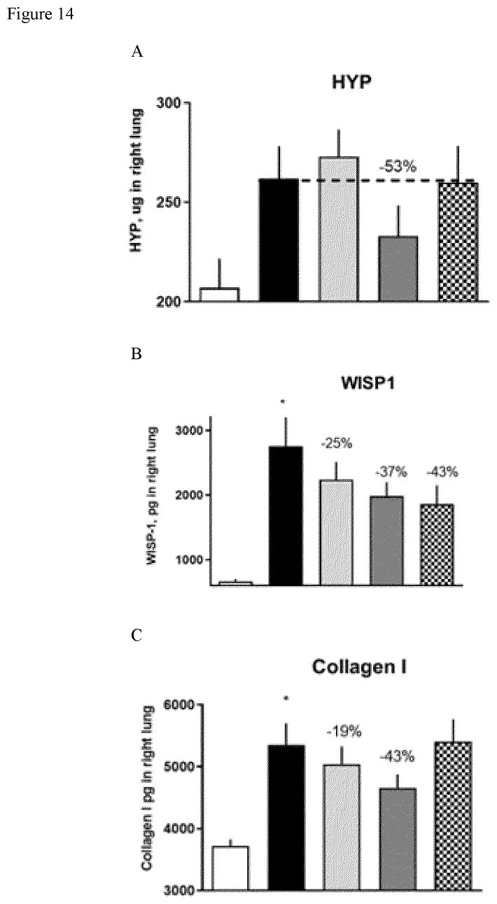

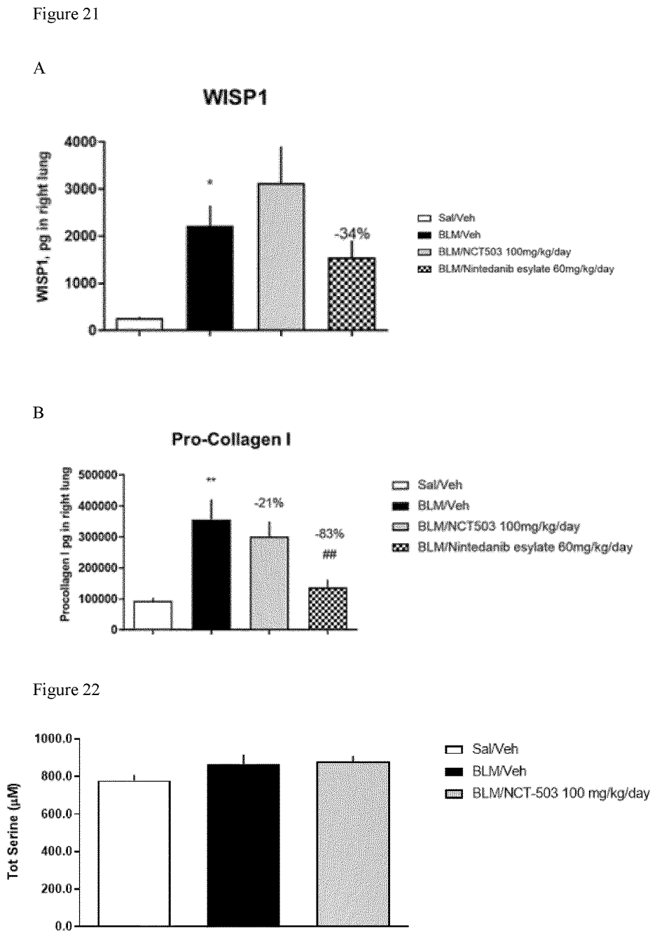

Moreover, as additional proof of an improved efficacy, the compounds of the present invention are able to reduce the level of fibrosis markers typically induced by the bleomycin treatment such as collagen accumulation (pro-CollagenI) and matrix deposition (WISP-1), as can be appreciated in FIGS. 14A, 14B and 14C. Quite the contrary, as shown in comparative Example 7 and in FIGS. 21A and 21B, the treatment with NCT-503 does not affect at all the levels of the same markers.

As can be appreciated in Example 6 and represented in FIG. 19A for the Compound 19a, one diastereoisomer of Compound 19, in the Bleomycin model Ashcroft score showed a significant increase in fibrosis, which was markedly reduced after treatment with Compound 19a. As shown in FIG. 19B, the Compound 19a was able to reduce in a dose dependent manner the fibrotic tissue getting an evident effect.

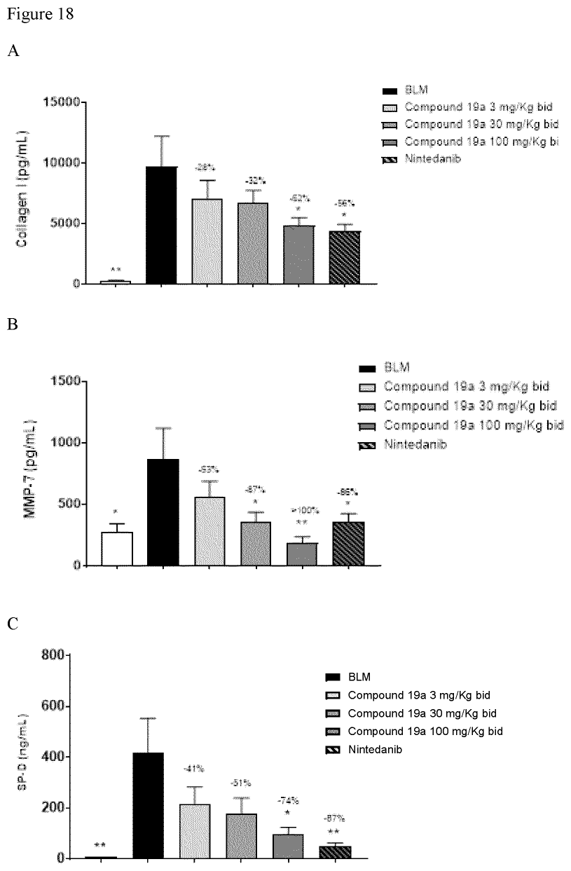

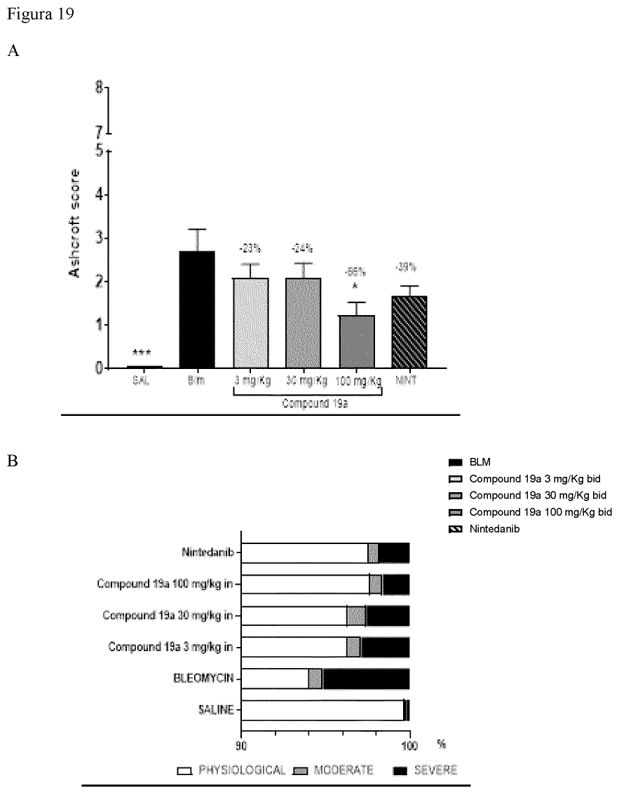

The bleomycin instillation induced a significant increase in lung levels of Collagen I and MMP-7 evaluated in BALF (FIGS. 18A and 18B) and an improved by 77% the FVC (Forced Vital Capacity, a lung function parameter) decline at the dose of 100 mg/kg. The content of these biomarkers was significantly reduced in a dose dependent manner by Compound 19a, getting the maximum effect at dose even lower to Compound 19.

Accordingly, the compounds of formula (I) demonstrated efficacy in the treatment of fibrosis, and in particular treatment of idiopathic pulmonary fibrosis, whenever PHGDH receptors are involved.

In one embodiment, the invention relates to the compounds of formula (I) or a pharmaceutically acceptable salt thereof, for use in the prevention and/or treatment of fibrosis

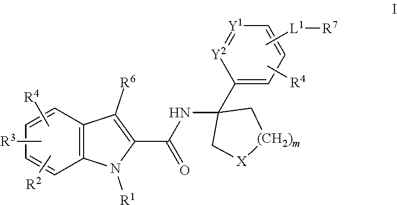

wherein:
R¹ is hydrogen or C₁₋₄ alkyl;
each of R² and R³ is independently halogen, —OR, —CN, C₁₋₆ aliphatic optionally substituted with 1, 2, or 3 halogens, or -L-R⁸; or R² and R³ are optionally taken together with the carbon atoms to which they are attached and any intervening atoms to form a 5-8 membered partially unsaturated ring having 0-2 heteroatoms independently selected from nitrogen, oxygen, or sulfur;
each L is independently a C₁₋₆ bivalent straight or branched hydrocarbon chain wherein 1-4 methylene units of the chain are independently and optionally replaced with —O—, —C(O)—, —C(O)O—, —OC(O)—, —OC(O)N(R)—, —(R)NC(O)O—, —C(O)N(R)—, —(R)NC(O)—, —N(R)—, —N(R)C(O)N(R)—, —S—, —SO—, or —SO₂—;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
R⁸ is hydrogen, C₁₋₆ aliphatic, or an optionally substituted 4-8 membered saturated or partially unsaturated monocyclic heterocyclic ring having 1-3 heteroatoms independently selected from nitrogen, oxygen, or sulfur;
R⁴ is hydrogen, halogen, —OR⁵, —CN, C₁₋₆ aliphatic optionally substituted with 1, 2, or 3 halogens, or -L-R⁸.
R⁵ is hydrogen, —(CH₂)~-phenyl, —(CH₂)ₙ-Cy′, or C₁₋₆ alkyl optionally substituted with 1, 2, or 3 halogens;
each -Cy′- is a 4-8 membered saturated or partially unsaturated monocyclic heterocyclic ring having 1-2 heteroatoms independently selected from nitrogen, oxygen, or sulfur,
R⁶ is hydrogen or C₁₋₄ alkyl;
R⁷ is hydrogen, —CO₂R, optionally substituted C₁₋₆ aliphatic, -Cy-, or a bivalent 3-7 membered ring;
L¹ is a covalent bond or a C₁₋₈ bivalent straight or branched hydrocarbon chain wherein 1-5 methylene units of the chain are independently and optionally replaced with —O—, —C(O)—, —C(O)O—, —OC(O)—, —N(R)—, —C(O)N(R)—, —(R)NC(O), —OC(O)N(R), —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R), or -Cy-;
each -Cy- is independently a bivalent 6-membered arylene ring containing 0-2 nitrogen atoms, or a bivalent 5-membered heteroarylene ring with 1-4 heteroatoms independently selected from nitrogen, oxygen, or sulfur, or a bivalent partially unsaturated 8-10 membered bicyclic heterocyclene ring with 1-4 heteroatoms independently selected from nitrogen, oxygen, or sulfur, wherein -Cy- is optionally substituted with 1 or 2 substituents independently selected from C₁₋₄ alkyl or —OR;
X is O, S, or —N(R¹⁰)—;
R¹⁰ is C₁₋₆ aliphatic optionally substituted with 1, 2, or 3 halogens, —C(O)CH₃, or —SO₂—N(R¹)(R¹¹);
R¹¹ is —C(O)CH₃, —C(O)NHR¹, or pyrazinyl;
n is independently 0, 1, 2, 3, 4, or 5;
m is independently 0, 1, or 2; and
each of Y¹ and Y² is independently ═N— or ═C(R⁴)—;
or a pharmaceutically acceptable salt thereof.

All the listed groups for each of the variable moieties R, R¹, R², R³, R⁴, R⁵, R⁶, R⁷, R⁸, L, L¹, R¹⁰, R¹¹, X, n, m, Cy, Cy′, Y¹ and Y² of the compounds of the invention have to be intended as alternatives and may be combined with each other in embodiments which are included in the scope of the invention.

In one embodiment, R¹ is hydrogen or C₁₋₄ alkyl. In one preferred embodiment, R¹ is methyl or ethyl. In a more preferred embodiment, R¹ is methyl.

In one embodiment, each of R² and R³ is independently halogen, —OR, —CN, C₁₋₆ aliphatic optionally substituted with 1, 2, or 3 halogens, or -L-R⁸; or R² and R³ are optionally taken together with the carbon atoms to which they are attached and any intervening atoms to form a 5-8 membered partially unsaturated ring having 0-2 heteroatoms independently selected from nitrogen, oxygen, or sulfur.

In one embodiment, R² is selected from the group consisting of halogen, —OR, -L-R⁸,

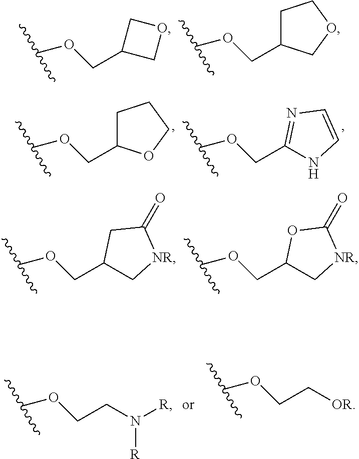

In one preferred embodiment, R² is selected from the group consisting of F, Cl, —CF₃, —OCF₃, —OCHF₂, —OCH₂Ph, —OCH₃, —CN, —CH₃,

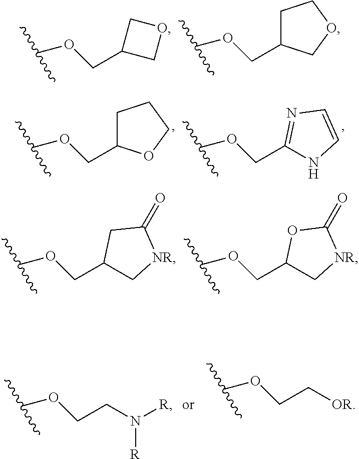

In a more preferred embodiment, R² is selected from the group consisting of F, Cl, —OCH₃ and —CH₃.

In one embodiment, R³ is selected from the group consisting of halogen, OR,

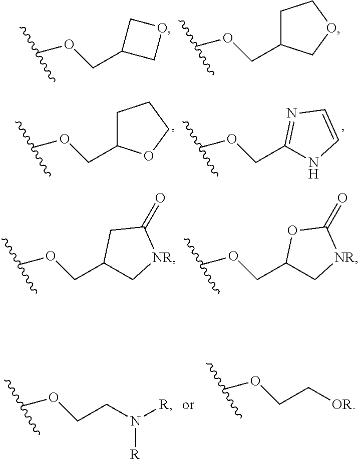

In one preferred embodiment, R³ is selected from the group consisting of F, Cl, —OCH₃ and —CH₃. In a more preferred embodiment, R³ is —CH₃.

In one embodiment, R⁴ is hydrogen, halogen, —OR⁵, —CN, C₁₋₆ aliphatic optionally substituted with 1, 2, or 3 halogens, or -L-R⁸. In a preferred embodiment, R⁴ is selected from the group consisting of H, halogen, —OR,

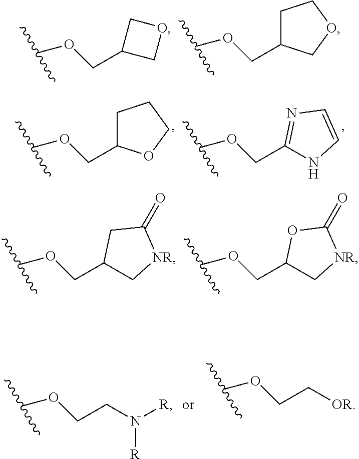

In one preferred embodiment, R⁴ is selected from the group consisting of hydrogen, F, Cl, —OCH₃ and —CH₃. In a more preferred embodiment, R⁴ is hydrogen.

In one embodiment, R⁵ is hydrogen, —(CH₂)~-phenyl, —(CH₂)ₙ-Cy′, or C₁₋₆ alkyl optionally substituted with 1, 2, or 3 halogens. In a preferred embodiment R⁵ is hydrogen or C₁₋₆ alkyl optionally substituted with 1, 2, or 3 halogens.

In one embodiment, R⁶ is hydrogen or C₁₋₄ alkyl. In a preferred embodiment, R⁶ is hydrogen or methyl.

In one embodiment, R⁸ is independently hydrogen, C₁₋₆ aliphatic, or an optionally substituted 4-8 membered saturated or partially unsaturated monocyclic heterocyclic ring having 1-3 heteroatoms independently selected from nitrogen, oxygen, or sulfur. In a preferred embodiment R⁸ is independently hydrogen or C₁₋₆ aliphatic.

In one embodiment, R is selected from the group comprising hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In a preferred embodiment, R is hydrogen.

In one embodiment, R⁶ is hydrogen or C₁₋₄ alkyl. In a preferred embodiment, R⁶ is hydrogen or methyl.

In one embodiment L¹ is a covalent bond or a C₁₋₈ bivalent straight or branched hydrocarbon chain wherein 1-5 methylene units of the chain are independently and optionally replaced with —O—, —C(O)—, —C(O)O—, —OC(O)—, —N(R)—, —C(O)N(R)—, —(R)NC(O), —OC(O)N(R), —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R), or -Cy-. In a preferred embodiment, L¹ is a C₁₋₈ bivalent straight or branched hydrocarbon chain wherein 1-5 methylene units of the chain are independently and optionally replaced with —O—, —C(O)—, —C(O)O—, —OC(O)—, —N(R)—, —C(O)N(R)—, —(R)NC(O), —OC(O)N(R), —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R), or -Cy-.

In a preferred embodiment, L¹ is selected from the group consisting of SO₂NH—,

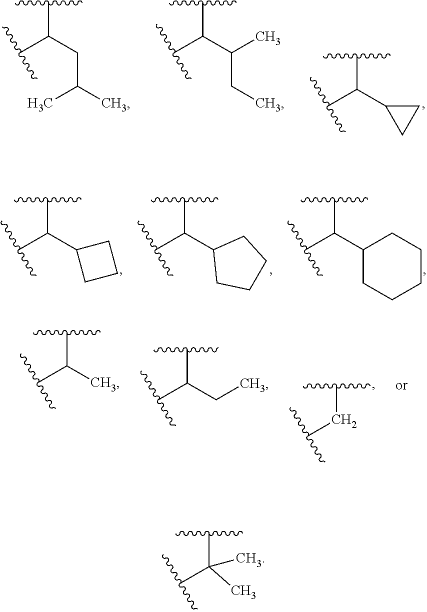

In a more preferred embodiment, L¹ is selected from the group consisting of

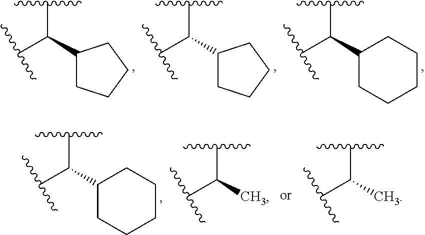

In one embodiment, R⁷ is selected from the group consisting of hydrogen, —CO₂R, optionally substituted C₁₋₆ aliphatic, -Cy-, or a bivalent 3-7 membered ring. In one preferred embodiment, R⁷ is selected from the group consisting of hydrogen, methyl, ethyl, cyclopropyl, cyclobutyl, tetrazolyl, and —CO₂H.

In one embodiment, each -Cy- is independently a bivalent 6-membered arylene ring containing 0-2 nitrogen atoms, or a bivalent 5-membered heteroarylene ring with 1-4 heteroatoms independently selected from nitrogen, oxygen, or sulfur, or a bivalent partially unsaturated 8-10 membered bicyclic heterocyclene ring with 1-4 heteroatoms independently selected from nitrogen, oxygen, or sulfur, wherein -Cy- is optionally substituted with 1 or 2 substituents independently selected from C₁₋₄ alkyl or —OR.

In one embodiment, R¹⁰ is C₁₋₆ aliphatic optionally substituted with 1, 2, or 3 halogens, —C(O)CH₃, or —SO₂—N(R¹)(R¹¹). In a preferred embodiment, R¹⁰ is C₁₋₆ aliphatic, optionally substituted with 1, 2, or 3 halogens, or —C(O)CH₃.

In one embodiment, X is O, S, or —N(R¹⁰)—. In one preferred embodiment, X is O.

In one embodiment, each of Y¹ and Y² is independently ═N— or ═C(R⁴)—. In a preferred embodiment, Y¹ and Y² are —CH.

All the listed groups for each of the variable moieties R, R¹, R², R³, R⁴, R⁵, R⁶, R⁷, R⁸, L, L¹, R¹⁰, R¹¹, X, n, m, Cy, Cy′, Y¹ and Y² of the compounds of the invention have to be intended as alternatives and may be combined with each other in embodiments which are included in the scope of the invention.

In another embodiment the invention relates to the compounds for formula (I) as above defined for use in the prevention and/or treatment of fibrosis selected from the group consisting of pulmonary fibrosis, familiar pulmonary fibrosis, idiopathic pulmonary fibrosis (IPF), chronic hypersensitivity pneumonitis (CHP), hepatic fibrosis, kidney or renal fibrosis, ocular fibrosis, cardiac fibrosis, arterial fibrosis, sarcoidosis and systemic sclerosis, liver fibrosis and Cirrhosis.

In one preferred embodiment, the invention relates to the compounds of formula (I) as above defined for use in the prevention and/or treatment of IPF.

In an equally preferred embodiment, the invention relates to the compound of formula (I) as above defined for use in the prevention and/or treatment of renal fibrosis.

In some embodiments, the present invention provides a compound of Formulae II-a, II-b, II-c, II-d, or II-e for use in prevention and/or treatment of fibrosis,

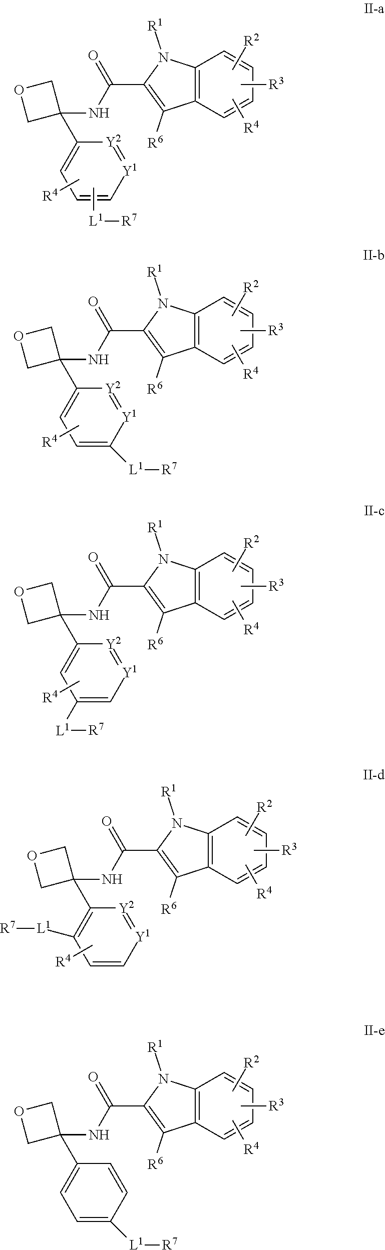

or a pharmaceutically acceptable salt thereof, wherein each of R¹, R², R³, R⁴, R⁶, R⁷, L¹, Y¹ and Y² are defined above and described in embodiments herein.

In one preferred embodiment, the present invention relates to a compound which is selected from Formulae II-a, II-b, II-c, II-d, or II-e as above defined for use in prevention and/or treatment of fibrosis selected from the group consisting of pulmonary fibrosis, familiar pulmonary fibrosis, idiopathic pulmonary fibrosis (IPF), chronic hypersensitivity pneumonitis (CHP), hepatic fibrosis, kidney or renal fibrosis, ocular fibrosis, cardiac fibrosis, arterial fibrosis, sarcoidosis and systemic sclerosis, liver fibrosis and Cirrhosis.

In a still preferred embodiment, the present invention relates to a compound which is selected from Formulae II-a, II-b, II-c, II-d, or II-e as above defined for use in prevention and/or treatment of IPF.

In an equally preferred embodiment, the present invention relates to a compound which is selected from Formulae II-a, II-b, II-c, II-d, or II-e as above defined for use in prevention and/or treatment of renal fibrosis.

In one embodiment, the present invention provides a compound which is selected from Formulae III-a, III-b, III-c, III-d, III-e, ITT-f, III-g, III-h, or III-i for use in the prevention and/or treatment of fibrosis:

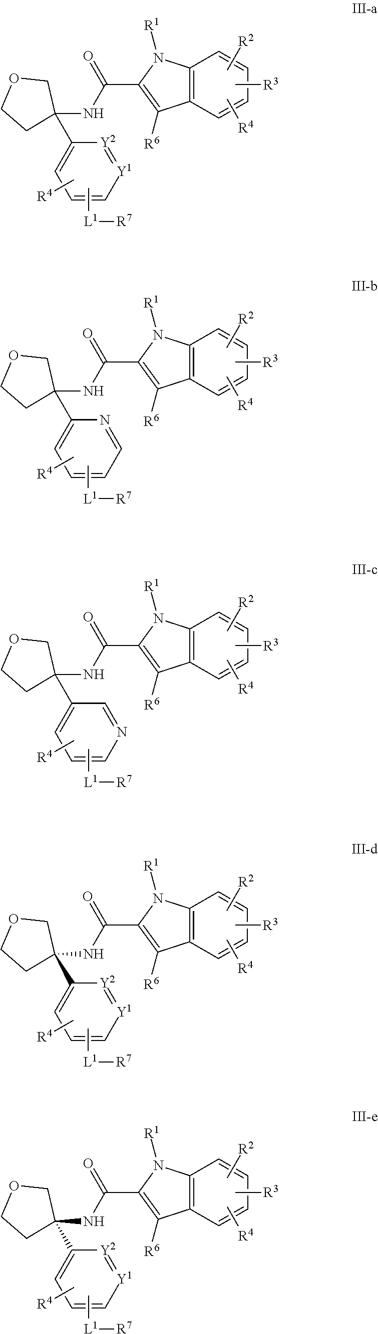

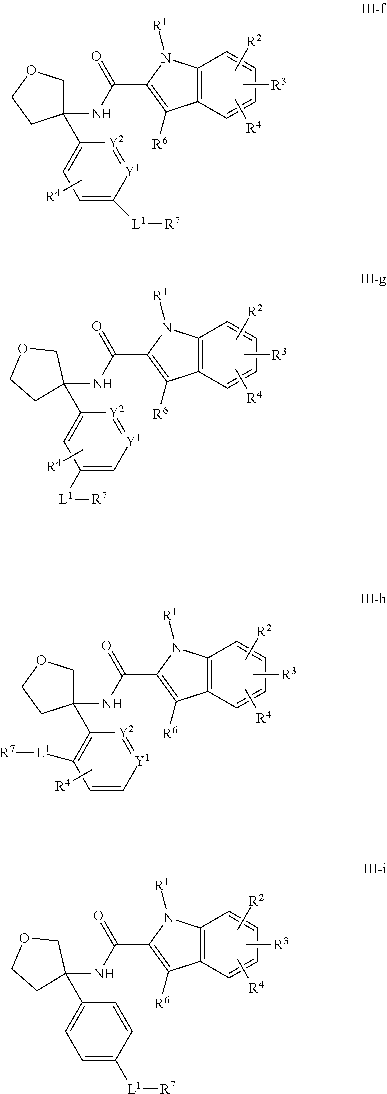

or a pharmaceutically acceptable salt thereof, wherein each of R¹, R², R³, R⁴, R⁶, R⁷, L¹, Y¹ and Y² are defined above and described in embodiments herein.

In one preferred embodiment, the present invention provides a compound which is selected from Formulae III-a, III-b, III-c, III-d, III-e, III-f, III-g, III-h, or III-i as above defined for use in the prevention and/or treatment of fibrosis selected from the group consisting of pulmonary fibrosis, familiar pulmonary fibrosis, idiopathic pulmonary fibrosis (IPF), chronic hypersensitivity pneumonitis (ClIP), hepatic fibrosis, kidney or renal fibrosis, ocular fibrosis, cardiac fibrosis, arterial fibrosis, sarcoidosis and systemic sclerosis, liver fibrosis and Cirrhosis.

In one preferred embodiment, the present invention provides a compound which is selected from Formulae ITT-a, ITT-b, ITT-c, TTT-d, III-e, ITT-f, TTT-g, ITT-h, or ITT-i as above defined for use in the prevention and/or treatment of IPF.

In an equally preferred embodiment, the present invention provides a compound which is selected from Formulae ITT-a, ITT-b, ITT-c, TTT-d, III-e, ITT-f, TTT-g, ITT-h, or ITT-i as above defined for use in the prevention and/or treatment of renal fibrosis.

According to preferred embodiments, the present invention relates to at least one of the compounds selected from those listed in the following Table 1 for use in the prevention and/or treatment of fibrosis:

TABLE 1
List of preferred compounds of Formula (I)

| Compound | | |
|---|---|---|
| No. | Chemical Name | Structure |
| 1 | N-(3-(4-(N- Acetylsulfamoyl)phenyl) oxetan-3-yl)-4,5-dichloro-1- methyl-1H-indole-2- carboxamide | 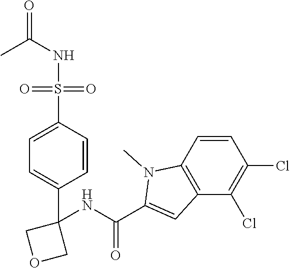 [SMILES: CC(=O)NS(=O)(=O)c1ccc(C2(NC(=O)c3cc4c(Cl)c(Cl)ccc4n3C)COC2)cc1] |
| 2 | (±)-3-{4-[3-(4,5-dichloro-1- methyl-1H-indole-2- amido)oxetan-3- yl]phenyl}oxolane-3- carboxylic acid | 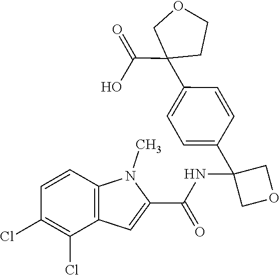 [SMILES: Cn1c(C(=O)NC2(c3ccc(C4(C(=O)O)CCOC4)cc3)COC2)cc2c(Cl)c(Cl)ccc21] |
| 3 | (±)-3-cyclopropyl-2-{4-[3- (4,5-dichloro-1-methyl-1H- indole-2-amido)oxetan-3- yl]phenyl}propanoic acid | 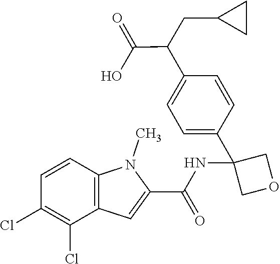 [SMILES: Cn1c(C(=O)NC2(c3ccc(C(CC4CC4)C(=O)O)cc3)COC2)cc2c(Cl)c(Cl)ccc21] |
| 4 | (±)-3-cyclopropyl-2-{4-[3- (4,5-dichloro-6-methoxy-1- methyl-1H-indole-2- amido)oxetan-3- yl]phenyl}propanoic acid | 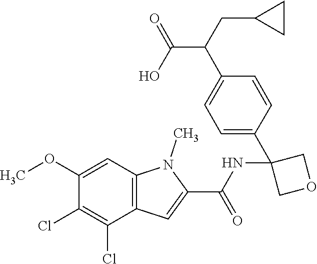 [SMILES: COc1cc2c(cc(C(=O)NC3(c4ccc(C(CC5CC5)C(=O)O)cc4)COC3)n2C)c(Cl)c1Cl] |
| 5 | 2R)-2-[4-[3-[(4-chloro-5- methoxy-1-methyl-indole-2- carbonyl)amino]oxetan-3- yl]phenyl]-2-cyclopentyl- acetic acid | 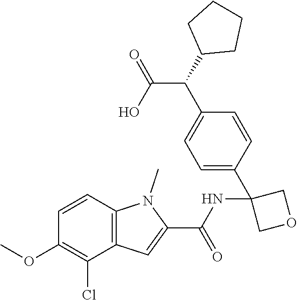 [SMILES: COc1ccc2c(cc(C(=O)NC3(c4ccc([C@H](C(=O)O)C5CCCC5)cc4)COC3)n2C)c1Cl] |
| 6 | (2S)-2-[4-[3-[(4-chloro-5- methoxy-1-methyl-indole-2- carbonyl)amino]oxetan-3- yl]phenyl]-2-cyclopentyl- acetic acid | 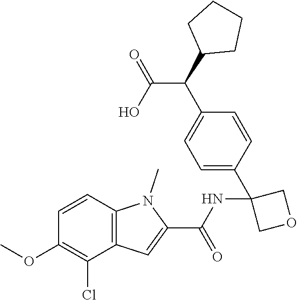 [SMILES: COc1ccc2c(cc(C(=O)NC3(c4ccc([C@@H](C(=O)O)C5CCCC5)cc4)COC3)n2C)c1Cl] |
| 7 | (±)-2-cyclobutyl-2-{4-[3-(4,5- dichloro-6-methoxy-1- methyl-1H-indole-2- amido)oxetan-3- yl]phenyl}acetic acid | 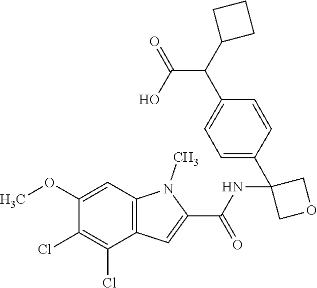 [SMILES: COc1cc2c(cc(C(=O)NC3(c4ccc(C(C(=O)O)C5CCC5)cc4)COC3)n2C)c(Cl)c1Cl] |
| 8 | (±)-2-cyclobutyl-2-{4-[3-(4,5- dichloro-1-methyl-1H-indole- 2-amido)oxetan-3- yl]phenyl}acetic acid | 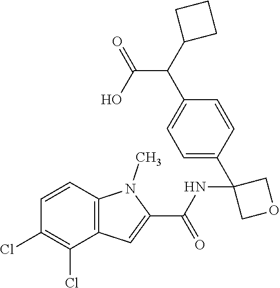 [SMILES: Cn1c(C(=O)NC2(c3ccc(C(C(=O)O)C4CCC4)cc3)COC2)cc2c(Cl)c(Cl)ccc21] |
| 9 | (±)-2-(4-(3-(4,5-Dichloro-1- methyl-1H-indole-2- carboxamido)oxetan-3- yl)phenyl)-4-methylpentanoic acid | 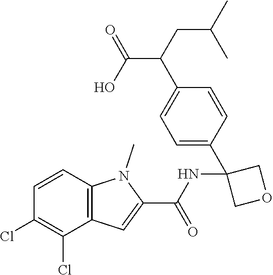 [SMILES: CC(C)CC(C(=O)O)c1ccc(C2(NC(=O)c3cc4c(Cl)c(Cl)ccc4n3C)COC2)cc1] |
| 10 | (±)-2-{4-[3-(4,5-dichloro-6- methoxy-1-methyl-1H-indole- 2-amido)oxetan-3-yl]phenyl}- 4-methylpentanoic acid | 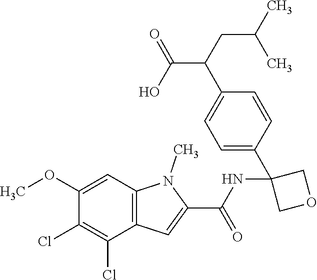 [SMILES: COc1cc2c(cc(C(=O)NC3(c4ccc(C(CC(C)C)C(=O)O)cc4)COC3)n2C)c(Cl)c1Cl] |
| 11 | 2-{4-[3-(4-chloro-5-methoxy- 1-methyl-1H-indole-2- amido)oxetan-3-yl]phenyl}-5- hydroxypentanoic acid | 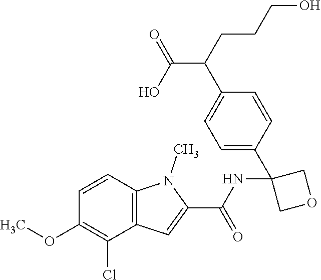 [SMILES: COc1ccc2c(cc(C(=O)NC3(c4ccc(C(CCCO)C(=O)O)cc4)COC3)n2C)c1Cl] |
| 12 | (±)-2-cyclobutyl-2-{4-[3-(4,5- dichloro-1-methyl-1H-indole- 2-amido)oxolan-3- yl]phenyl}acetic acid | 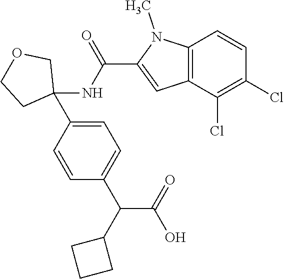 [SMILES: Cn1c(C(=O)NC2(c3ccc(C(C(=O)O)C4CCC4)cc3)CCOC2)cc2c(Cl)c(Cl)ccc21] |
| 13 | (±)-2-{4-[3-(4-chloro-5- methoxy-1-methyl-1H-indole- 2-amido)oxetan-3-yl]phenyl}- 2-cyclobutylacetic acid | 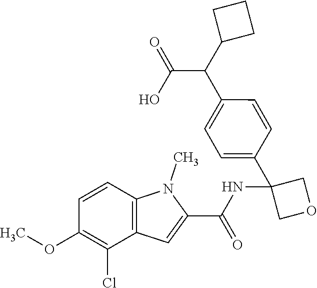 [SMILES: COc1ccc2c(cc(C(=O)NC3(c4ccc(C(C(=O)O)C5CCC5)cc4)COC3)n2C)c1Cl] |
| 14 | (±)-2-{4-[3-(4,5-dichloro-6- methoxy-1-methyl-1H-indole- 2-amido)oxetan-3- yl]phenyl}butanoic acid | 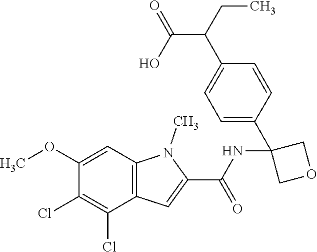 [SMILES: CCC(C(=O)O)c1ccc(C2(NC(=O)c3cc4c(Cl)c(Cl)c(OC)cc4n3C)COC2)cc1] |
| 15 | (±)-2-Cyclopropyl-2-(4-(3- (4,5-dichloro-1-methyl-1H- indole-2-carbox- amido)tetrahydro furan-3- yl)phenyl)acetic acid | 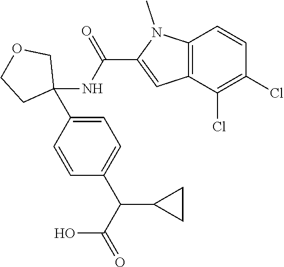 [SMILES: Cn1c(C(=O)NC2(c3ccc(C(C(=O)O)C4CC4)cc3)CCOC2)cc2c(Cl)c(Cl)ccc21] |
| 16 | (±)-2-cyclopentyl-2-{4-[3- (4,5-dichloro-1-methyl-1H- indole-2-amido)oxolan-3- yl]phenyl}acetic acid | 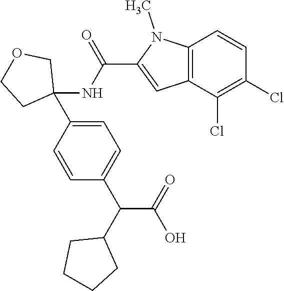 [SMILES: Cn1c(C(=O)NC2(c3ccc(C(C(=O)O)C4CCCC4)cc3)CCOC2)cc2c(Cl)c(Cl)ccc21] |
| 17 | (±)-2-{4-[3-(4,5-dichloro-6- methoxy-1-methyl-1H-indole- 2-amido)oxolan-3- yl]phenyl}pentanoic acid | 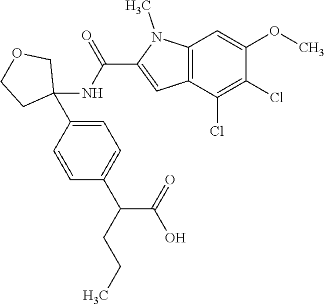 [SMILES: CCCC(C(=O)O)c1ccc(C2(NC(=O)c3cc4c(Cl)c(Cl)c(OC)cc4n3C)CCOC2)cc1] |
| 18 | (±)-2-{4-[3-(4,5-dichloro-1- methyl-1H-indole-2- amido)oxolan-3- yl]phenyl}pentanoic acid | 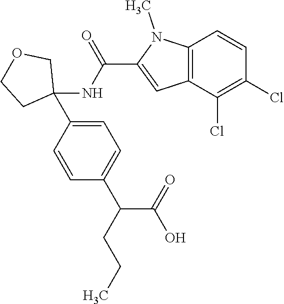 [SMILES: CCCC(C(=O)O)c1ccc(C2(NC(=O)c3cc4c(Cl)c(Cl)ccc4n3C)CCOC2)cc1] |
| 19 | (±)-2-(4-(3-(4,5-Dichloro-1- methyl-1H-indole-2- carboxamido)tetrahydro- furan-3-yl) phenyl)-3- methylbutanoic acid | 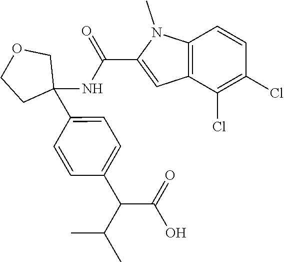 [SMILES: CC(C)C(C(=O)O)c1ccc(C2(NC(=O)c3cc4c(Cl)c(Cl)ccc4n3C)CCOC2)cc1] |
| 19 b | Diasteroisomer 1 of (±)-2-(4- (3-(4,5-Dichloro-1-methyl- 1H-indole-2- carboxamido)tetrahydro- furan-3-yl) phenyl)-3- methylbutanoic acid | 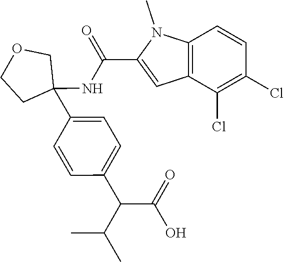 [SMILES: CC(C)C(C(=O)O)c1ccc(C2(NC(=O)c3cc4c(Cl)c(Cl)ccc4n3C)CCOC2)cc1] |
| 19 a | Diasteroisomer 2 of (±)-2-(4- (3-(4,5-Dichloro-1-methyl- 1H-indole-2- carboxamido)tetrahydro- furan-3-yl) phenyl)-3- methylbutanoic acid | 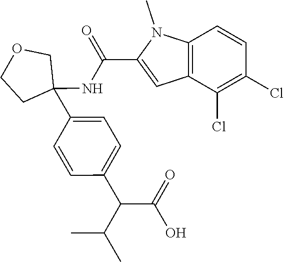 [SMILES: CC(C)C(C(=O)O)c1ccc(C2(NC(=O)c3cc4c(Cl)c(Cl)ccc4n3C)CCOC2)cc1] |
| 19 c | Diasteroisomer 3 of (±)-2-(4- (3-(4,5-Dichloro-1-methyl- 1H-indole-2- carboxamido)tetrahydro- furan-3-yl) phenyl)-3- methylbutanoic acid | 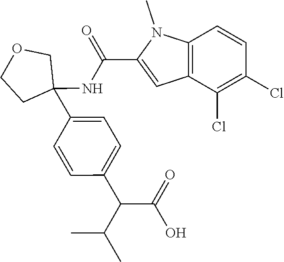 [SMILES: CC(C)C(C(=O)O)c1ccc(C2(NC(=O)c3cc4c(Cl)c(Cl)ccc4n3C)CCOC2)cc1] |
| 19 d | Diasteroisomer 4 of (±)-2-(4- (3-(4,5-Dichloro-1-methyl- 1H-indole-2- carboxamido)tetrahydro- furan-3-yl) phenyl)-3- methylbutanoic acid | 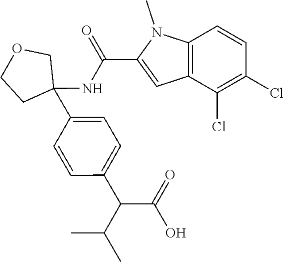 [SMILES: CC(C)C(C(=O)O)c1ccc(C2(NC(=O)c3cc4c(Cl)c(Cl)ccc4n3C)CCOC2)cc1] |
| 20 | (±)-2-{4-[3-(4,5-dichloro-6- methoxy-1-methyl-1H-indole- 2-amido)oxolan-3- yl]phenyl}-3-methylbutanoic acid | 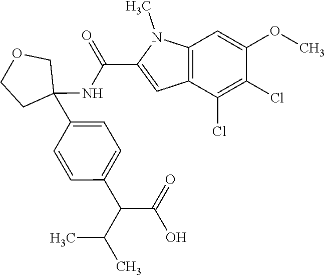 [SMILES: COc1cc2c(cc(C(=O)NC3(c4ccc(C(C(=O)O)C(C)C)cc4)CCOC3)n2C)c(Cl)c1Cl] |
| 21 | (±)-2-[4-[3-[(4-Chloro-5- methoxy-1-methyl-indole-2- carbonyl)amino]-oxetan-3- yl]phenyl]-3-methyl-butanoic acid | 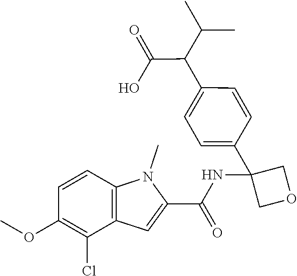 [SMILES: COc1ccc2c(cc(C(=O)NC3(c4ccc(C(C(=O)O)C(C)C)cc4)COC3)n2C)c1Cl] |
| 22 | (±)-2-cyclopentyl-2-{4-[3- (4,5-dichloro-6-methoxy-1- methyl-1H-indole-2- amido)oxetan-3- yl]phenyl}acetic acid | 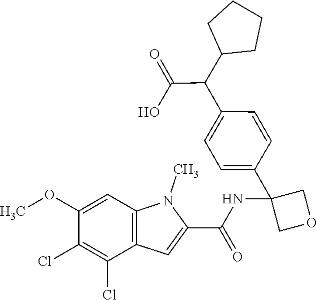 [SMILES: COc1cc2c(cc(C(=O)NC3(c4ccc(C(C(=O)O)C5CCCC5)cc4)COC3)n2C)c(Cl)c1Cl] |
| 23 | (±)-2-{4-[3-(4,5-dichloro-6- methoxy-1-methyl-1H-indole- 2-amido)oxolan-3- yl]phenyl}butanoic acid | 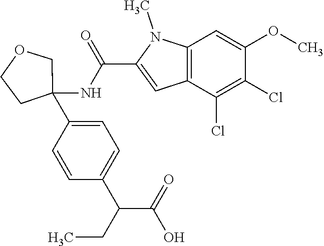 [SMILES: CCC(C(=O)O)c1ccc(C2(NC(=O)c3cc4c(Cl)c(Cl)c(OC)cc4n3C)CCOC2)cc1] |
| 24 | (±)-2-{4-[3-(4,5-dichloro-1- methyl-1H-indole-2- amido)oxetan-3- yl]phenyl}pentanoic acid | 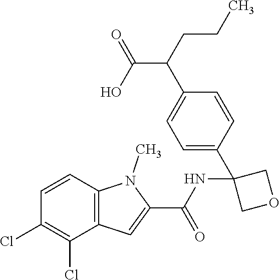 [SMILES: CCCC(C(=O)O)c1ccc(C2(NC(=O)c3cc4c(Cl)c(Cl)ccc4n3C)COC2)cc1] |
| 25 | (±)-2-Cyclopentyl-2-[4-[3- [(4,5-dichloro-1-methyl- indole-2-carbonyl) amino]oxetan-3- yl]phenyl]acetic acid | 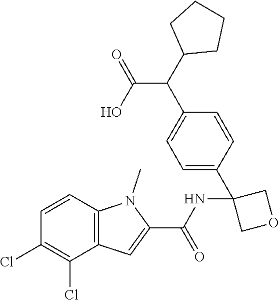 [SMILES: Cn1c(C(=O)NC2(c3ccc(C(C(=O)O)C4CCCC4)cc3)COC2)cc2c(Cl)c(Cl)ccc21] |
| 26 | (±)-2-[4-[3-[(4-chloro-5- methoxy-1-methyl-indole-2- carbonyl)amino]-oxetan-3- yl]phenyl]-2-cyclopentyl- acetic acid | 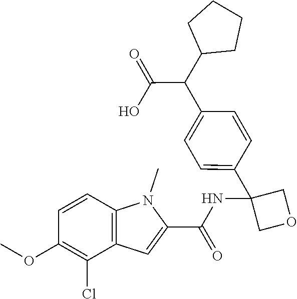 [SMILES: COc1ccc2c(cc(C(=O)NC3(c4ccc(C(C(=O)O)C5CCCC5)cc4)COC3)n2C)c1Cl] |
| 27 | 2-{4-[3-(4-chloro-5-methoxy- 1-methyl-1H-indole-2- amido)oxetan-3-yl]phenyl}-2- methylpropanoic acid | 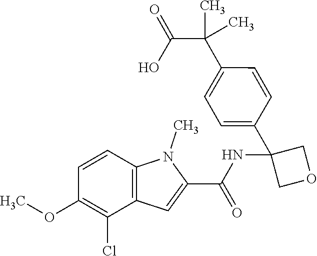 [SMILES: COc1ccc2c(cc(C(=O)NC3(c4ccc(C(C)(C)C(=O)O)cc4)COC3)n2C)c1Cl] |
| 28 | 1-{4-[3-(4,5-dichloro-1- methyl-1H-indole-2- amido)oxetan-3- yl]phenyl}cyclopropane-1- carboxylic acid | 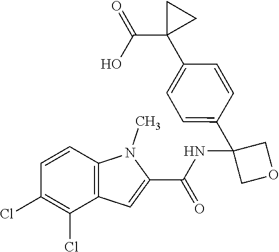 [SMILES: Cn1c(C(=O)NC2(c3ccc(C4(C(=O)O)CC4)cc3)COC2)cc2c(Cl)c(Cl)ccc21] |
| 29 | (±)-2-[4-[3-[(4,5-Dichloro-6- methoxy-1-methyl-indole-2- carbonyl)amino]oxetan-3-yl] phenyl]-3-methyl-butanoic acid | 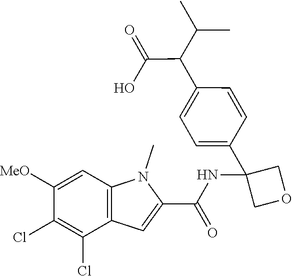 [SMILES: COc1cc2c(cc(C(=O)NC3(c4ccc(C(C(=O)O)C(C)C)cc4)COC3)n2C)c(Cl)c1Cl] |
| 30 | (±)-2-[4-[3-[(4,5-Dichloro-1- methyl-indole-2- carbonyl)amino]oxetan-3- yl]phenyl]-3-methyl-butanoic acid | 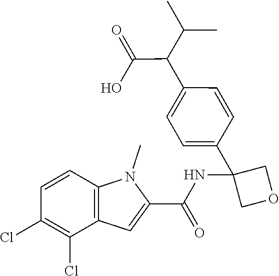 [SMILES: CC(C)C(C(=O)O)c1ccc(C2(NC(=O)c3cc4c(Cl)c(Cl)ccc4n3C)COC2)cc1] |
| 31 | (±)-2-{4-[3-(4-chloro-5- methoxy-1-methyl-1H-indole- 2-amido)oxetan-3- yl]phenyl}pentanoic acid | 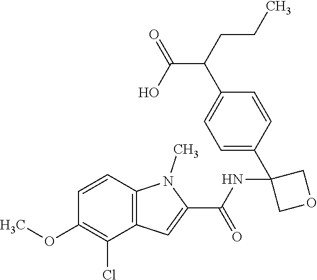 [SMILES: CCCC(C(=O)O)c1ccc(C2(NC(=O)c3cc4c(Cl)c(OC)ccc4n3C)COC2)cc1] |
| 32 | 2-{3-chloro-4-[3-(4-chloro-5- methoxy-1-methyl-1H-indole- 2-amido)oxetan-3- yl]phenyl}acetic acid | 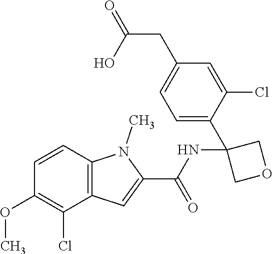 [SMILES: COc1ccc2c(cc(C(=O)NC3(c4ccc(CC(=O)O)cc4Cl)COC3)n2C)c1Cl] |
| 33 | 2-{3-chloro-4-[3-(4,5- dichloro-1-methyl-1H-indole- 2-amido)oxetan-3- yl]phenyl}propanoic acid | 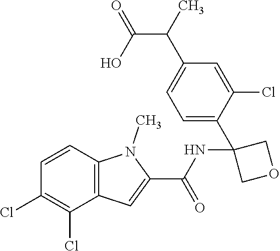 [SMILES: CC(C(=O)O)c1ccc(C2(NC(=O)c3cc4c(Cl)c(Cl)ccc4n3C)COC2)c(Cl)c1] |
| 34 | (R)-2-[4-[3-[(4,5-dichloro-1- methyl-indole-2- carbonyl)amino] oxetan-3- yl]phenyl]propanoic acid | 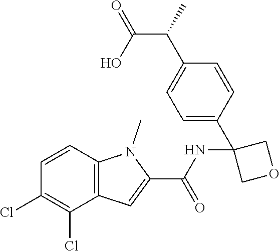 [SMILES: C[C@@H](C(=O)O)c1ccc(C2(NC(=O)c3cc4c(Cl)c(Cl)ccc4n3C)COC2)cc1] |
| 35 | (S)-2-[4-[3-[(4,5-dichloro-1- methyl-indole-2- carbonyl)amino]oxetan-3- yl]phenyl] propanoic acid | 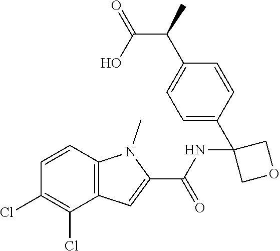 [SMILES: C[C@H](C(=O)O)c1ccc(C2(NC(=O)c3cc4c(Cl)c(Cl)ccc4n3C)COC2)cc1] |
| 36 | 2-(4-(3-(4,5-Dichloro-1- methyl-1H-indole-2- carboxamido)oxetan-3- yl)Phenyl)-2-methylpropanoic acid | 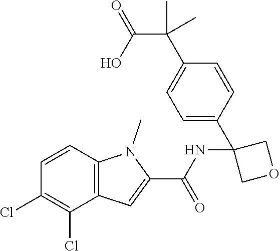 [SMILES: Cn1c(C(=O)NC2(c3ccc(C(C)(C)C(=O)O)cc3)COC2)cc2c(Cl)c(Cl)ccc21] |
| 37 | 2-{4-[3-(4,5-dichloro-1- methyl-1H-indole-2- amido)oxetan-3-yl]-3- fluorophenyl}-2- methylpropanoic acid | 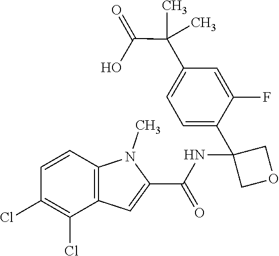 [SMILES: Cn1c(C(=O)NC2(c3ccc(C(C)(C)C(=O)O)cc3F)COC2)cc2c(Cl)c(Cl)ccc21] |
| 38 | (±)-2-{4-[3-(4,5-dichloro-6- methoxy-1-methyl-1H-indole- 2-amido)oxetan-3- yl]phenyl}pentanoic acid | 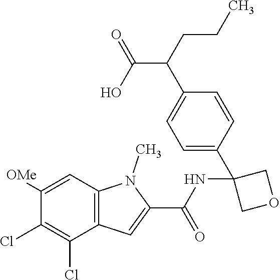 [SMILES: CCCC(C(=O)O)c1ccc(C2(NC(=O)c3cc4c(Cl)c(Cl)c(OC)cc4n3C)COC2)cc1] |
| 39 | (±)-2-{4-[3-(4-chloro-5- methoxy-1-methyl-1H-indole- 2-amido)oxolan-3-yl]-3- fluorophenyl}-2- methylpropanoic acid | 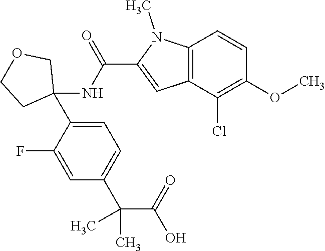 [SMILES: COc1ccc2c(cc(C(=O)NC3(c4ccc(C(C)(C)C(=O)O)cc4F)CCOC3)n2C)c1Cl] |
| 40 | (±)-2-{4-[3-(4-chloro-5- methoxy-1-methyl-1H-indole- 2-amido)oxolan-3-yl]-3- fluorophenyl}propanoic acid | 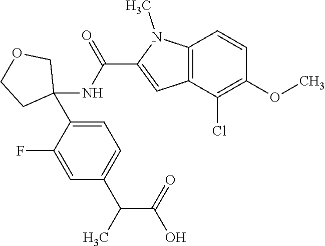 [SMILES: COc1ccc2c(cc(C(=O)NC3(c4ccc(C(C)C(=O)O)cc4F)CCOC3)n2C)c1Cl] |
| 41 | ,5-Dichloro-1-methyl-N-[3- [4-(1H-tetrazol-5- ylmethyl)phenyl]oxetan-3- yl]indole-2-carboxamide | 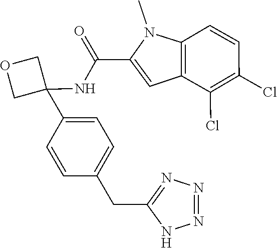 [SMILES: Cn1c(C(=O)NC2(c3ccc(Cc4nnn[nH]4)cc3)COC2)cc2c(Cl)c(Cl)ccc21] |
| 42 | (±)-2-(4-(3-(4,5-dichloro-1- methyl-1H-indole-2- carboxamido)oxetan-3- yl)phenyl)butanoic acid | 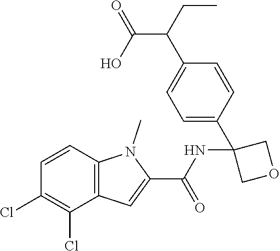 [SMILES: CCC(C(=O)O)c1ccc(C2(NC(=O)c3cc4c(Cl)c(Cl)ccc4n3C)COC2)cc1] |
| 43 | (±)-2-(4-(3-(4-chloro-5- methoxy-1-methyl-1H-indole- 2-carboxamido)oxetan-3-yl) phenyl)butanoic acid | 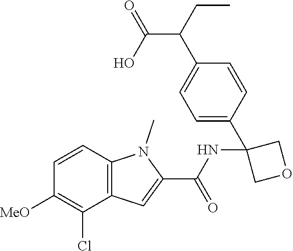 [SMILES: CCC(C(=O)O)c1ccc(C2(NC(=O)c3cc4c(Cl)c(OC)ccc4n3C)COC2)cc1] |
| 44 | 3-chloro-4-[3-(4-chloro-5- fluoro-6-methoxy-1-methyl- 1H-indole-2-amido)oxetan-3- yl]benzoic acid | 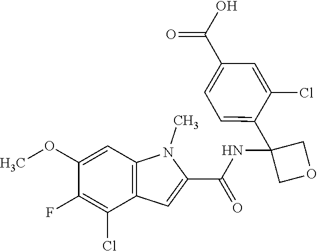 [SMILES: COc1cc2c(cc(C(=O)NC3(c4ccc(C(=O)O)cc4Cl)COC3)n2C)c(Cl)c1F] |
| 45 | 4-[3-(4-chloro-6-methoxy- 1,5-dimethyl-1H-indole-2- amido)oxetan-3-yl]-3- methylbenzoic acid | 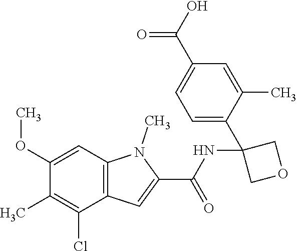 [SMILES: COc1cc2c(cc(C(=O)NC3(c4ccc(C(=O)O)cc4C)COC3)n2C)c(Cl)c1C] |
| 46 | 3-chloro-4-[3-(4-chloro-6- methoxy-1,5-dimethyl-1H- indole-2-amido)oxetan-3- yl]benzoic acid | 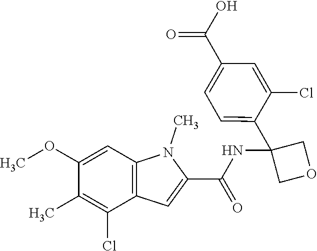 [SMILES: COc1cc2c(cc(C(=O)NC3(c4ccc(C(=O)O)cc4Cl)COC3)n2C)c(Cl)c1C] |
| 47 | 4-[3-(4-chloro-5-fluoro-6- methoxy-1-methyl-1H-indole- 2-amido)oxetan-3-yl]-3- methylbenzoic acid | 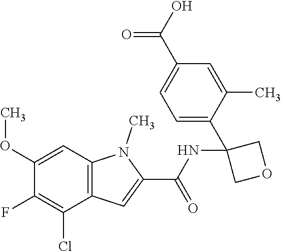 [SMILES: COc1cc2c(cc(C(=O)NC3(c4ccc(C(=O)O)cc4C)COC3)n2C)c(Cl)c1F] |
| 48 | 2-{4-[3-(4,5-dichloro-6- methoxy-1-methyl-1H-indole- 2-amido)oxetan-3- yl]phenyl}propanoic acid | 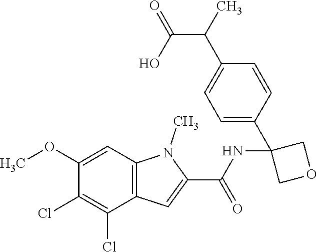 [SMILES: COc1cc2c(cc(C(=O)NC3(c4ccc(C(C)C(=O)O)cc4)COC3)n2C)c(Cl)c1Cl] |
| 49 | (±)-2-{4-[3-(4-chloro-5- methoxy-1-methyl-1H-indole- 2-amido)oxetan-3-yl]-3- fluorophenyl}propanoic acid | 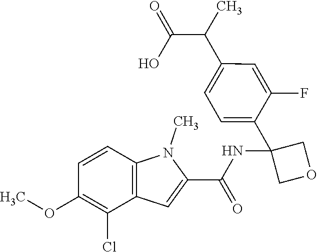 [SMILES: COc1ccc2c(cc(C(=O)NC3(c4ccc(C(C)C(=O)O)cc4F)COC3)n2C)c1Cl] |
| 50 | 4-[3-(4-chloro-6-methoxy- 1,5-dimethyl-1H-indole-2- amido)oxetan-3-yl]-3- fluorobenzoic acid | 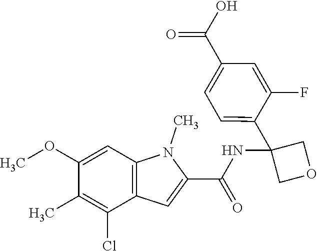 [SMILES: COc1cc2c(cc(C(=O)NC3(c4ccc(C(=O)O)cc4F)COC3)n2C)c(Cl)c1C] |
| 51 | 4-[3-(4,5-dichloro-6- methoxy-1-methyl-1H-indole- 2-amido)oxetan-3-yl]-3- methylbenzoic acid | 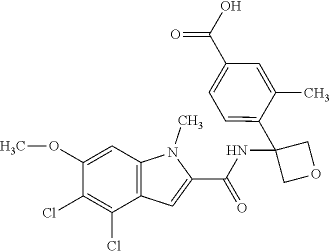 [SMILES: COc1cc2c(cc(C(=O)NC3(c4ccc(C(=O)O)cc4C)COC3)n2C)c(Cl)c1Cl] |
| 52 | (±)-N-(3-(4-(1-(1H-tetrazol-5- yl)ethyl)phenyl) tetrahydrofuran- 3-yl)-4,5-dichloro-1- methyl-1H-indole-2- carboxamide | 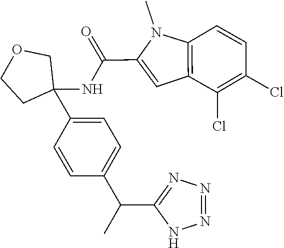 [SMILES: CC(c1ccc(C2(NC(=O)c3cc4c(Cl)c(Cl)ccc4n3C)CCOC2)cc1)c1nnn[nH]1] |
| 53 | (±)-2-{4-[3-(4,5-dichloro-6- methoxy-1-methyl-1H-indole- 2-amido)oxolan-3-yl]-3- fluorophenyl}-2- methylpropanoic acid | 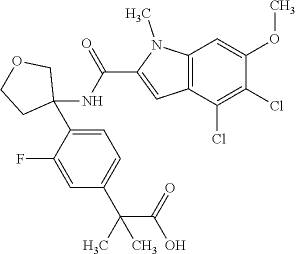 [SMILES: COc1cc2c(cc(C(=O)NC3(c4ccc(C(C)(C)C(=O)O)cc4F)CCOC3)n2C)c(Cl)c1Cl] |
| 54 | (±)-2-{4-[3-(4,5-dichloro-1- methyl-1H-indole-2- amido)oxolan-3-yl]-3- fluorophenyl}-2- methylpropanoic acid | 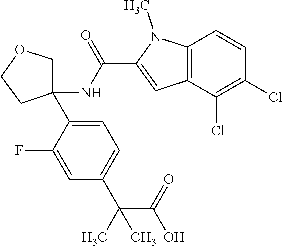 [SMILES: Cn1c(C(=O)NC2(c3ccc(C(C)(C)C(=O)O)cc3F)CCOC2)cc2c(Cl)c(Cl)ccc21] |
| 55 | (±)-2-{4-[3-(4,5-dichloro-6- methoxy-1-methyl-1H-indole- 2-amido)oxolan-3-yl]-3- fluorophenyl}propanoic acid | 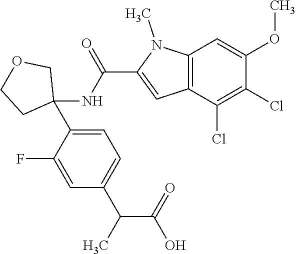 [SMILES: COc1cc2c(cc(C(=O)NC3(c4ccc(C(C)C(=O)O)cc4F)CCOC3)n2C)c(Cl)c1Cl] |
| 56 | 4-[3-(4-chloro-5-fluoro-6- methoxy-1-methyl-1H-indole- 2-amido)oxetan-3-yl]-3- fluorobenzoic acid | 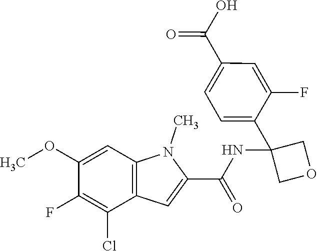 [SMILES: COc1cc2c(cc(C(=O)NC3(c4ccc(C(=O)O)cc4F)COC3)n2C)c(Cl)c1F] |
| 57 | (±)-2-{4-[3-(4,5-dichloro-1- methyl-1H-indole-2- amido)oxolan-3-yl]-3- fluorophenyl}propanoic acid | 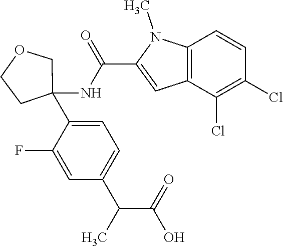 [SMILES: CC(C(=O)O)c1ccc(C2(NC(=O)c3cc4c(Cl)c(Cl)ccc4n3C)CCOC2)c(F)c1] |
| 58 | (±)-1-{4-[3-(4-chloro-5- methoxy-1-methyl-1H-indole- 2-amido)oxolan-3- yl]phenyl}cyclopropane-1- carboxylic acid | 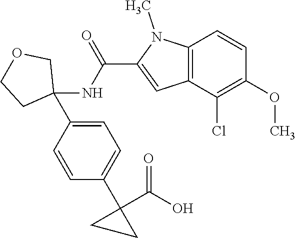 [SMILES: COc1ccc2c(cc(C(=O)NC3(c4ccc(C5(C(=O)O)CC5)cc4)CCOC3)n2C)c1Cl] |
| 59 | (±)-1-{4-[3-(4,5-dichloro-1- methyl-1H-indole-2- amido)oxolan-3- yl]phenyl}cyclopropane-1- carboxylic acid | 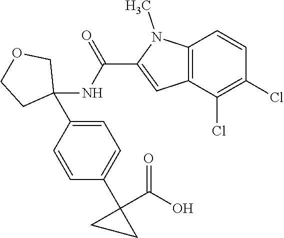 [SMILES: Cn1c(C(=O)NC2(c3ccc(C4(C(=O)O)CC4)cc3)CCOC2)cc2c(Cl)c(Cl)ccc21] |
| 60 | 4-[3-(4-chloro-5-methoxy-1- methyl-1H-indole-2- amido)oxetan-3-yl]-3- methylbenzoic acid | 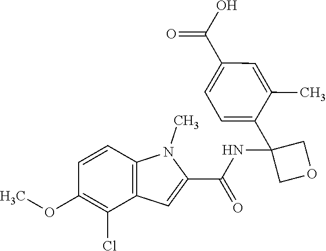 [SMILES: COc1ccc2c(cc(C(=O)NC3(c4ccc(C(=O)O)cc4C)COC3)n2C)c1Cl] |
| 61 | 2-{4-[3-(4-chloro-5-fluoro-6- methoxy-1-methyl-1H-indole- 2-amido)oxetan-3-yl]-3- fluorophenyl}acetic acid | 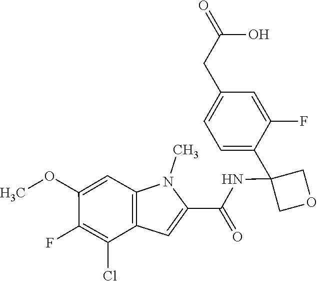 [SMILES: COc1cc2c(cc(C(=O)NC3(c4ccc(CC(=O)O)cc4F)COC3)n2C)c(Cl)c1F] |
| 62 | 2-{4-[3-(4-chloro-6-methoxy- 1,5-dimethyl-1H-indole-2- amido)oxetan-3-yl]-3- fluorophenyl}acetic acid | 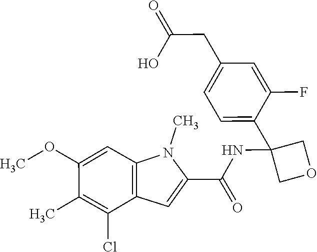 [SMILES: COc1cc2c(cc(C(=O)NC3(c4ccc(CC(=O)O)cc4F)COC3)n2C)c(Cl)c1C] |
| 63 | 2-{4-[3-(4-chloro-1,5- dimethyl-1H-indole-2- amido)oxetan-3-yl]-3- fluorophenyl}acetic acid | 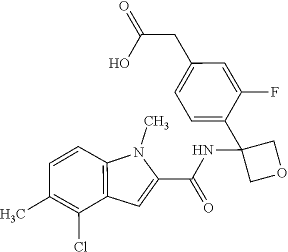 [SMILES: Cc1ccc2c(cc(C(=O)NC3(c4ccc(CC(=O)O)cc4F)COC3)n2C)c1Cl] |
| 64 | (±)-2-{4-[3-(4-chloro-5- methoxy-1-methyl-1H-indole- 2-amido)oxolan-3- yl]phenyl}propanoic acid | 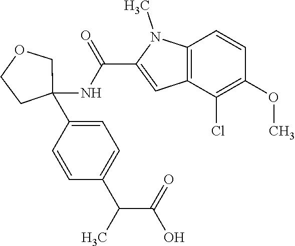 [SMILES: COc1ccc2c(cc(C(=O)NC3(c4ccc(C(C)C(=O)O)cc4)CCOC3)n2C)c1Cl] |
| 65 | 3-chloro-4-[3-(4-chloro-5- methoxy-1-methyl-1H-indole- 2-amido)oxetan-3-yl]benzoic acid | 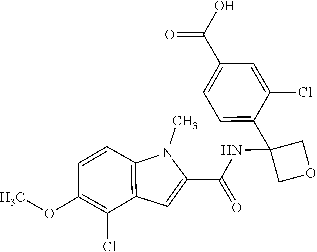 [SMILES: COc1ccc2c(cc(C(=O)NC3(c4ccc(C(=O)O)cc4Cl)COC3)n2C)c1Cl] |
| 66 | 3-chloro-4-[3-(4,5-dichloro-6- methoxy-1-methyl-1H-indole- 2-amido)oxetan-3-yl]benzoic acid | 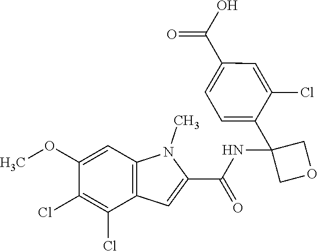 [SMILES: COc1cc2c(cc(C(=O)NC3(c4ccc(C(=O)O)cc4Cl)COC3)n2C)c(Cl)c1Cl] |
| 67 | (±)-2-{4-[3-(4-chloro-5- methoxy-1-methyl-1H-indole- 2-amido)oxetan-3- yl]phenyl}propanoic acid | 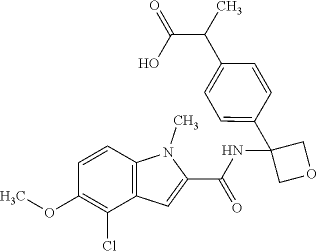 [SMILES: COc1ccc2c(cc(C(=O)NC3(c4ccc(C(C)C(=O)O)cc4)COC3)n2C)c1Cl] |
| 68 | (±)-2-{4-[3-(4,5-dichloro-1- methyl-1H-indole-2- amido)oxetan-3- yl]phenyl}propanoic acid | 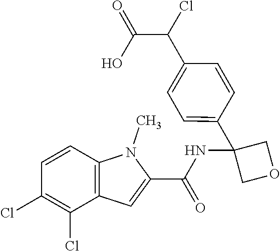 [SMILES: Cn1c(C(=O)NC2(c3ccc(C(Cl)C(=O)O)cc3)COC2)cc2c(Cl)c(Cl)ccc21] |
| 69 | 2-{4-[3-(4,5-dichloro-1- methyl-1H-indole-2- amido)oxetan-3-yl]-3- methoxyphenyl}acetic acid | 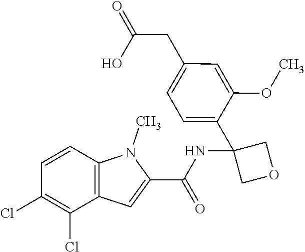 [SMILES: COc1cc(CC(=O)O)ccc1C1(NC(=O)c2cc3c(Cl)c(Cl)ccc3n2C)COC1] |
| 70 | 2-{4-[3-(4,5-dichloro-1- methyl-1H-indole-2- amido)oxetan-3-yl]-3- fluorophenyl}propanoic acid | 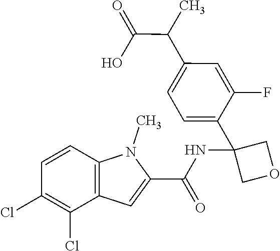 [SMILES: CC(C(=O)O)c1ccc(C2(NC(=O)c3cc4c(Cl)c(Cl)ccc4n3C)COC2)c(F)c1] |
| 71 | (±)-2-[3-Cyano-4-[3-[(4,5- dichloro-1-methyl-indole-2- carbonyl)amino]tetrahydro- furan-3-yl]phenyl]acetic acid | 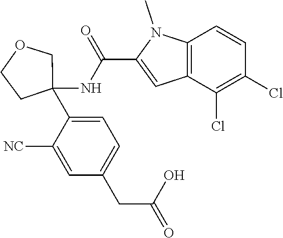 [SMILES: Cn1c(C(=O)NC2(c3ccc(CC(=O)O)cc3C#N)CCOC2)cc2c(Cl)c(Cl)ccc21] |
| 72 | (±)-2-{4-[3-(4,5-dichloro-1- methyl-1H-indole-2- amido)oxolan-3- yl]phenyl}butanoic acid | 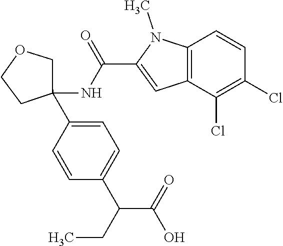 [SMILES: CCC(C(=O)O)c1ccc(C2(NC(=O)c3cc4c(Cl)c(Cl)ccc4n3C)CCOC2)cc1] |
| 73 | (±)-5-[3-(4,5-dichloro-1- methyl-1H-indole-2- amido)oxolan-3-yl]pyridine- 2-carboxylic acid | 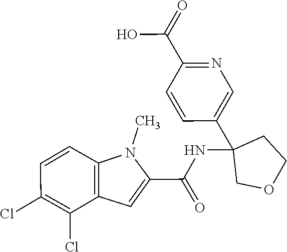 [SMILES: Cn1c(C(=O)NC2(c3ccc(C(=O)O)nc3)CCOC2)cc2c(Cl)c(Cl)ccc21] |
| 74 | (±)-6-[3-(4,5-dichloro-1- methyl-1H-indole-2- amido)oxolan-3-yl]pyridine- 3-carboxylic acid | 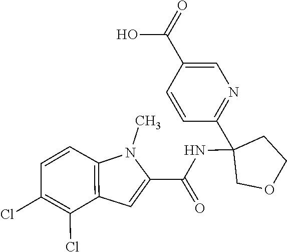 [SMILES: Cn1c(C(=O)NC2(c3ccc(C(=O)O)cn3)CCOC2)cc2c(Cl)c(Cl)ccc21] |
| 75 | 4-[3-(4-chloro-5-methoxy-1- methyl-1H-indole-2- amido)oxetan-3-yl]-3- fluorobenzoic acid | 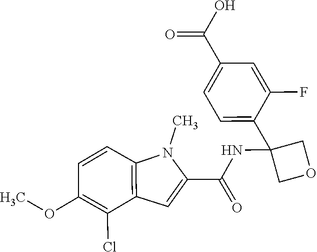 [SMILES: COc1ccc2c(cc(C(=O)NC3(c4ccc(C(=O)O)cc4F)COC3)n2C)c1Cl] |
| 76 | 4-[3-(4,5-dichloro-6- methoxy-1-methyl-1H-indole- 2-amido)oxetan-3-yl]-3- fluorobenzoic acid | 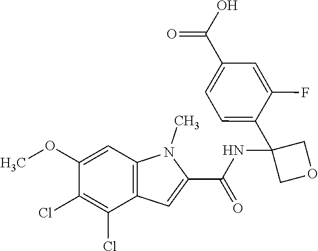 [SMILES: COc1cc2c(cc(C(=O)NC3(c4ccc(C(=O)O)cc4F)COC3)n2C)c(Cl)c1Cl] |
| 77 | (±)-2-{4-[3-(4-chloro-5- methoxy-1-methyl-1H-indole- 2-amido)oxolan-3- yl]phenyl}-2- methylpropanoic acid | 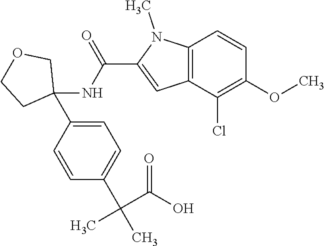 [SMILES: COc1ccc2c(cc(C(=O)NC3(c4ccc(C(C)(C)C(=O)O)cc4)CCOC3)n2C)c1Cl] |
| 78 | (±)-2-{4-[3-(4-chloro-5- methoxy-1-methyl-1H-indole- 2-amido)oxolan-3- yl]phenyl}acetic acid | 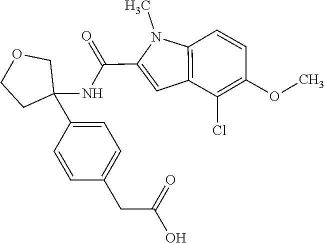 [SMILES: COc1ccc2c(cc(C(=O)NC3(c4ccc(CC(=O)O)cc4)CCOC3)n2C)c1Cl] |
| 79 | 4-[3-(4,5-dichloro-1-methyl- 1H-indole-2-amido)oxetan-3- yl]-3-fluorobenzoic acid | 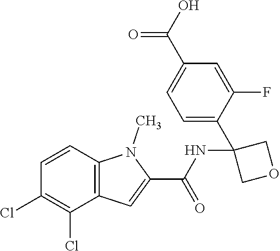 [SMILES: Cn1c(C(=O)NC2(c3ccc(C(=O)O)cc3F)COC2)cc2c(Cl)c(Cl)ccc21] |
| 80 | (±)-4-{3-[4,5-dichloro-1- methyl-6-(oxetan-3- ylmethoxy)-1H-indole-2- amido]oxolan-3-yl}benzoic acid | 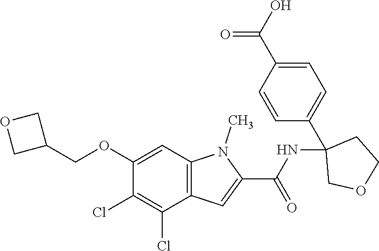 [SMILES: Cn1c(C(=O)NC2(c3ccc(C(=O)O)cc3)CCOC2)cc2c(Cl)c(Cl)c(OCC3COC3)cc21] |
| 81 | (±)-4-(3-(6-((1H-imidazol-2- yl)methoxy)-4,5-dichloro-1- methyl-1H-indole-2- carboxamido) tetrahydrofuran-3-yl)benzoic acid | 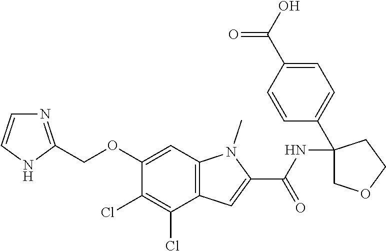 [SMILES: Cn1c(C(=O)NC2(c3ccc(C(=O)O)cc3)CCOC2)cc2c(Cl)c(Cl)c(OCc3ncc[nH]3)cc21] |
| 82 | 2-{4-[3-(4,5-dichloro-1- methyl-1H-indole-2- amido)oxetan-3-yl]-3- methylphenyl}acetic acid | 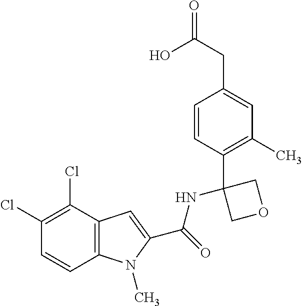 [SMILES: Cc1cc(CC(=O)O)ccc1C1(NC(=O)c2cc3c(Cl)c(Cl)ccc3n2C)COC1] |
| 83 | 4-[3-(4,5-dichloro-1-methyl- 1H-indole-2-amido)oxetan-3- yl]-3-methylbenzoic acid | 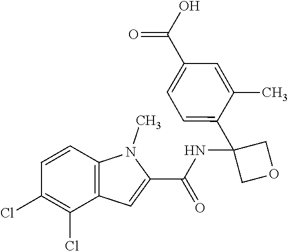 [SMILES: Cc1cc(C(=O)O)ccc1C1(NC(=O)c2cc3c(Cl)c(Cl)ccc3n2C)COC1] |
| 84 | 2-{3-chloro-4-[3-(4,5- dichloro-1-methyl-1H-indole- 2-amido)oxetan-3- yl]phenyl}acetic acid | 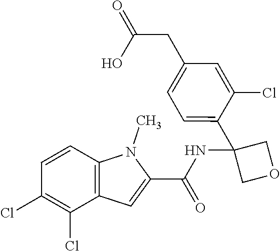 [SMILES: Cn1c(C(=O)NC2(c3ccc(CC(=O)O)cc3Cl)COC2)cc2c(Cl)c(Cl)ccc21] |
| 85 | 3-chloro-4-[3-(4,5-dichloro-1- methyl-1H-indole-2- amido)oxetan-3-yl]benzoic acid | 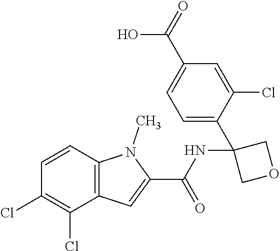 [SMILES: Cn1c(C(=O)NC2(c3ccc(C(=O)O)cc3Cl)COC2)cc2c(Cl)c(Cl)ccc21] |
| 86 | 2-{4-[3-(4,5-dichloro-6- methoxy-1-methyl-1H-indole- 2-amido)oxetan-3-yl]-3- fluorophenyl}acetic acid | 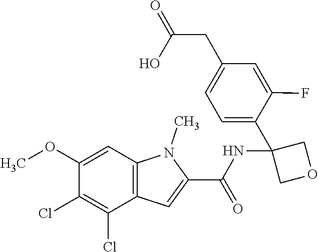 [SMILES: COc1cc2c(cc(C(=O)NC3(c4ccc(CC(=O)O)cc4F)COC3)n2C)c(Cl)c1Cl] |
| 87 | (±)-2-{4-[3-(4,5-dichloro-1- methyl-1H-indole-2- amido)oxolan-3-yl]phenyl}- 2-methylpropanoic acid | 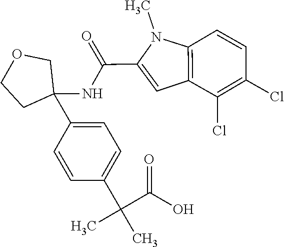 [SMILES: Cn1c(C(=O)NC2(c3ccc(C(C)(C)C(=O)O)cc3)CCOC2)cc2c(Cl)c(Cl)ccc21] |
| 88 | (±)-4-[3-[(4,5-Dichloro-1- methyl-indole-2- carbonyl)amino]tetrahydro- furan-3-yl]-2-ethyl-benzoic acid | 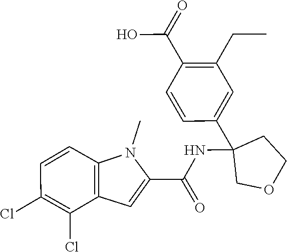 [SMILES: CCc1cc(C2(NC(=O)c3cc4c(Cl)c(Cl)ccc4n3C)CCOC2)ccc1C(=O)O] |
| 89 | (±)-4-(3-(6-(3-Amino-2- hydroxypropoxy)-4,5- dichloro-1-methyl-1H-indole- 2-carboxamido) tetrahydrofuran-3-yl)benzoic acid | 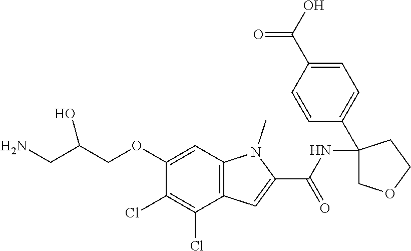 [SMILES: Cn1c(C(=O)NC2(c3ccc(C(=O)O)cc3)CCOC2)cc2c(Cl)c(Cl)c(OCC(O)CN)cc21] |
| 90 | 2-{4-[3-(4,5-dichloro-1- methyl-1H-indole-2- amido)oxetan-3-yl]-3- fluorophenyl}acetic acid | 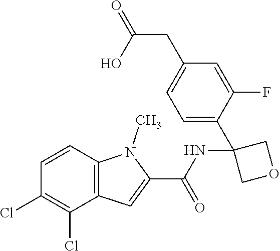 [SMILES: Cn1c(C(=O)NC2(c3ccc(CC(=O)O)cc3F)COC2)cc2c(Cl)c(Cl)ccc21] |
| 91 | (±)-4,5-Dichloro-N-(3-(4- (cyanomethyl)phenyl)tetrahydro- furan-3-yl)-1-methyl-1H- indole-2-carboxamide | 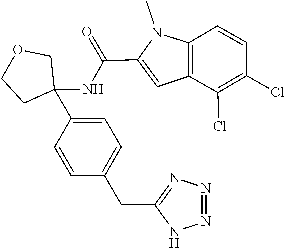 [SMILES: Cn1c(C(=O)NC2(c3ccc(Cc4nnn[nH]4)cc3)CCOC2)cc2c(Cl)c(Cl)ccc21] |
| 92 | (±)-4-{3-[4,5-dichloro-1- methyl-6-(oxolan-3- ylmethoxy)-1H-indole-2- amido]oxolan-3-yl}benzoic acid | 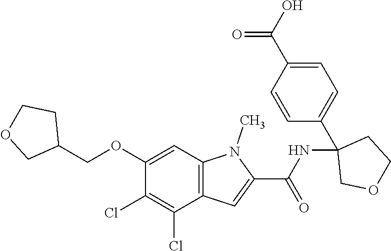 [SMILES: Cn1c(C(=O)NC2(c3ccc(C(=O)O)cc3)CCOC2)cc2c(Cl)c(Cl)c(OCC3CCOC3)cc21] |
| 93 | (±)-4-[3-[[4,5-Dichloro-1- methyl-6-[(3-methyl-2-oxo- oxazolidin-5-yl) methoxy]indole-2- carbonyl]amino] tetrahydrofuran- 3-yl]benzoic acid | 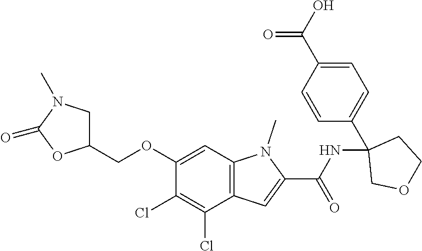 [SMILES: CN1CC(COc2cc3c(cc(C(=O)NC4(c5ccc(C(=O)O)cc5)CCOC4)n3C)c(Cl)c2Cl)OC1=O] |
| 94 | (±)-4-{3-[4,5-dichloro-1- methyl-6-(oxolan-2- ylmethoxy)-1H-indole-2- amido]oxolan-3-yl}benzoic acid | 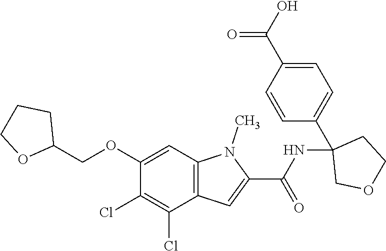 [SMILES: Cn1c(C(=O)NC2(c3ccc(C(=O)O)cc3)CCOC2)cc2c(Cl)c(Cl)c(OCC3CCCO3)cc21] |
| 95 | (±)-4-(3-{4-chloro-1-methyl- 6-[(2-oxo-1,3-oxazolidin-5- yl)methoxy]-1H-indole-2- amido}oxolan-3-yl)benzoic acid | 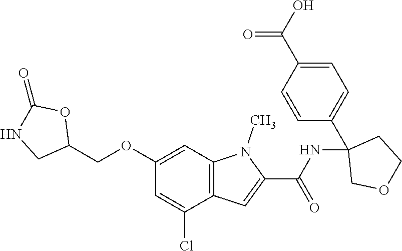 [SMILES: Cn1c(C(=O)NC2(c3ccc(C(=O)O)cc3)CCOC2)cc2c(Cl)cc(OCC3CNC(=O)O3)cc21] |
| 96 | (±)-4-[3-(4-chloro-5- cyclopropyl-1-methyl-1H- indole-2-amido)oxolan-3- yl]benzoic acid | 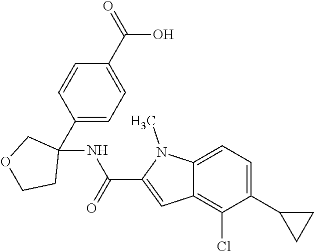 [SMILES: Cn1c(C(=O)NC2(c3ccc(C(=O)O)cc3)CCOC2)cc2c(Cl)c(C3CC3)ccc21] |
| 97 | (±)-4-(3-{4,5-dichloro-6-[2- (dimethylamino)ethoxy]-1- methyl-1H-indole-2- amido}oxolan-3-yl)benzoic acid | 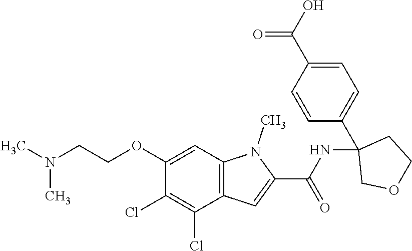 [SMILES: CN(C)CCOc1cc2c(cc(C(=O)NC3(c4ccc(C(=O)O)cc4)CCOC3)n2C)c(Cl)c1Cl] |
| 98 | (±)-4-[3-[(4,5-Dichloro-1- methyl-indole-2- carbonyl)amino]tetrahydro- furan-3-yl]-2-methyl-benzoic acid | 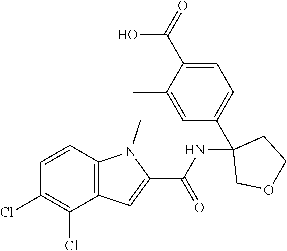 [SMILES: Cc1cc(C2(NC(=O)c3cc4c(Cl)c(Cl)ccc4n3C)CCOC2)ccc1C(=O)O] |
| 99 | (±)-4-[3-[(4,5-dichloro-1- methyl-indole-2- carbonyl)amino]tetrahydro- furan-3-yl]-3-methyl-benzoic acid | 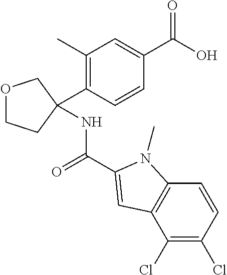 [SMILES: Cc1cc(C(=O)O)ccc1C1(NC(=O)c2cc3c(Cl)c(Cl)ccc3n2C)CCOC1] |
| 100 | (±)-4-{3-[4,5-dichloro-6-(2- methoxyethoxy)-1-methyl- 1H-indole-2-amido]oxolan-3- yl}benzoic acid | 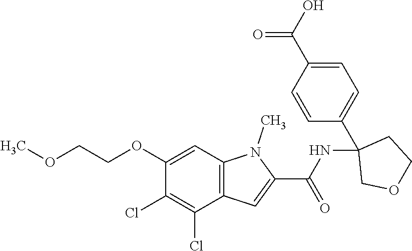 [SMILES: COCCOc1cc2c(cc(C(=O)NC3(c4ccc(C(=O)O)cc4)CCOC3)n2C)c(Cl)c1Cl] |
| 101 | (±)-2-cyclopropyl-4-[3-[(4,5- dichloro-1-methyl-indole-2- carbonyl)amino]- tetrahydrofuran-3-yl]benzoic acid | 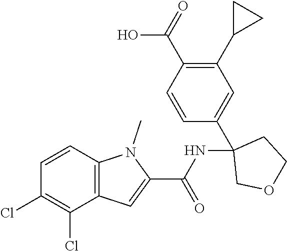 [SMILES: Cn1c(C(=O)NC2(c3ccc(C(=O)O)c(C4CC4)c3)CCOC2)cc2c(Cl)c(Cl)ccc21] |
| 102 | (±)-4-(3-{4,5-dichloro-1- methyl-6-[(1-methyl-5- oxopyrrolidin-3-yl)methoxy]- 1H-indole-2-amido}oxolan-3- yl)benzoic acid | 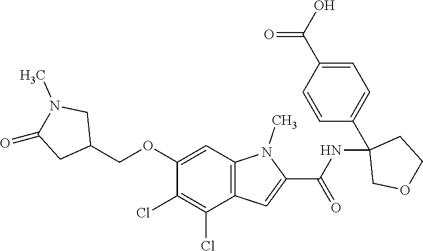 [SMILES: CN1CC(COc2cc3c(cc(C(=O)NC4(c5ccc(C(=O)O)cc5)CCOC4)n3C)c(Cl)c2Cl)CC1=O] |
| 103 | 2-{4-[3-(4-chloro-5-methoxy- 1-methyl-1H-indole-2- amido)oxetan-3- yl]phenyl}acetic acid | 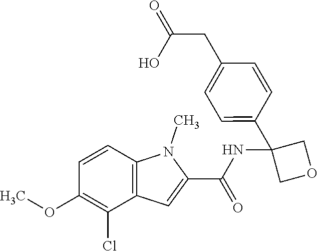 [SMILES: COc1ccc2c(cc(C(=O)NC3(c4ccc(CC(=O)O)cc4)COC3)n2C)c1Cl] |
| 104 | (±)-4-[3-(4,5-dichloro-6- ethoxy-1-methyl-1H-indole- 2-amido)oxolan-3-yl]benzoic acid | 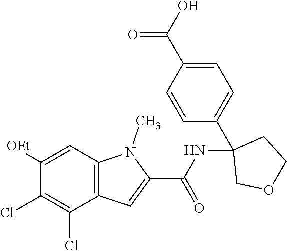 [SMILES: CCOc1cc2c(cc(C(=O)NC3(c4ccc(C(=O)O)cc4)CCOC3)n2C)c(Cl)c1Cl] |
| 105 | (±)-2-[4-[3-[(4,5-dichloro-1- methyl-indole-2- carbonyl)amino]tetrahydrofuran- 3-yl]phenyl] propanoic acid | 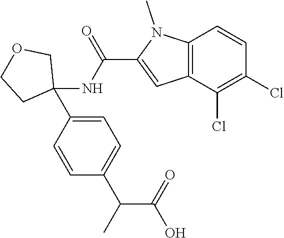 [SMILES: CC(C(=O)O)c1ccc(C2(NC(=O)c3cc4c(Cl)c(Cl)ccc4n3C)CCOC2)cc1] |
| 106 | (±)-4-[3-(4-chloro-5-fluoro-1- methyl-1H-indole-2- amido)oxolan-3-yl]benzoic acid | 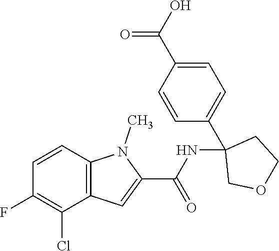 [SMILES: Cn1c(C(=O)NC2(c3ccc(C(=O)O)cc3)CCOC2)cc2c(Cl)c(F)ccc21] |
| 107 | (±)-3-chloro-4-[3-(4,5- dichloro-1-methyl-1H-indole- 2-amido)oxolan-3-yl]benzoic acid | 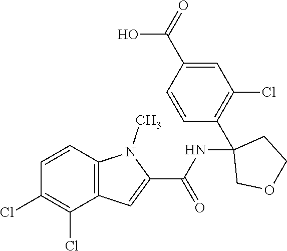 [SMILES: Cn1c(C(=O)NC2(c3ccc(C(=O)O)cc3Cl)CCOC2)cc2c(Cl)c(Cl)ccc21] |
| 108 | (R)-2-(4-(3-(4,5-dichloro-1- methyl-1H-indole-2- carboxamido) tetrahydrofuran-3- yl)phenyl)acetic acid | 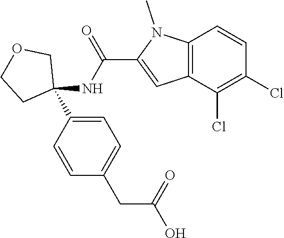 [SMILES: Cn1c(C(=O)N[C@@]2(c3ccc(CC(=O)O)cc3)CCOC2)cc2c(Cl)c(Cl)ccc21] |
| 109 | (S)-2-(4-(3-(4,5-dichloro-1- methyl-1H-indole-2- carboxamido)- tetrahydrofuran-3-yl) phenyl)acetic acid | 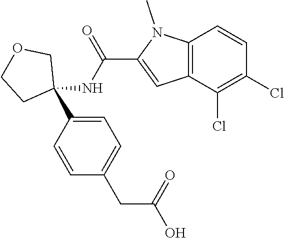 [SMILES: Cn1c(C(=O)N[C@]2(c3ccc(CC(=O)O)cc3)CCOC2)cc2c(Cl)c(Cl)ccc21] |
| 110 | (±)-4-[1-Acetyl-3-[(4,5- dichloro-1-methyl-indole-2- carbonyl)amino]pyrrolidin-3- yl]benzoic acid | 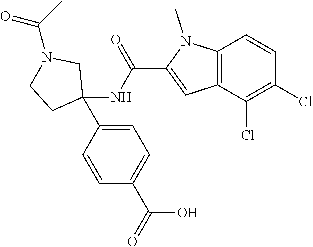 [SMILES: CC(=O)N1CCC(NC(=O)c2cc3c(Cl)c(Cl)ccc3n2C)(c2ccc(C(=O)O)cc2)C1] |
| 111 | (±)-4-[3-[[4,5-Dichloro-1- methyl-6-[(2-oxooxazolidin- 5-yl)methoxy]indole-2- carbonyl] amino]tetrahydrofuran-3- yl]benzoic acid | 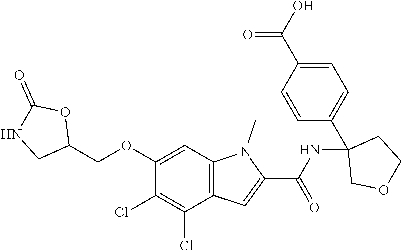 [SMILES: Cn1c(C(=O)NC2(c3ccc(C(=O)O)cc3)CCOC2)cc2c(Cl)c(Cl)c(OCC3CNC(=O)O3)cc21] |
| 112 | 2-(4-(3-(4,5-Dichloro-1- methyl-1H-indole-2- carboxamido)oxetan-3-yl)- phenyl)acetic acid | 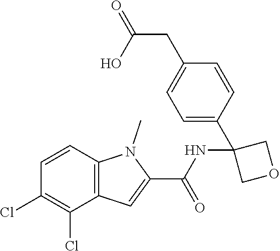 [SMILES: Cn1c(C(=O)NC2(c3ccc(CC(=O)O)cc3)COC2)cc2c(Cl)c(Cl)ccc21] |
| 113 | (±)-4-(3-{9-chloro-6-methyl- 2H,3H,6H-[1,4]dioxino[2,3- f]indole-7-amido}oxolan-3- yl)benzoic acid | 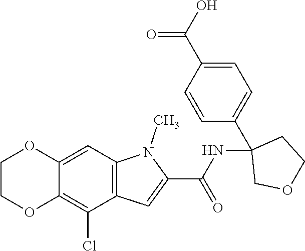 [SMILES: Cn1c(C(=O)NC2(c3ccc(C(=O)O)cc3)CCOC2)cc2c(Cl)c3c(cc21)OCCO3] |
| 114 | (±)-4-[3-(4-chloro-6- methoxy-1-methyl-1H-indole- 2-amido)oxolan-3-yl]benzoic acid | 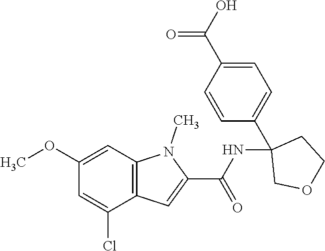 [SMILES: COc1cc(Cl)c2cc(C(=O)NC3(c4ccc(C(=O)O)cc4)CCOC3)n(C)c2c1] |
| 115 | (±)-4-[3-(4,5-dichloro-6- methoxy-1-methyl-1H-indole- 2-amido)oxolan-3-yl]benzoic acid | 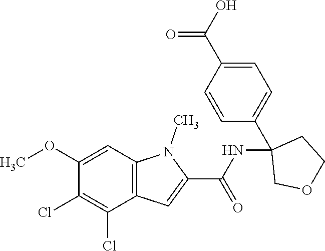 [SMILES: COc1cc2c(cc(C(=O)NC3(c4ccc(C(=O)O)cc4)CCOC3)n2C)c(Cl)c1Cl] |
| 116 | (±)-4-[3-(4-chloro-1,5- dimethyl-1H-indole-2- amido)oxolan-3-yl]benzoic acid | 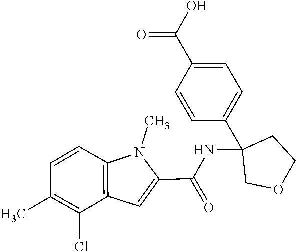 [SMILES: Cc1ccc2c(cc(C(=O)NC3(c4ccc(C(=O)O)cc4)CCOC3)n2C)c1Cl] |
| 117 | (±)-4-[3-(4-chloro-1-methyl- 1H-indole-2-amido)oxolan-3- yl]benzoic acid | 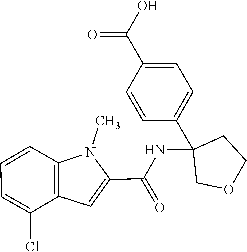 [SMILES: Cn1c(C(=O)NC2(c3ccc(C(=O)O)cc3)CCOC2)cc2c(Cl)cccc21] |
| 118 | (±)-4-[3-(4,5-dichloro-1- methyl-1H-indole-2- amido)oxolan-3-yl]-3- fluorobenzoic acid | 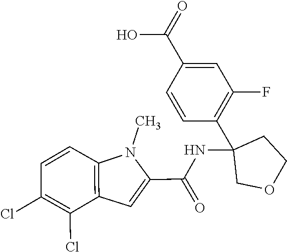 [SMILES: Cn1c(C(=O)NC2(c3ccc(C(=O)O)cc3F)CCOC2)cc2c(Cl)c(Cl)ccc21] |
| 119 | (±)-2-[3-[3-[(4,5-Dichloro-1- methyl-indole-2- carbonyl)amino] tetrahydrofuran- 3-yl]phenyl] acetic acid | 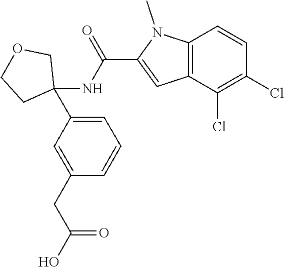 [SMILES: Cn1c(C(=O)NC2(c3cccc(CC(=O)O)c3)CCOC2)cc2c(Cl)c(Cl)ccc21] |
| 120 | (±)-2-Cyano-4-(3-(4,5- dichloro-1-methyl-1H-indole- 2-carboxamido) tetrahydrofuran-3-yl)benzoic acid | 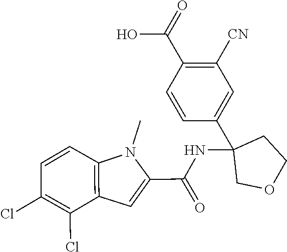 [SMILES: Cn1c(C(=O)NC2(c3ccc(C(=O)O)c(C#N)c3)CCOC2)cc2c(Cl)c(Cl)ccc21] |
| 121 | (±)-4-[3-[(4,5-Dichloro-1- methyl-indole-2- carbonyl)amino]-1-phenyl- pyrrolidin-3-yl]benzoic acid | 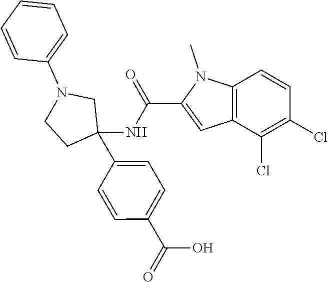 [SMILES: Cn1c(C(=O)NC2(c3ccc(C(=O)O)cc3)CCN(c3ccccc3)C2)cc2c(Cl)c(Cl)ccc21] |
| 122 | (±)-4-[3-(4-chloro-5- methoxy-1-methyl-1H-indole- 2-amido)oxolan-3-yl]benzoic acid | 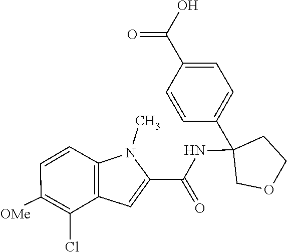 [SMILES: COc1ccc2c(cc(C(=O)NC3(c4ccc(C(=O)O)cc4)CCOC3)n2C)c1Cl] |
| 123 | (±)-4-[3-(4,5-dichloro-1- methyl-1H-indole-2- amido)oxan-3-yl]benzoic acid | 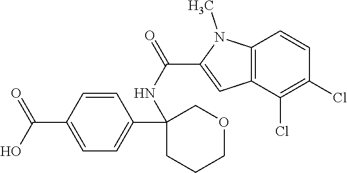 [SMILES: Cn1c(C(=O)NC2(c3ccc(C(=O)O)cc3)CCCOC2)cc2c(Cl)c(Cl)ccc21] |
| 124 | 4-[4-(4,5-dichloro-1-methyl- 1H-indole-2-amido)oxan-4- yl]benzoic acid | 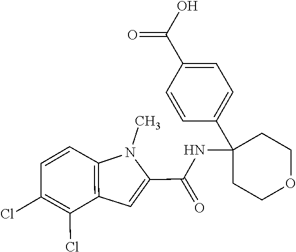 [SMILES: Cn1c(C(=O)NC2(c3ccc(C(=O)O)cc3)CCOCC2)cc2c(Cl)c(Cl)ccc21] |
| 125 | (±)-4-[3-[(4,5-Dichloro-1- methyl-indole-2- carbonyl)amino]-1-(2,2,2- trifluoroethyl)pyrrolidin-3- yl]benzoic acid | 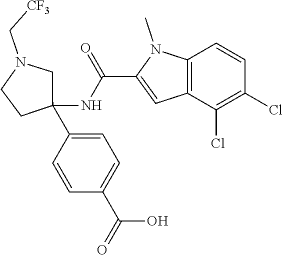 [SMILES: Cn1c(C(=O)NC2(c3ccc(C(=O)O)cc3)CCN(CC(F)(F)F)C2)cc2c(Cl)c(Cl)ccc21] |
| 126 | (±)-4-[3-[(4,5-Dichloro-1- methyl-indole-2- carbonyl)amino]-1-ethyl- pyrrolidin-3-yl]benzoic acid | 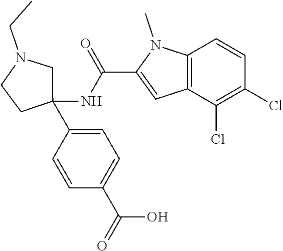 [SMILES: CCN1CCC(NC(=O)c2cc3c(Cl)c(Cl)ccc3n2C)(c2ccc(C(=O)O)cc2)C1] |
| 127 | (±)-4-[3-[(4,5-Dichloro-1- methyl-indole-2- carbonyl)amino]-oxetan-3- yl]benzoic acid | 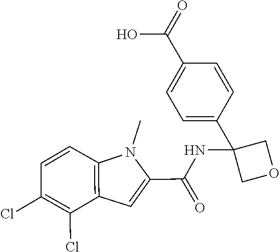 [SMILES: Cn1c(C(=O)NC2(c3ccc(C(=O)O)cc3)COC2)cc2c(Cl)c(Cl)ccc21] |
| 128 | (±)-4,5-Dichloro-1-methyl-N- [3-[4-(1H-tetrazol-5- yl)phenyl]tetrahydro-furan-3- yl]indole-2-carboxamide | 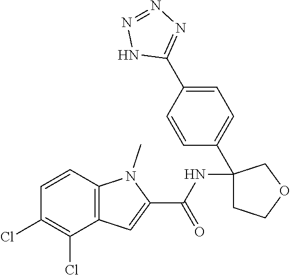 [SMILES: Cn1c(C(=O)NC2(c3ccc(-c4nnn[nH]4)cc3)CCOC2)cc2c(Cl)c(Cl)ccc21] |
| 129 | (±)-2-[4-[3-[(4,5-Dichloro-1- methyl-indole-2- carbonyl)amino] tetrahydrofuran- 3-yl]phenyl] acetic acid | 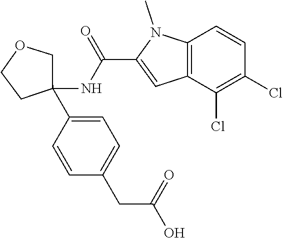 [SMILES: Cn1c(C(=O)NC2(c3ccc(CC(=O)O)cc3)CCOC2)cc2c(Cl)c(Cl)ccc21] |
| 130 | (±)-4-[3-[(4,5-dichloro-1- methyl-indole-2- carbonyl)amino]-1-methyl- pyrrolidin-3-yl] benzoic acid | 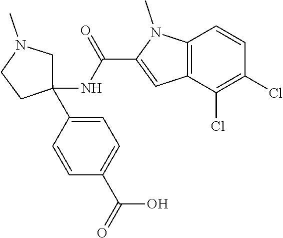 [SMILES: CN1CCC(NC(=O)c2cc3c(Cl)c(Cl)ccc3n2C)(c2ccc(C(=O)O)cc2)C1] |
| 131 | (±)-N-[3-(4- Carbamoylphenyl) tetrahydrofuran- 3-yl]-4,5-dichloro-1- methyl-indole-2- carboxamide | 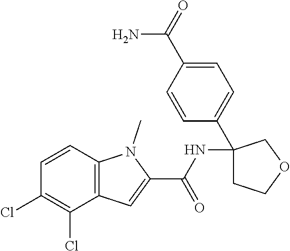 [SMILES: Cn1c(C(=O)NC2(c3ccc(C(N)=O)cc3)CCOC2)cc2c(Cl)c(Cl)ccc21] |
| 132 | (R)-4-[(3)-3-[(4,5-dichloro-1- methyl-indole-2-carbonyl)- amino]-tetrahydrofuran-3- yl]benzoic acid | 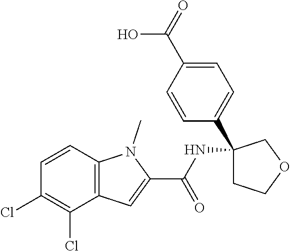 [SMILES: Cn1c(C(=O)N[C@@]2(c3ccc(C(=O)O)cc3)CCOC2)cc2c(Cl)c(Cl)ccc21] |
| 133 | (S)-4-[(3)-3-[(4,5-Dichloro-1- methyl-indole-2- carbonyl)amino]- tetrahydrofuran-3-yl]benzoic acid | 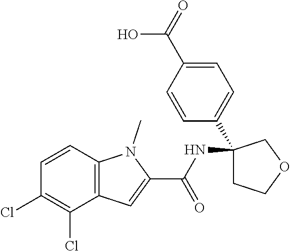 [SMILES: Cn1c(C(=O)N[C@]2(c3ccc(C(=O)O)cc3)CCOC2)cc2c(Cl)c(Cl)ccc21] |
| 134 | (±)-4,5-dichloro-N-[3-(4- cyanophenyl)tetrahydrofuran- 3-yl]-1-methyl-indole-2- carboxamide | 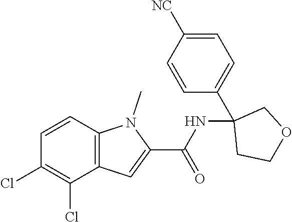 [SMILES: Cn1c(C(=O)NC2(c3ccc(C#N)cc3)CCOC2)cc2c(Cl)c(Cl)ccc21] |
| 135 | (±)-N-[3-(4- Bromophenyl)tetrahydrofuran- 3-yl]-4,5-dichloro-1-methyl- indole-2-carboxamide | 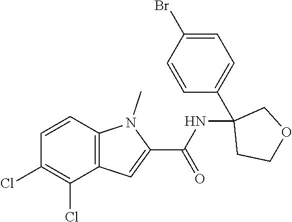 [SMILES: Cn1c(C(=O)NC2(c3ccc(Br)cc3)CCOC2)cc2c(Cl)c(Cl)ccc21] |
| 136 | (±)-4-[3-[(4,5-Dichloro-1- methyl-indole-2- carbonyl)amino] tetrahydrofuran- 3-yl]benzoic acid | 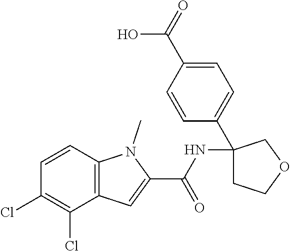 [SMILES: Cn1c(C(=O)NC2(c3ccc(C(=O)O)cc3)CCOC2)cc2c(Cl)c(Cl)ccc21] |
| 137 | (±)-4-[3-[(4,5-Dichloro-1- methyl-indole-2- carbonyl)amino] tetrahydrofuran- 3-yl]benzoic acid | 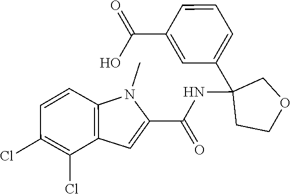 [SMILES: Cn1c(C(=O)NC2(c3cccc(C(=O)O)c3)CCOC2)cc2c(Cl)c(Cl)ccc21] |
| 138 | (±)-4,5-Dichloro-N-[3-[3- (hydroxymethyl)phenyl]-1- (methylcarbamoyl- sulfamoyl)-3-piperidyl]-1- methyl-indole-2-carboxamide | 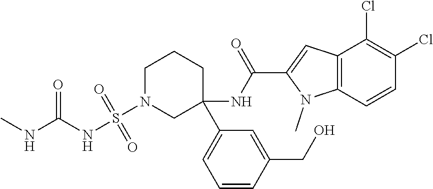 [SMILES: CNC(=O)NS(=O)(=O)N1CCCC(NC(=O)c2cc3c(Cl)c(Cl)ccc3n2C)(c2cccc(CO)c2)C1] |
| 139 | (±)-3-[3-[(4,5-Dichloro-1- methyl-indole-2- carbonyl)amino]-1- (methylcarbamoylsulfamoyl)- 3-piperidyl]benzoic acid | 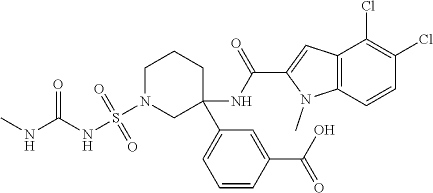 [SMILES: CNC(=O)NS(=O)(=O)N1CCCC(NC(=O)c2cc3c(Cl)c(Cl)ccc3n2C)(c2cccc(C(=O)O)c2)C1] |
| 140 | (±)-4-(3-(4,5-Dichloro-1- methyl-1H-indole-2- carboxamido)-1-(N- (methylcarbamoyl) sulfamoyl)piperidin-3- yl)benzoic acid | 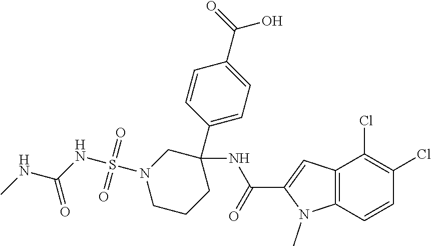 [SMILES: CNC(=O)NS(=O)(=O)N1CCCC(NC(=O)c2cc3c(Cl)c(Cl)ccc3n2C)(c2ccc(C(=O)O)cc2)C1] |
| 141 | (±)-N-[3-(3- Carbamoylphenyl) tetrahydrofuran- 3-yl]-4,5-dichloro-1- methyl-indole-2-carboxamide | 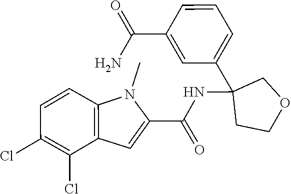 [SMILES: Cn1c(C(=O)NC2(c3cccc(C(N)=O)c3)CCOC2)cc2c(Cl)c(Cl)ccc21] |
| 142 | (±)-4,5-Dichloro-N-[3-(3- cyanophenyl)-1- (methylcarbamoylsulfamoyl)- 3-piperidyl]-1-methyl-indole- 2-carboxamide | 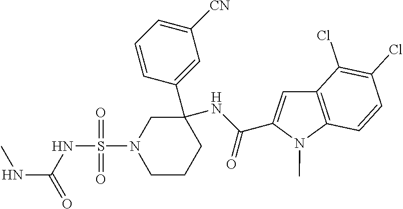 [SMILES: CNC(=O)NS(=O)(=O)N1CCCC(NC(=O)c2cc3c(Cl)c(Cl)ccc3n2C)(c2cccc(C#N)c2)C1] |
| 143 | (±)-Dichloro-N-[3-(3- cyanophenyl)tetrahydrofuran- 3-yl]-1-methyl-indole-2- carboxamide | 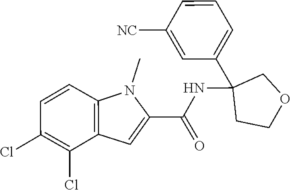 [SMILES: Cn1c(C(=O)NC2(c3cccc(C#N)c3)CCOC2)cc2c(Cl)c(Cl)ccc21] |
| 144 | 4-[1-(Acetylsulfamoyl)-4- [(4,5-dichloro-1-methyl- indole-2-carbonyl)amino]-4- piperidyl]benzoic acid | 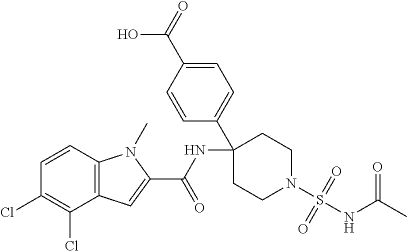 [SMILES: CC(=O)NS(=O)(=O)N1CCC(NC(=O)c2cc3c(Cl)c(Cl)ccc3n2C)(c2ccc(C(=O)O)cc2)CC1] |
| 145 | N-(1-(N-Acetylsulfamoyl)-4- (3-aminophenyl)piperidin-4- yl)-4,5-dichloro-1-methyl- 1H-indole-2-carboxamide | 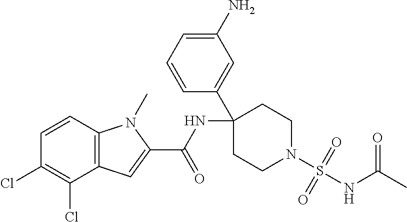 [SMILES: CC(=O)NS(=O)(=O)N1CCC(NC(=O)c2cc3c(Cl)c(Cl)ccc3n2C)(c2cccc(N)c2)CC1] |
| 146 | (±)-4,5-dichloro-1-methyl-N- [1- (methylcarbamoylsulfamoyl)- 3-phenyl-3-piperidyl]indole- 2-carboxamide | 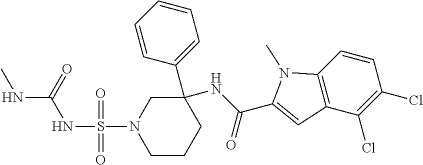 [SMILES: CNC(=O)NS(=O)(=O)N1CCCC(NC(=O)c2cc3c(Cl)c(Cl)ccc3n2C)(c2ccccc2)C1] |
| 147 | (±)-4,5-Dichloro-N-[3-(3- cyanophenyl)-1- (methylcarbamoylsulfamoyl)- 3-piperidyl]-1-methyl-indole- 2-carboxamide | 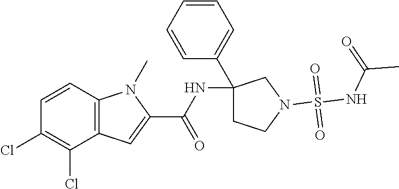 [SMILES: CC(=O)NS(=O)(=O)N1CCC(NC(=O)c2cc3c(Cl)c(Cl)ccc3n2C)(c2ccccc2)C1] |
| 148 | N-(1-(N-acetylsulfamoyl)-3- phenylazetidin-3-yl)-4,5- dichloro-1-methyl-1H-indole- 2-carboxamide | 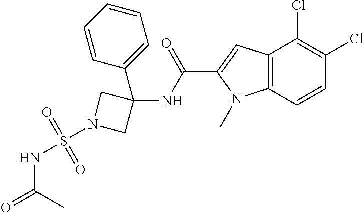 [SMILES: CC(=O)NS(=O)(=O)N1CC(NC(=O)c2cc3c(Cl)c(Cl)ccc3n2C)(c2ccccc2)C1] |
| 149 | N-(1-(N-acetylsulfamoyl)-4- (3- (hydroxymethyl) phenyl)piperidin- 4-yl)-4,5-dichloro-1- methyl-1H-indole-2- carboxamide | 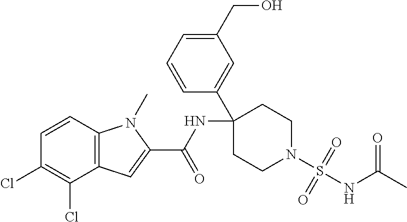 [SMILES: CC(=O)NS(=O)(=O)N1CCC(NC(=O)c2cc3c(Cl)c(Cl)ccc3n2C)(c2cccc(CO)c2)CC1] |
| 150 | N-(4-(3-Acetamidophenyl)-1- acetylpiperidin-4-yl)-4,5- dichloro-1-methyl-1H- indole-2-carboxamide | 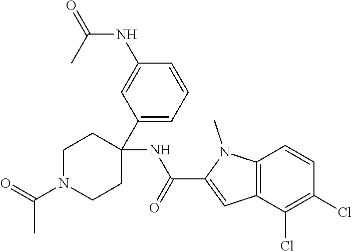 [SMILES: CC(=O)Nc1cccc(C2(NC(=O)c3cc4c(Cl)c(Cl)ccc4n3C)CCN(C(C)=O)CC2)c1] |
| | 3-(1-(N-acetylsulfamoyl)-4- (4,5-dichloro-1-methyl-1H- indole-2-carboxamido) piperidin-4-yl)benzoic acid | 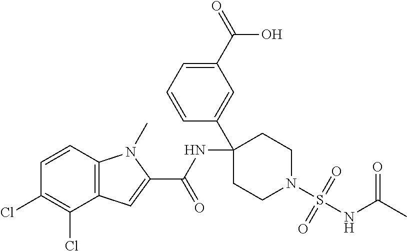 [SMILES: CC(=O)NS(=O)(=O)N1CCC(NC(=O)c2cc3c(Cl)c(Cl)ccc3n2C)(c2cccc(C(=O)O)c2)CC1] |
| 152 | 4,5-Dichloro-1-methyl-N-[4- phenyl-1-(pyrazin-2- ylsulfamoyl)-4- piperidyl]indole-2- carboxamide | 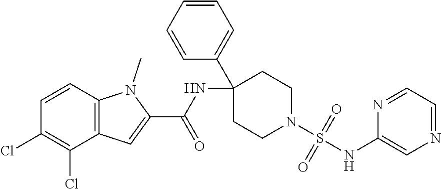 [SMILES: Cn1c(C(=O)NC2(c3ccccc3)CCN(S(=O)(=O)Nc3cnccn3)CC2)cc2c(Cl)c(Cl)ccc21] |
| 153 | N-(4-(3-acetamidophenyl)-1- (N-acetylsulfamoyl)piperidin- 4-yl)-4,5-dichloro-1-methyl- 1H-indole-2-carboxamide | 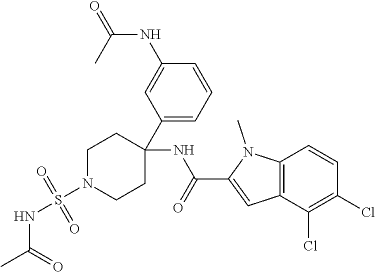 [SMILES: CC(=O)Nc1cccc(C2(NC(=O)c3cc4c(Cl)c(Cl)ccc4n3C)CCN(S(=O)(=O)NC(C)=O)CC2)c1] |
| 154 | N-(1-(N-acetylsulfamoyl)-4- (3-cyanophenyl)piperidin-4- yl)-4,5-dichloro-1-methyl- 1H-indole-2-carboxamide | 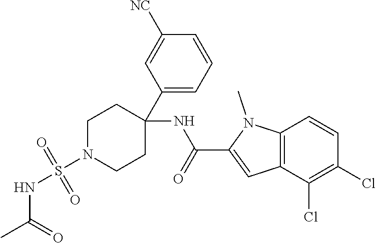 [SMILES: CC(=O)NS(=O)(=O)N1CCC(NC(=O)c2cc3c(Cl)c(Cl)ccc3n2C)(c2cccc(C#N)c2)CC1] |
| 155 | N-(1-(N-acetylsulamoyl)-4- phenylpiperidin-4-yl)-4,5- dichloro-1-methyl-1H-indole- 2-carboxamide | 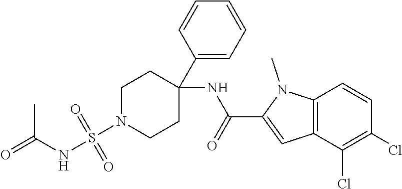 [SMILES: CC(=O)NS(=O)(=O)N1CCC(NC(=O)c2cc3c(Cl)c(Cl)ccc3n2C)(c2ccccc2)CC1] |
| 156 | (±)-ethoxy-1-methyl-1H- indole-2-amido)oxetan-3- yl]phenyl}-2- cyclohexylacetic acid | 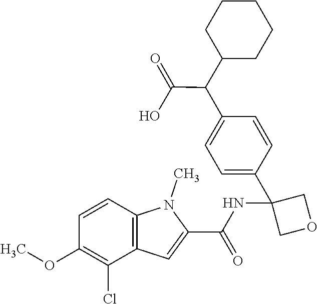 [SMILES: COc1ccc2c(cc(C(=O)NC3(c4ccc(C(C(=O)O)C5CCCCC5)cc4)COC3)n2C)c1Cl] |
| 157 | 2R)-2-[4-[3-[(4-chloro-5- methoxy-1-methyl-indole-2- carbonyl)amino]oxetan-3- yl]phenyl]-3-methyl-butanoic acid | 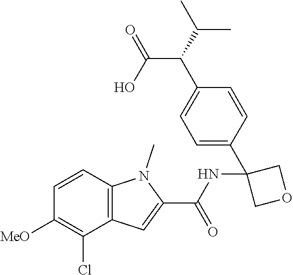 [SMILES: COc1ccc2c(cc(C(=O)NC3(c4ccc([C@H](C(=O)O)C(C)C)cc4)COC3)n2C)c1Cl] |
| 158 | (2S)-2-[4-[3-[(4-chloro-5- methoxy-1-methyl-indole-2- carbonyl)amino]oxetan-3- yl]phenyl]-3-methyl-butanoic acid | 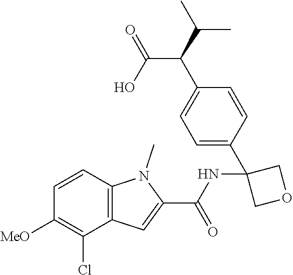 [SMILES: COc1ccc2c(cc(C(=O)NC3(c4ccc([C@@H](C(=O)O)C(C)C)cc4)COC3)n2C)c1Cl] |
| 159 | (±)-2-{4-[3-(4-chloro-5- methoxy-1-methyl-1H-indole- 2-amido)oxetan-3-yl]phenyl}- 2-(piperidin-4-yl)acetic acid | 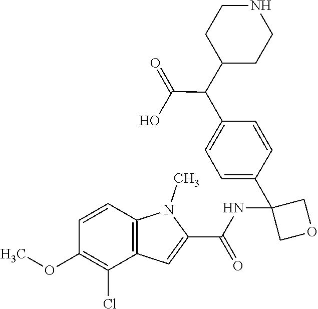 [SMILES: COc1ccc2c(cc(C(=O)NC3(c4ccc(C(C(=O)O)C5CCNCC5)cc4)COC3)n2C)c1Cl] |
| 160 | (±)-2-[4-[3-[(4-Chloro-5- methoxy-1-methyl-indole-2- carbonyl)amino] oxetan-3- yl]phenyl]-2-(4- hydroxycyclohexyl)acetic acid | 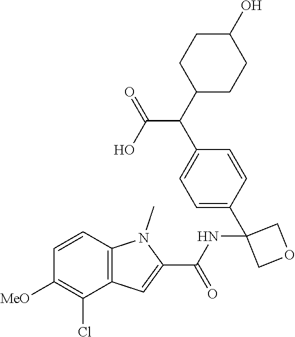 [SMILES: COc1ccc2c(cc(C(=O)NC3(c4ccc(C(C(=O)O)C5CCC(O)CC5)cc4)COC3)n2C)c1Cl] |
| 161 | 4-Chloro-5-methoxy-1- methyl-N-(3-(4-(N- propionylsulfamoyl)phenyl)- oxetan-3-yl)-1H-indole-2- carboxamide | 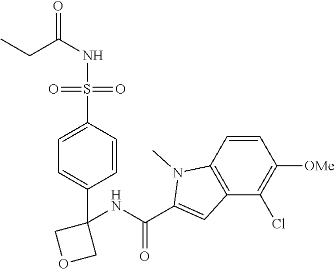 [SMILES: CCC(=O)NS(=O)(=O)c1ccc(C2(NC(=O)c3cc4c(Cl)c(OC)ccc4n3C)COC2)cc1] |
| 162 | 4-Chloro-5-methoxy-1- methyl-N-(3-(4- ((methylsulfonyl)carbamoyl)- phenyl)oxetan-3-yl)-1H- indole-2-carboxamide | 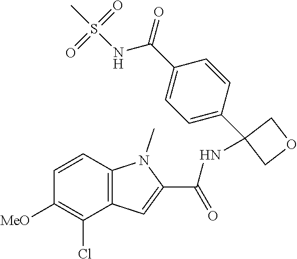 [SMILES: COc1ccc2c(cc(C(=O)NC3(c4ccc(C(=O)NS(C)(=O)=O)cc4)COC3)n2C)c1Cl] |
| 163 | (±)-2-Cyclopropyl-2-(4-(3- (4,5-dichloro-6-methoxy-1- methyl-1H-indole-2- carboxamido) tetrahydrofuran-3- yl)phenyl)acetic acid | 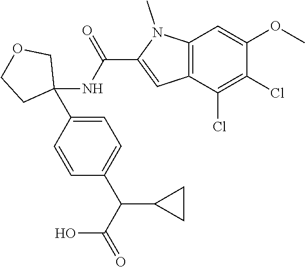 [SMILES: COc1cc2c(cc(C(=O)NC3(c4ccc(C(C(=O)O)C5CC5)cc4)CCOC3)n2C)c(Cl)c1Cl] |
| 164 | 4-chloro-N-{3-[4- (acetamidosulfonyl)phenyl] oxetan-3-yl}-5-methoxy-1- methyl-1H-indole-2- carboxamide | 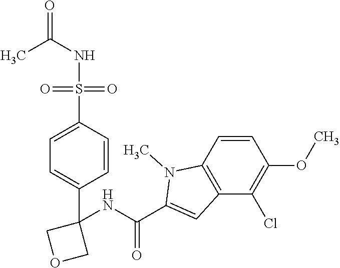 [SMILES: COc1ccc2c(cc(C(=O)NC3(c4ccc(S(=O)(=O)NC(C)=O)cc4)COC3)n2C)c1Cl] |
| 165 | (±)-2-{4-[3-(4-chloro-5- methoxy-1-methyl-1H-indole- 2-amido)oxetan-3-yl]phenyl}- 2-(oxan-4-yl)acetic acid | 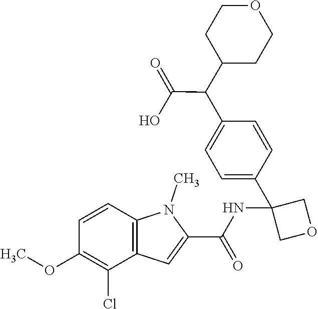 [SMILES: COc1ccc2c(cc(C(=O)NC3(c4ccc(C(C(=O)O)C5CCOCC5)cc4)COC3)n2C)c1Cl] |

It is to be understood that all the single deuterates, enantiomers, diastereoisomers and mixtures thereof, in any proportion, or pharmaceutically acceptable salts and solvates of the compounds of formula (I) are encompassed within the scope of the present invention.

In one preferred embodiment, the present invention relates to at least one of the compounds selected from those listed in the above Table 1 for use in the prevention and/or treatment of fibrosis selected from the group consisting of pulmonary fibrosis, idiopathic pulmonary fibrosis (IPF), chronic hypersensitivity pneumonitis (CHP), hepatic fibrosis, kidney or renal fibrosis, ocular fibrosis, cardiac fibrosis, arterial fibrosis, sarcoidosis and systemic sclerosis, liver fibrosis and Cirrhosis.

In one more preferred embodiment, the present invention relates to at least one of the compounds selected from those listed in the above Table 1 for use in the prevention and/or treatment of IPF.

In an equally more preferred embodiment, the present invention relates to at least one of the compounds selected from those listed in the above Table 1 for use in the prevention and/or treatment of renal fibrosis.

The preparation of compounds described in Table 1 and intermediates of synthesis was already described in WO 2017156165 (Raze Therapeutics), along with the PHGDH Activity Inhibition Data.

In one embodiment, the present invention refers to the Compound 19

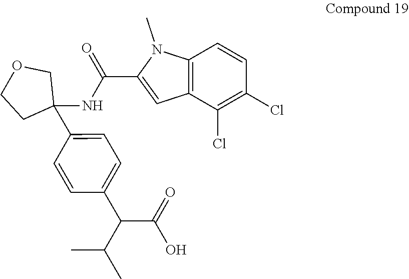 [SMILES: CC(C)C(C(=O)O)c1ccc(C2(NC(=O)c3cc4c(Cl)c(Cl)ccc4n3C)CCOC2)cc1]

for use in the prevention and/or treatment of fibrosis.

In one preferred embodiment, the present invention refers to the Compound 19 for use in the prevention and/or treatment of fibrosis selected from the group consisting of pulmonary fibrosis, idiopathic pulmonary fibrosis (IPF), chronic hypersensitivity pneumonitis (CHP), hepatic fibrosis, kidney or renal fibrosis, ocular fibrosis, cardiac fibrosis, arterial fibrosis, sarcoidosis and systemic sclerosis, liver fibrosis and Cirrhosis.

In a more preferred embodiment, the present invention refers to the Compound 19 for use in the prevention and/or treatment of IPF.

In another embodiment, the present invention refers to the Compound 19a, Diasteroisomer 2 of (±)-2-(4-(3-(4,5-Dichloro-1-methyl-1H-indole-2-carboxamido)tetrahydro-furan-3-yl) phenyl)-3-methylbutanoic acid, for use in the prevention and/or treatment of fibrosis.

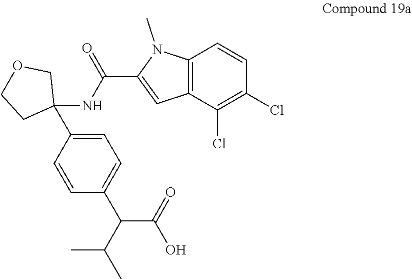 [SMILES: CC(C)C(C(=O)O)c1ccc(C2(NC(=O)c3cc4c(Cl)c(Cl)ccc4n3C)CCOC2)cc1]

In one preferred embodiment, the present invention refers to the Compound 19a for use in the prevention and/or treatment of fibrosis selected from the group consisting of pulmonary fibrosis, idiopathic pulmonary fibrosis (IPF), chronic hypersensitivity pneumonitis (CHP), hepatic fibrosis, kidney or renal fibrosis, ocular fibrosis, cardiac fibrosis, arterial fibrosis, sarcoidosis and systemic sclerosis, liver fibrosis and Cirrhosis.

In a more preferred embodiment, the present invention refers to the Compound 19a for use in the prevention and/or treatment of IPF.

In an equally preferred embodiment, the present invention refers to a Compound 19a for use in the prevention and/or treatment of renal fibrosis.

The compounds of formula (I) are typically administered once or even more, for example twice daily optionally in combination with a dosing regimen wherein a number of doses are administered at varying intervals of time for a given period of time, according to the skilled person knowledge.

The dosages of the compounds of the invention may depend upon a variety of factors including among others the particular disease to be treated, the severity of the symptoms, the route of administration and the like.

In another embodiment, the invention relates to compounds of Formula (I), or a pharmaceutically acceptable salt thereof, for use for the prevention and/or treatment of fibrosis whereby said compounds is administered for days, weeks, months or years, including indefinitely.

In one aspect, the present invention also refers to a pharmaceutical composition comprising a compound of formula (I) or a pharmaceutically acceptable salt thereof, in admixture with at least one or more pharmaceutically acceptable carrier or excipient for use in the prevention and/or treatment of fibrosis.

In one embodiment, the invention refers to a pharmaceutical composition of compounds of formula (I) or a pharmaceutically acceptable salt thereof, for use in the prevention and/or treatment of fibrosis in admixture with one or more pharmaceutically acceptable carrier or excipient for example those described in Remington's Pharmaceutical Sciences Handbook, XVII Ed., Mack Pub., N.Y., U.S.A, Administration of the compounds of the invention and their pharmaceutical compositions may be accomplished according to patient needs, for example, orally, nasally, parenterally (subcutaneously, intravenously, intramuscularly, intradermally and by infusion) and by inhalation.

Preferably, the compounds of the present invention for use in the prevention and/or treatment of fibrosis are administered orally or by inhalation.

In one preferred embodiment, the pharmaceutical composition comprising the compound of formula (I) is a solid oral dosage form such as tablets, gel caps, capsules, caplets, granules, lozenges and bulk powders.

The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In a further embodiment, the pharmaceutical composition comprising a compound of formula (I) for use in the prevention and/or treatment of fibrosis is a liquid oral dosage forms such as aqueous and non-aqueous solutions, emulsions, suspensions, syrups. Such liquid dosage forms can also contain suitable known inert diluents such as water and suitable known excipients such as preservatives, wetting agents, sweeteners, flavorants, as well as agents for emulsifying and/or suspending the compounds of the invention.

In a further embodiment, the pharmaceutical composition comprising the compound of formula (I) for use in the prevention and/or treatment of fibrosis is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges, or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers, and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

In one aspect, the invention related to a device comprising a pharmaceutical composition comprising a compound of Formula (I) for use in the prevention and/or treatment of fibrosis according to the invention, in form of a single- or multi-dose dry powder inhaler or a metered dose inhaler.

The compounds of the invention for use in the prevention and/or treatment of fibrosis can be administered as the sole active agent or in combination with other pharmaceutical active ingredients.

In a further aspect, the present invention relates to the use of compounds of Formula (I), or a pharmaceutically acceptable salt thereof, for the prevention and/or treatment of fibrosis.

In one embodiment, the present invention relates to the use of compounds of Formula (I), or a pharmaceutically acceptable salt thereof, for the prevention and/or treatment of fibrosis selected from the group consisting of pulmonary fibrosis, idiopathic pulmonary fibrosis (IPF), chronic hypersensitivity pneumonitis (CHP), hepatic fibrosis, kidney or renal fibrosis, ocular fibrosis, cardiac fibrosis, arterial fibrosis, sarcoidosis and systemic sclerosis, liver fibrosis and Cirrhosis.

In one preferred embodiment, the present invention relates to the use of compounds of Formula (I), or a pharmaceutically acceptable salt thereof, for the prevention and/or treatment of IPF.

In one preferred embodiment, the present invention relates to the use of compounds of Formula (I), or a pharmaceutically acceptable salt thereof, for the prevention and/or treatment of renal fibrosis.

In a more preferred embodiment, the present invention relates to the use of Compound 19 for the prevention and/or treatment of IPF.

In another preferred embodiment, the present invention relates to the use of Compound 19a for the prevention and/or treatment of IPF.

In a further aspect, the present invention relates to a method for the prevention and/or treatment of fibrosis administering compounds of Formula (I), or a pharmaceutically acceptable salt thereof.

In one embodiment, the present invention relates to a method for the prevention and/or treatment of fibrosis selected from the group consisting of pulmonary fibrosis, idiopathic pulmonary fibrosis (IPF), chronic hypersensitivity pneumonitis (CHP), hepatic fibrosis, kidney or renal fibrosis, ocular fibrosis, cardiac fibrosis, arterial fibrosis, sarcoidosis and systemic sclerosis, liver fibrosis and Cirrhosis, administering compounds of Formula (I), or a pharmaceutically acceptable salt thereof.

In one preferred embodiment, the present invention relates to a method for the prevention and/or treatment of IPF administering compounds of Formula (I), or a pharmaceutically acceptable salt thereof.

In an equally preferred embodiment, the present invention relates to a method for the prevention and/or treatment of renal fibrosis administering compounds of Formula (I), or a pharmaceutically acceptable salt thereof.

In a more preferred embodiment, the present invention relates to a method for the prevention and/or treatment of IPF administering Compound 19, or a pharmaceutically acceptable salt thereof.

In another preferred embodiment, the present invention relates to a method for the prevention and/or treatment of IPF administering Compound 19a, or a pharmaceutically acceptable salt thereof.

All preferred groups or embodiments described above for compounds of formula (I) for use in the prevention and/or treatment of fibrosis may be combined among each other and apply as well mutatis mutandis.

The various aspects of the invention described in this application are illustrated by the following compounds which are not meant to limit the invention in any way.

### PREPARATIONS OF COMPOUNDS

All the compounds of the present invention are synthetized as reported in WO 2017156165, except for Compound 19a, 19b, 19c, 19d, which are obtained from Compound 19 as following.

Chemical Names of the compounds were generated with Structure To Name Enterprise 10.0 Cambridge Software or are common chemical names. All reagents, for which the synthesis is not described in the experimental part, are either commercially available, or are known compounds or may be formed from known compounds by known methods by a person skilled in the art.

In the procedures that follow, some of the starting materials are identified through an “Intermediate” or “Compound” number with indications on step number. This is provided merely for assistance to the skilled chemist.

### Abbreviations

SFC=supercritical fluid chromatography; t_{R}=retention time; iPrNH₂=Isopropylamine;
TMS=tetramethylsilane NMR characterization:

¹H NMR spectra were recorded on Varian MR-400 spectrometer operating at 400 MHZ (proton frequency), equipped with: a self-shielded Z-gradient coil 5 mm 1H/nX broadband probe head for reverse detection, deuterium digital lock channel unit, quadrature digital detection unit with transmitter offset frequency shift.

Alternatively, the NMR Spectra were Performed on a Bruker AVANCE III HD 600 spectrometer operating at 600 MHz (proton frequency). The spectrometer is equipped with a 5 mm TCI INVERSE TRIPLE RESONACE CRYOPROBE H-C/N-D-0.5-Z ATMA. The probe is fitted with an actively shielded single axis Z-gradient and allows simultaneous decoupling on multiple X-nuclei such as 13C and 15N as well as automatic tuning and matching. Standard sample temperature range is comprised between 0° C. and 80° C.

Chemical shifts are reported as 6 values in ppm relative to tetramethylsilane (TMS) as an internal standard. Coupling constants (J values) are given in hertz (Hz) and multiplicities are reported using the following abbreviation (s=singlet, d=doublet, t=triplet, quin=quintet, m=multiplet, br=broad).

In some cases, signals NH from amide bond or OH from acid bond (Exchangeable protons) are not visible. In a few cases, some signals could be hidden under the signal of water or under the signal of DMSO or other residual solvents.

### LC/UV/MS Analytical Methods

LC/MS retention times are estimated to be affected by an experimental error of ±0.5 min.

Method 1: Acquity CSH C18 column 50 mm×2.1 mm 1.7 μm, maintained at 40° C.; Mobile Phase: Eluent B (ACN/water 95:5+0.05% HCOOH) in Eluent A (water/ACN 95:5+0.05% HCOOH) from 1% to 99.9% within 1.5 min. Flow rate: 1 mL/min. Wavelength: 210-400 nm DAD.

UPLC+Waters PDA+Waters QDA.

### Chiral Supercritical Fluid Chromatography (SFC) Separation Protocol

The diastereomeric separation of compounds was achieved by Supercritical Fluid Chromatography (SFC) using a Waters Prep SFC200 system (P200 CO2 pump, 2545 modifier pump, 2489 UV/VIS detector). Analysis of two enantiomers was performed from reconstituted final samples.

The Waters 7937 liquid handler acted as fraction collector. Appropriate isocratic methods were selected based on methanol solvent systems under basic conditions. The standard SFC method used was modifier, CO2, 100 mL/min, 100 Bar backpressure, 40° C. column temperature. The modifier used under basic conditions was Isopropylamine (0.5% V/V). The sample solutions were filtered on GHP 0.45 um filter. Collected fractions were analysed by Agilent SFC (Agilent 1260). The fractions that contained the desired product were concentrated by vacuum centrifugation.

Method 1: SFC-MS was performed on a Waters Thar Prep100 preparative SFC system, using a Chiralcel OD-H (30 mm×250 mm, 5 um) column with an isocratic run (15:85 MeOH+0.5% iPrNH2:CO₂), Flow Rate 100 mL/min, BPR 100 BarG, Detector Wavelength 220 nm, Injection Volume 150 mL, Stacking interval 28 min., elution time 60 min., 40° C. column temperature.

Method 2: SFC-MS was performed on a Waters Thar Prep100 preparative SFC system, using a Chiralpak IC OD-H (30 mm×250 mm, Sum) column with an isocratic run (30:70 MeOH+0.5% iPrNH2:CO₂), Flow Rate 100 mL/min, BPR 100 BarG, Detector Wavelength 220 nm, Injection Volume 25 mL, Stacking interval 4.38 min., elution time 12 min., 40° C. column temperature.

### Supercritical Fluid Chromatography—Mass Spectrometry Analytical Conditions

Method 3: SFC-MS was performed on a SFC Agilent system using a Chiralcel OD (4.6 mm×250 mm, Sum) column with an isocratic run (20:80 MeOH+0.5% iPrNH2:CO₂), Flow Rate 2.4 mL/min, BPR 104 BarG, Detector Wavelength 210 nm, Injection Volume 50 μL (1 mg/mL in MeOH), 40° C. column temperature.

Single diastereoisomers from Compound 19 were obtained by two subsequent preparative chiral SFC.

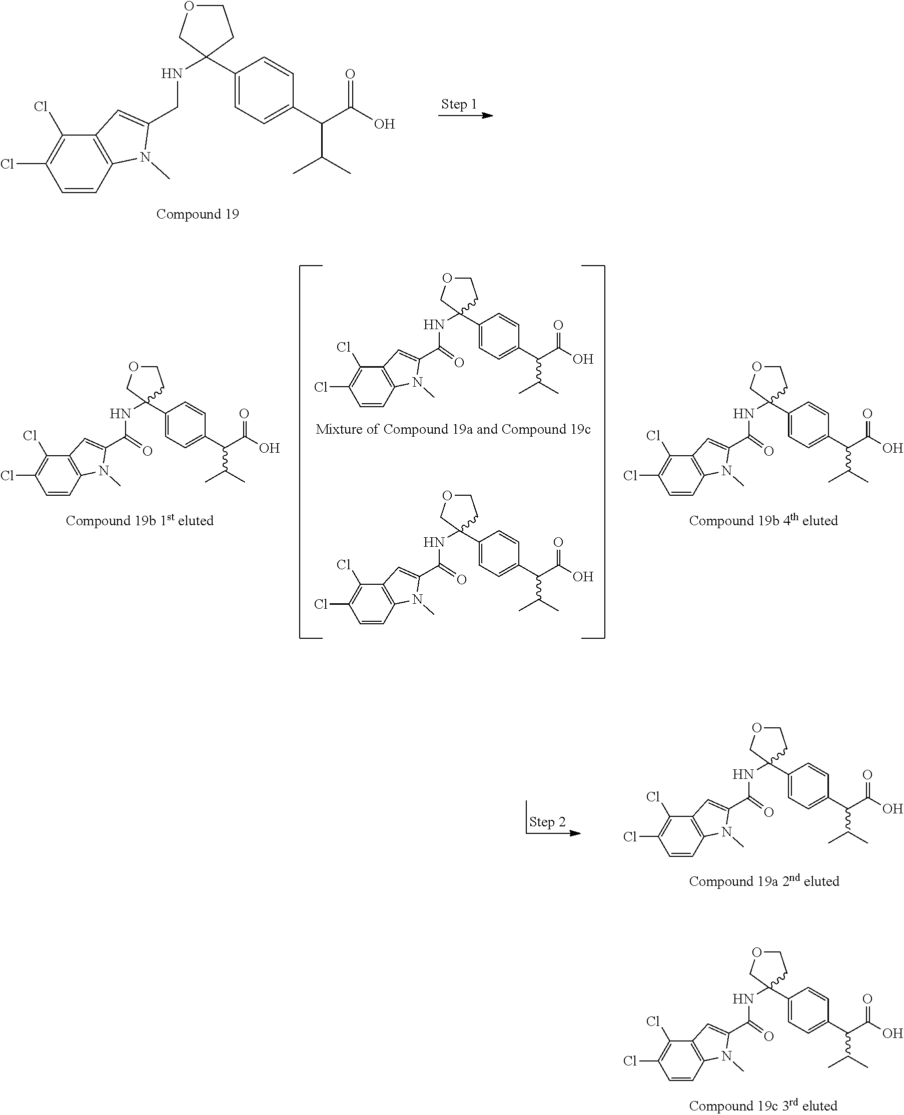

### Compound 19b and Compound 19d: 1 Eluted Diastereoisomer and 4 Eluted Diastereoisomer of 2-(4-(3-(4,5-Dichloro-1-methyl-1H-indole-2-carboxamido)tetrahydro-furan-3-yl) phenyl)-3-methylbutanoic acid

A racemic mixture of Compound 19 (2.5 g, 5.1 mmol), was dissolved in 150 mL of MeGH+0.5p iPrNH2, and was separated by preparative SFC, using Method 1, to give the desired products, as iPrNH2 salts. The stoichiometric ratio iPrNH2:acid observed in ¹H NMR spectra varied from 1:1 to 0.5:1.
1^{st} eluted diastereoisomer (Compound 19b): 491 mg, 19.6%.
4^{st} eluted diastereoisomer (Compound 19dl: 466 m, 18.6%.

A mixture of 2^{nd} and 3^{rd} eluted diastereoisomer: 1.07 g, 42.8% .

### Compound 19a and Compound 19c 2^{st} Eluted Diastereoisomer and 3 Eluted Diastereoisomer of 2-(4-(3-(4,5-Dichloro-1-methyl-1H-indole-2-carboxamido)tetrahydro-furan-3-yl) phenyl)-3-methylbutanoic acid

The mixture of 2^{nd} and 3^{rd} eluted isomers (1.07 g) was dissolved in 25 mL MeOH+0.5% iPrNH2, and was separated by preparative SFC, using Method 2, to give the desired products, as iPrNH2 salts.
2^{st} eluted diastereoisomer (Compound 19a): 374 mg, 35%.
3^{st} eluted diastereoisomer (Compound 19c): 459 mg, 42.9%.

| | Chiral | |
|---|---|---|
| | Analysis | |
| Compound | (Method 3) | LC-MS and ¹H NMR |
| 19b | t_{R} = | LC-MS (ESI, m/z): method 1, t_{R} = 5.4 min, |
| | 10.25 min, | m/z (M + 1) = 489.18 |
| | ee 100% | ¹H NMR (600 MHz, DMSO-d6) δ ppm 9.11 |
| | | (s, 1 H) 7.60 (d, J = 8.85 Hz, 1 H) 7.45 (d, |
| | | J = 8.85 Hz, 1 H) 7.41 (s, 1 H) 7.30 (m, J = |
| | | 8.33 Hz, 2 H) 7.24 (m, J = 8.46 Hz, 2 H) |
| | | 4.28 (d, J = 9.10 Hz, 1 H) 4.08 (d, J = 9.10 |
| | | Hz, 1 H) 3.92-3.95 (m, 2 H) 3.91 (s, 3 H) |
| | | 3.02 (dquin, J = 12.61, 6.34 Hz, 0.5 H) 2.82 |
| | | (br d, J = 11.28 Hz, 1 H) 2.76-2.80 (m, 1 H) |
| | | 2.31 (dt, J = 12.92, 8.03 Hz, 1 H) 2.09-2.17 |
| | | (m, 1 H) 1.00 (d, J = 6.28 Hz, 3 H) 0.96 (d, |
| | | J = 6.41 Hz, 3 H) 0.57 (d, J = 6.67 Hz, 3 |
| | | H). |
| 19a | t_{R} = | LC-MS (ESI, m/z): method 1, t_{R} = 5.4 min, |
| | 12.97 min, | m/z (M + 1) = 489.18 |
| | ee 98% | ¹H NMR (400 MHz, DMSO-d6) δ ppm 9.07 |
| | | (s, 1 H) 7.55 (d, J = 8.33 Hz, 1 H) 7.41 (d, |
| | | J = 8.99 Hz, 1 H) 7.37 (s, 1 H) 7.26-7.30 |
| | | (m, 2 H) 7.21 (d, J = 8.33 Hz, 2 H) 4.24 (d, |
| | | J = 8.99 Hz, 1 H) 4.05 (d, J = 9.21 Hz, 1 H) |
| | | 3.87-3.92 (m, 2 H) 3.87 (s, 3 H) 3.03 (dt, J = |
| | | 12.72, 6.36 Hz, 1 H) 2.85 (d, J = 10.30 Hz, |
| | | 1 H) 2.74 (dt, J = 12.72, 6.14 Hz, 1 H) 2.27 |
| | | (dt, J = 12.93, 8.00 Hz, 1 H) 2.05-2.18 (m, 1 |
| | | H) 1.00 (d, J = 6.36 Hz, 6 H) 0.93 (d, J = |
| | | 6.58 Hz, 3 H) 0.55 (d, J = 6.80 Hz, 3 H). |
| 19c | t_{R} = | LC-MS (ESI, m/z): method 1, t_{R} = 5.4 min, |
| | 14.34 min, | m/z (M + 1) = 489.18 |
| | ee 98% | ¹H NMR (400 MHz, DMSO-d6) δ ppm 9.06 |
| | | (s, 1 H) 7.55 (d, J = 8.86 Hz, 1 H) 7.41 (d, |
| | | J = 8.99 Hz, 1 H) 7.37 (s, 1 H) 7.28 (m, J = |
| | | 8.55 Hz, 2 H) 7.21 (m, J = 8.55 Hz, 2 H) |
| | | 4.24 (d, J = 9.21 Hz, 1 H) 4.05 (d, J = 8.99 |
| | | Hz, 1 H) 3.87-3.92 (m, 2 H) 3.87 (s, 3 H) |
| | | 2.96-3.05 (m, 1 H) 2.85 (d, J = 10.52 Hz, 1 |
| | | H) 2.74 (dt, J = 12.61, 6.19 Hz, 1 H) 2.27 |
| | | (dt, J = 13.10, 8.03 Hz, 1 H) 2.06-2.17 (m, 1 |
| | | H) 0.99 (d, J = 6.36 Hz, 6 H) 0.93 (d, J = |
| | | 6.58 Hz, 3 H) 0.55 (d, J = 6.58 Hz, 3 H). |
| 19d | t_{R} = | LC-MS (ESI, m/z): method 1, t_{R} = 5.4 min, |
| | 16.07 min, | m/z (M + 1) = 489.18 |
| | ee 98% | ¹H NMR (400 MHz, DMSO-d6) δ ppm 9.07 |
| | | (s, 1 H) 7.55 (d, J = 8.33 Hz, 1 H) 7.41 (d, |
| | | J = 8.77 Hz, 1 H) 7.37 (s, 1 H) 7.29 (m, J = |
| | | 8.33 Hz, 2 H) 7.21 (m, J = 8.33 Hz, 2 H) |
| | | 4.24 (d, J = 8.99 Hz, 1 H) 4.05 (d, J = 8.99 |
| | | Hz, 1 H) 3.87-3.92 (m, 2 H) 3.87 (s, 3 H) |
| | | 2.99-3.07 (m, 1 H) 2.86 (d, J = 10.30 Hz, 1 |
| | | H) 2.69-2.80 (m, 1 H) 2.27 (dt, J = 12.83, |
| | | 8.06 Hz, 1 H) 2.06-2.16 (m, 1 H) 1.00 (d, |
| | | J = 6.36 Hz, 6 H) 0.93 (d, J = 6.58 Hz, 3 H) |
| | | 0.55 (d, J = 6.58 Hz, 3 H). |

### Pharmacological Activity of the Compounds of the Invention

### In Vitro Assay

### TGFb-Induced Profibrotic Markers Release and Serum-Induced Proliferation

The antifibrotic activity of compounds of the present invention has been monitored measuring the expression of fibrotic mediators, collagen I (Col-I) and alpha smooth muscle actin (α-SMA) after treatment in lung cells with TGF-β in comparison with Nintedanib (see Frank Hilberg et al, Cancer Res 2008 Jun. 15; 68(12):4774-82), and allosteric inhibitor NCT-503 (see Hamanaka et al., Am J Respir Cell Mol Biol. 2018 May; 58(5):585-593).

### Method

Human normal lung fibroblasts (NHLFs) were purchased from Lonza (Basel, Switzerland). NHFLs were cultured in Eagle's Minimum Essential medium (EMEM) w/o serine and glycine, supplemented with 10% Fetal Bovine Serum (FBS), antibiotics (50 U/ml penicillin and 0.05 mg/ml streptomycin) and 2 mM L-glutamine, in an atmosphere of 95% air and 5% CO2 at 37° C. NHLFs cultures were used between passage 1 and 9. Before all the experiments, cells were incubated overnight in serum-free medium.

NHLFs were seeded in 12-well plates at cultured until 70% of confluency. To examine the expression of fibrotic markers, cells were treated with different concentrations of Compounds and NCT-503 in EMEM medium for 1 h followed by stimulation with TGF-β for 48 h at 37° C., 5% CO₂. Following cell treatments, medium was removed and whole cell extracts were prepared by directly adding to the well the 1× loading buffer (125 mM Tris-HCl pH 6.8, 4% SDS, 0.2% Orange G, 50% glycerol, 2.5% β-mercaptoethanol, Li-cor Biosciences), boiled for 5 min and then electrophoresed on Mini protean TGX gel 4-12% (Biorad). After electrophoresis, proteins were transferred to a nitrocellulose membrane. Immunodetection of Col-I, α-SMA and glyceraldeide 3-phosphate dehydrogenase (GAPDH) were obtained by using mouse anti-CollA1 (Santacruz), mouse anti-α-SMA (sigma) and rabbit anti-GAPDH (Cell Signaling Technology) all diluted at 1:1000 in TBS-tween 0.1%. Secondary antibodies were goat anti rabbit and goat anti mouse IgG conjugated with IRDye800 and IRDye680 probes (Li-cor) respectively, both diluted at 1:15,000 in TBS-tween 0.1% containing 1% of skim milk. Detection and quantification were performed with Odyssey imaging system (Li-cor) using GAPDH as loading control.

### Example 1: Compound 19 in In Vitro Assays

The antiproliferative activity of compounds of the present invention has been monitored measuring the incorporation of bromodeoxyuridine (BrdU), a thymidine analog of DNA, in newly synthesized DNA after treatment in lung cells. Briefly, NHLFs were seeded in 96-well and treated with different concentrations of Compound 19 and NCT-503 in EMEM medium for 1 h followed by stimulation with Fetal Bovine Serum (FBS) for 72 h at 37° C., 5% CO₂. After cell treatments, 10 μl/well BrdU labeling solution was added for incorporation in proliferating cells for 2 h at 37° C., 5% CO₂. After removing the culture medium, the cells are fixed, and the DNA is denatured by adding FixDenat followed by incubation with 100 μl/well Anti-BrdU-POD for detection. The reaction product is quantified by measuring the absorbance at the respective wavelength using a scanning multiwell spectrophotometer (ELISA reader).

The potency of the Compound 19 and NCT-503 is provided in Table 2.

Compound activity is expressed as pIC₅₀ (Log of the IC₅₀, compound concentration producing 50% of inhibition of the fibrotic markers expression or of the FBS-induced cell proliferation).

TABLE 2
pIC50 values in in vitro assay

| | Compound 19 | NCT-503 |
|---|---|---|
| TGF-b induced Collagen I (pIC₅₀-C.I.) | 7.5 (8.1-6.9) | |
| TGF-b induced a-sma (% inh. 50 uM) | 97 | 93 |
| FBS induced proliferation (pIC₅₀-C.I.) | 6.2 (6.7-5.7) | NA |

C.I.═Confidence Interval; NA: not active

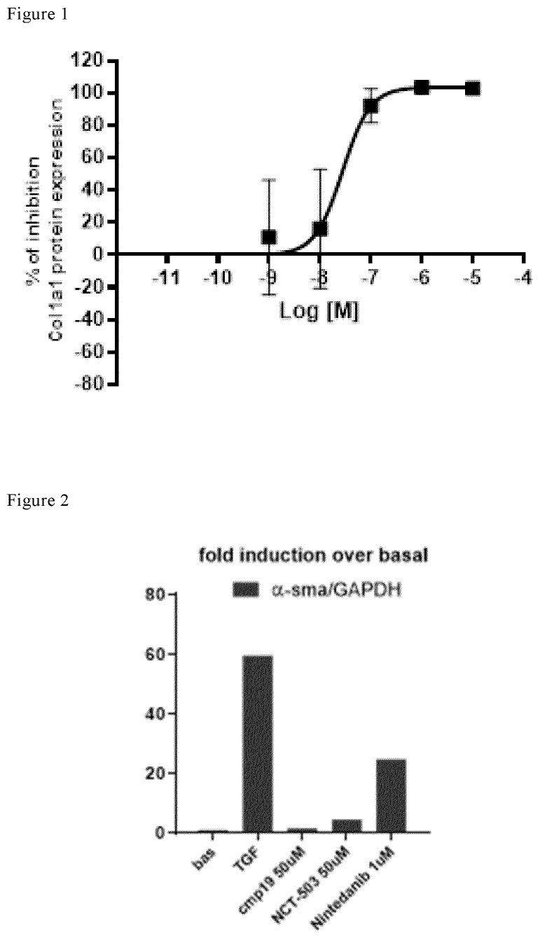

As can be appreciated in FIG. 1, the Compound 19 reduces the fibroblast to myofibroblast transition (FMT) induced by TGF-β as shown by decrease of Collagen protein expression (A).

As shown in FIG. 2, the Compound 19 (50 uM) is able to inhibit the release of and α-SMA in NHLF treated with TGF-β (10 ng/ml) for 48 h in a more efficacious manner than the NCT-503 (50 uM) and even more than the Nintedanib (1 uM).

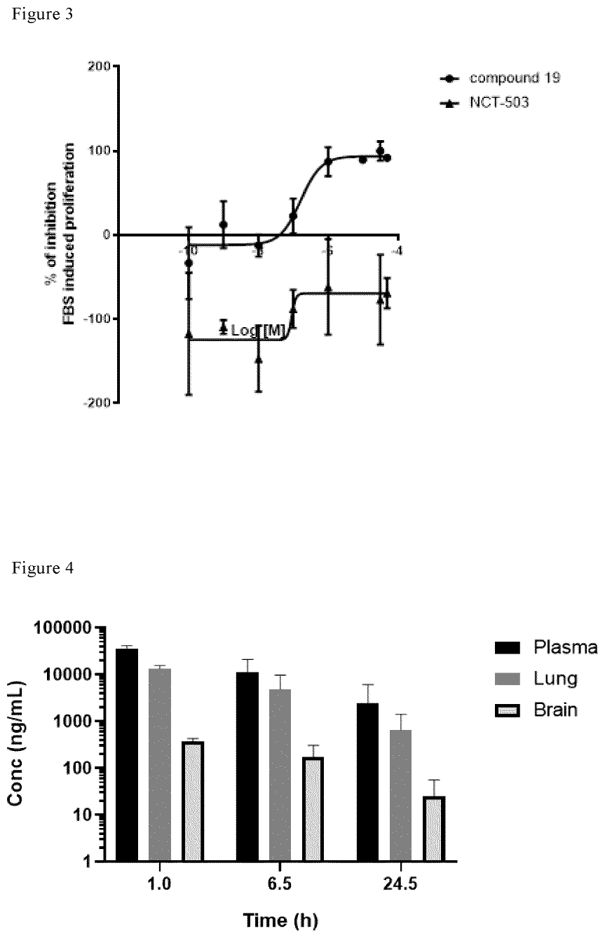

Differently from the NCT-503, the Compound 19 is also able to reduce the cell proliferation induced by serum treatment in NHLFs (72 h), as shown in FIG. 3.

Phenotypic Assay—High Content Screening Optimization of aSMA Staining in NHLF Cells

### Method

NHLF cells (Lonza #CC2512) were seeded as 2000 cells/well in 384-well plates in MEM medium w/o serine and glycine (Sigma #M2279)+1% pen/strep+5 mM L-glutamine +10% FBS and incubated overnight at 37° C., 5% CO₂.

The next day, cells were washed with PBS and starvation MEM medium w/o serine and glycine (0% FBS) was added and cells were incubated overnight at 37° C., 5% CO₂. The day after, cells were pre-incubated with compounds or vehicle (0.3% DMSO) for 1 h. Compounds were tested at 9 consecutive 3-fold dilutions starting from 30 μM. Each compound was tested in duplicate. Cells were further incubated for 72 h at 37° C., 5% CO₂, 95% humidity. After 72 h of incubation, αSMA expression was quantified using immunostaining:
Fixation in 4% paraformaldehyde,
Blocking with 3% BSA, 2% FBS, 0.2% Triton-X in PBS,
Primary ab. anti-αSMA (AbCam #ab7817) diluted 500× in blocking buffer
Anti-mouse secondary ab. AF488 (ThermoFisher #A11029)
% of αSMA positive cells was determined by High Content Imaging (Molecular Device), data analysis was performed using MetaXpress software (Molecular Devices) and % of αSMA positive was further used to calculate % of inhibition for tested compounds. Total cell number was determined by Hoechst staining and % of viability over TGFβ stimulated controls was calculated.

### Fibroblast to Myofibroblast Transition (FMT) In Vitro Assay: Collagen I Detection

### Method

NHLF cells were seeded as 80 000 cells/well in 12-well plates in MEM medium w/o serine and glycine (Sigma #M2279)+1% pen/strep+5 mM L-glutamine+10% FBS and incubated overnight at 37° C., 5% CO₂. The assay was carried out as report above.

### Example 2: Other Compounds in Phenotypic Assay (αSMA and Collagen Detection)

The potencies of the Compounds of this invention and NCT-503 are provided in Table 3. Compound activity is expressed as pIC50 (Log of the IC50, compound concentration producing 50% of inhibition of the fibrotic markers expression).

TABLE 3
pIC50 values in in vitro assay

| Compound | aSMA | Collagen-I |
|---|---|---|
| NCT-503 | >5.0 | |
| 15 | 5.7 | |
| 16 | 6.3 | |
| 19a | 6.6 | 6.9 |
| 19 | 5.9 | |
| 20 | 5.9 | |
| 72 | 5.9 | |

### Results

As reported in the Table 3, all tested compounds, dose-dependently, reduce the expression of aSMA, after 72 hours of incubation. On the contrary, the NCT-503 partially reduces the expression of aSMA. Furthermore, tested compounds dose-dependently inhibit the collagen I deposition, after 72 h of treatment. These findings suggest that de novo synthesis of serine and glycine is required for pulmonary fibrosis; the inhibition of myofibroblast differentiation and collagen deposition is strongly correlated and necessary for the antifibrotic activity of the compounds.

### In Vivo Assays

Effect of the Compounds on the Inhibition of Serine Biosynthesis PK/PD Model: In Vivo [¹³C₆] Glucose Tracing

As PHGDH catalyzes the rate-limiting step of glucose-derived serine synthesis, a pharmacokinetic/pharmacodynamic (PK/PD) experiment aimed to assess the inhibition of the synthesis of ¹³C₃-serine after oral administration of ¹³C₆-glucose was performed in mouse male (C57BL/6J).

### Example 3: Compound 19 in PK/PD Model

### Methods

Animals were fasted overnight before treatment. Following 0.5 h from administration of either Compound 19 at dose of 300 mg/kg or vehicle (0.5% Tween 80, 0.5% Methylcellulose in H2O), ¹³C₆-glucose was administered by oral gavage (2 g/kg, Merck Life Science Srl). Blood, lung, and brain samples were collected at 1 h, 6.5 h and 24.5 h after treatment with Compound 19 or vehicle. At the established time-points, animals were terminally anesthetized by intraperitoneal injection of thiopental (200 mg/kg/10 ml;MSD Animal Health Srl) and all tissues were harvested in less than 5 minutes to preserve the metabolic state and immediately frozen in liquid nitrogen. Blood was withdrawn by cardiac puncture and collected in heparine tubes. Plasma was obtained after centrifugation of blood at 10,000 g for 10 min.

The tissue concentration of ¹³C₃-serine was determined by a liquid chromatography-tandem mass spectrometry (LC/MS/MS) assay. GraphPad Prism (version 8.1.2) was used for drawing graphs and for statistical analysis. Plasma or tissue homogenate samples (tissues were homogenized with a water/acetonitrile 50/50 mixture) were extracted with 3 volumes of acetonitrile. Supernanatant was injected into HPLC system (Agilent 1260 Quaternary Pump equipped with an Agilent 1200 High Performance Autosampler SL; Agilent), using a Kinetex EVO C18 analytical column (100 A, 50×2.1 mm, 2.6 μm; Phenomenex) for the analysis of Compound 19 or an InfinityLab Poroshell 120 Hilic-Z (50×2.1 mm 2.7 μm; Agilent) for the analysis of ¹³C₃-serine. The mobile phases consisted of 0.1% formic acid in water (solvent A) and 0.1% formic acid in acetonitrile (solvent B); chromatographic separation was performed with gradient elution method (see conditions below):
Compound 19 (0.0-0.5 min, 10% B; 0.5-3.0 min, gradient to 90% B; 3.0-5.0 min. 90% B; 5.0-6.0 min, gradient to 10% B; 6.0-10.0 min, 10% B—flow rate: 0.4 mL/min).
S¹³C₃-serine (0.0-0.5 min, 95% B; 0.5-4.0 min, gradient to 60% B; 4.0-5.0 min, 60% B; 5.0-6.0 min, gradient to 95% B; 6.0-10.0 min, 95% B—flow rate: 0.4 mL/min).

Mass spectrometric analysis was performed on a linear ion trap triple quadrupole mass spectrometer (4000 Q TRAP; AB SCIEX) equipped with a turbo spray ion source; mass spectrometer was operated in positive ion mode and quantification was achieved by multiple reaction monitoring (MRM) mode. Peak integration was performed using Analyst (version 1.6.2.). GraphPad Prism (version 8.1.2) was used for drawing graphs and for statistical analysis.

### Results

Plasma and tissue exposures of Compound 19 were determined at the three defined timepoints and are shown in FIG. 4. Consistent with the presence of a carboxylic acid moiety, plasma and tissue levels of Compound 19 showed limited lung partition (mean lung/plasma ratio of 0.4) and very low CNS (Central nervous system) exposure (mean brain/plasma ratio of 0.01).

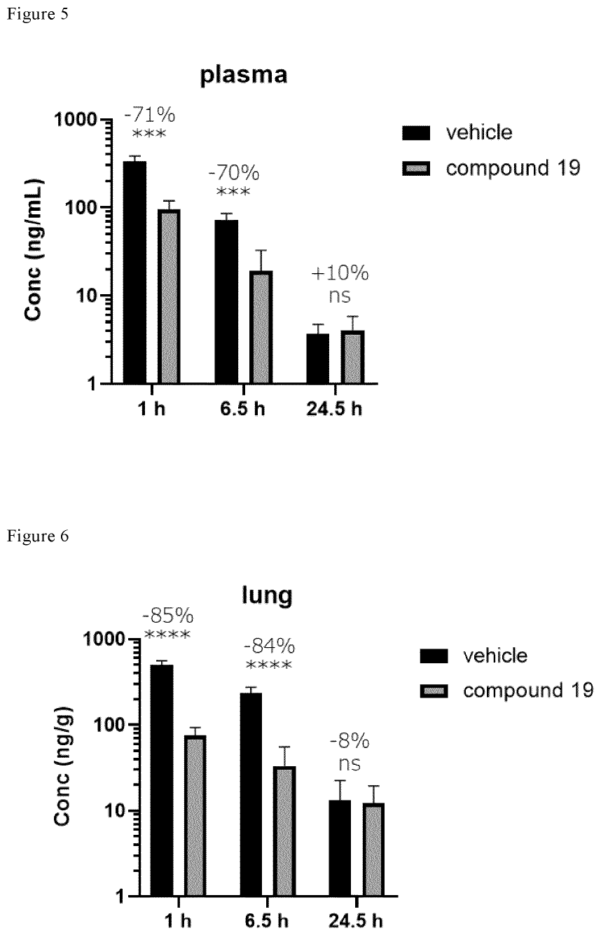

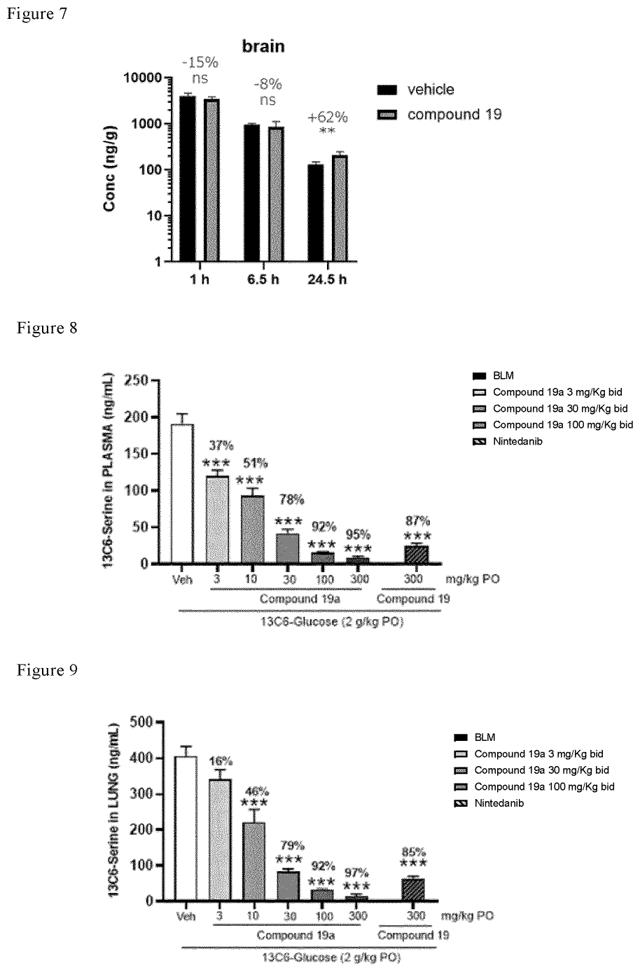

As shown in FIGS. 5 and 6, observed concentrations were able to produce significant inhibition of labelled serine in plasma and lung at 1 and 6.5 h (between 70 and 85%). As shown in FIG. 7, labelled serine levels in brain were significantly higher than plasma and lung ones, but no significant inhibition was observed at any timepoint, as expected based on the low concentrations observed for Compound 19 in the brain (unpaired t test at each timepoint, comparison between treated and vehicle).

### Example 4: Comparison of Compound 19 and Compound 19a in PK/PD Model

### Methods

After 0.5 h from administration of Compound 19a at dose of 3, 10, 30, 100 and 300 mg/kg po, Compound 19 at dose of 300 mg/kg po or vehicle po (0.5% Tween 80, 0.5% Methylcellulose in H2O), mice received ¹³C₆-glucose by oral gavage (2 g/kg, Merck Life Science Srl). At the established time-points (1 h after treatment with compounds or vehicle), animals were terminally anesthetized by intraperitoneal injection of thiopental (200 mg/kg/10 ml; MSD Animal Health Srl) and all tissues were harvested in less than 5 minutes to preserve the metabolic state and immediately frozen in liquid nitrogen. Blood was withdrawn by cardiac puncture and collected in heparin tubes. Plasma was obtained after centrifugation of blood at 10,000 g for 10 min.

### Results

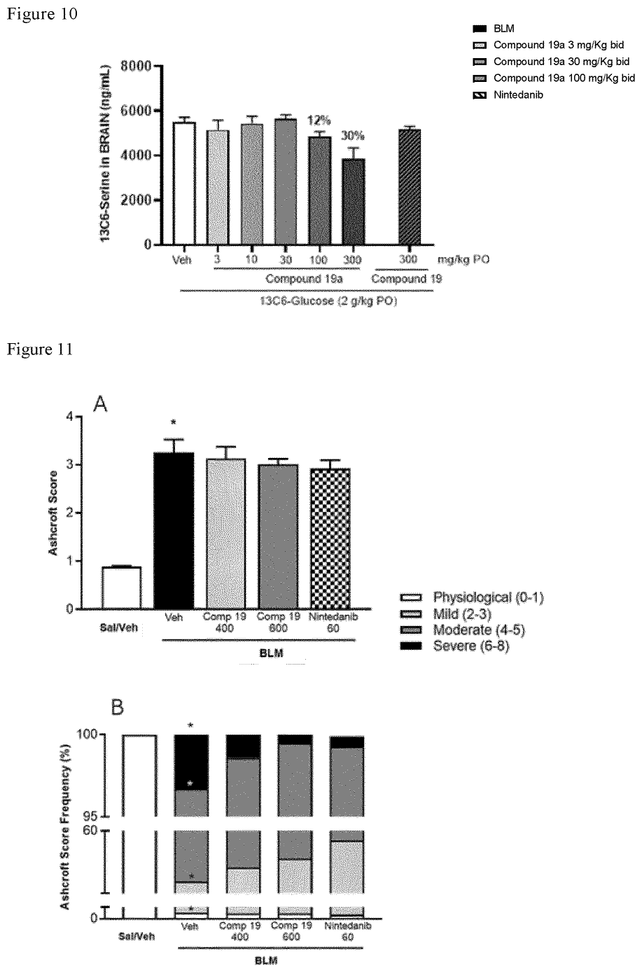

As shown in FIGS. 8 and 9 both Compounds 19 and 19a were able to lead to significant inhibition of labelled serine showing a dose-response curve in plasma and lung. Particularly, Compound 19a significantly reduced at all tested doses the concentration of labeled serine, producing a dose-dependent inhibition in plasma between 37% and 95% (FIG. 8) and in lung tissue between 16% and 97% (FIG. 9) with an ED80 estimated around 30 mg/kg in both tissues. Compound19 produced an inhibition in plasma and lung respectively by 87% (***p≤0.001) and 85% (***p≤0.001). Brain serine concentration was slightly decreased with the higher doses of Compound 19a (12% at 100 and 30% at 300 mg/kg), but without reaching the statistical significance similarly to the reference Compound 19 (FIG. 10).

### Example 5: Effect of the Compound 19 on the Development of Bleomycin-Induced Pulmonary Fibrosis in Male Mice

The effect of Compound 19 has accordingly been assessed in this murine model, in which fibrosis was induced by double oropharyngeal instillation of bleomycin. The C57BL/6J mouse has been chosen to evaluate the effects of the test compounds since it is more susceptible to bleomycin-induced fibrosis than other mouse strain. Compound 19 was administered under a therapeutic regimen, after the peak of the acute inflammatory phase of the lung injury response (at day 7 from first Bleomycin instillation), to be sure to better evaluate anti-fibrotic effect separately from anti-inflammatory effects.

### Methods

Male C57BL6/J mice (7-8 weeks old) were purchased from the Envigo RMS (San Pietro al Natisone (UD), Italy). All mice were maintained under pathogen-free conditions and were provided food and water ad libitum.

Lung injury was induced by double oropharyngeal instillation of Bleomycin hydrochloride (BLM, BAXTER) at day 0 and day 4 at the doses of 0.02 U/mouse in 50 μl of saline solution (0.9%). Control animals received double oropharyngeal instillation of 50 μl of saline solution.

Compound 19 was orally administered twice daily, starting on day 7 after the first oropharyngeal administration of bleomycin at the two doses of 400 and 600 mg/kg/day, 10 ml/kg, dissolved in 0.5% Tween 80, 0.5% Methylcellulose in H₂O and treatment lasted for two weeks.

A group of animals was treated with Nintedanib esylate salt, at the dose of 60 mg/kg/day, 10 ml/kg, once daily, to compare the efficacy of the test compounds with an FDA approved drug for the treatment of IPF patients. Control mice were given vehicle-only over the same time intervals. Weight of all animals has been assessed twice a week starting from day 0 until day 21.

On days 21, mice were anesthetized by intraperitoneal injection of thiopental (200 mg/kg/10 ml; pentothal sodium, MSD Animal Health Srl). Blood was withdrawn by cardiac puncture and collected in heparine tubes. Plasma was obtained after centrifugation of blood at 10,000 g for 10 min. Lungs were lavage with saline solution through the heart, formalin fixed and paraffin embedded, and sections were stained with Masson's Trichrome to evaluate the grade and severity of fibrosis and collagen deposition.

A score evaluation was made on the Masson's Trichrome stained slides, using a method based on Ashcroft scale (grade 0 to 8), as described by Ashcroft et al. (see Ashcroft T, Simpson J M, Timbrell V, J Cin Pathol 1988; 41(4):467-70) and modified by Hubner et al. (see Hubner R-H, Gitter W, Eddine El Mokhtari N, et al., Biotechniques. 2008; 44(4):507-517).

Masson's Trichrome stained slides were also scanned with NanoZoomer S60 (Hamamatsu Photonics K.K., Shizuoka, Japan) and acquired into the Visiopharm Integrator System (VIS; version 2017.2.4.3387) for a quantification of pulmonary fibrosis by automated analysis with the support of a VIS Analysis Protocol Package (APP). The present APP has been designed to identify and then quantify the fibrotic tissue, detecting accumulation of altered connective tissue, excess of collagen deposition and cellular density.

To quantify lung protein content and the levels of specific proteins as markers of fibrosis and collagen deposition, lungs from subgroups of animals were weighted, homogenized in Phosphate buffer solution (PBS) and proteins of interest were quantified by immunometric or colorimetric assays. Hydroxyproline content was determined using the Hydroxyproline Colorimetric Assay Kit (Sigma MAK008, USA) according to the manufacturer's protocol. WISP-1, CollagenI, pro-CollagenI and Elastin were determined by Elisa kits. Protein quantification was performed using DC Protein Assay (Bio-Rad Laboratories, Italy). Plasma serine levels were measured using the DL-Serine Assay Kit (Abcam, Cambridge, MA).

The experimental design used to test the effect of the Compound 19 on the development of bleomycin-induced pulmonary fibrosis in mice included 5 groups of 20 animals, as reported in Table 4.

TABLE 4
Experimental design for Compound 19

| Group | Treatment |
|---|---|
| 1 | Saline/vehicle n = 20 |
| 2 | BLM/Vehicle n = 20 |
| 3 | BLM/Compound 19 400 mg/Kg/day n = 20 |
| 4 | BLM/Compound 19 600 mg/Kg/day n = 20 |
| 5 | BLM/Nintedanib esylate 60 mg/Kg/day n = 20 |

In bleomycin-induced pulmonary fibrosis model in mice, body weight was reduced during the first 7 days after the first oropharyngeal instillation of bleomycin, then recovered slowly until 21 days.

Mice treated with Compound 19 or Nintedanib did not lose additional weight during the 2-weeks of treatment, compared to BLM/Vehicle treated group.

No significant differences in survival were observed between all groups of animals and only one animal died during treatment with Compound 19 at the highest tested dose against two animals died in the BLM/Vehicle group.

The lung/body weight of bleomycin-treated mice significantly increased compared to controls, as indication of increased inflammation, fluid accumulation, matrix deposition and finally fibrosis in the lungs.

Compound 19 showed a not significant tendency in reducing the lung/body weight (−31% at the highest dose) slightly superior to that induced by Nintedanib (−21%).

At histological level, animals treated with bleomycin developed, at day 21, expected lesions consistent with pulmonary fibrosis, with collagen deposition in lungs and partial obliteration of lung architecture.

Ashcroft score showed increased fibrosis in bleomycin treated mice relative to control mice, which was reduced by treatment with Compound 19 similarly to Nintedanib as shown in FIG. 11A. Furthermore, as reported in FIG. 11B, the Compound 19 specifically reduced the frequency of the most severe scores of fibrosis, showing a trend in increasing the mild ones.

The quantification of fibrotic tissue by automated analysis confirms a significant increase in Bleomycin-induced lung fibrosis in mice treated animals at day 21 that correlated with the Ashcroft score analysis.

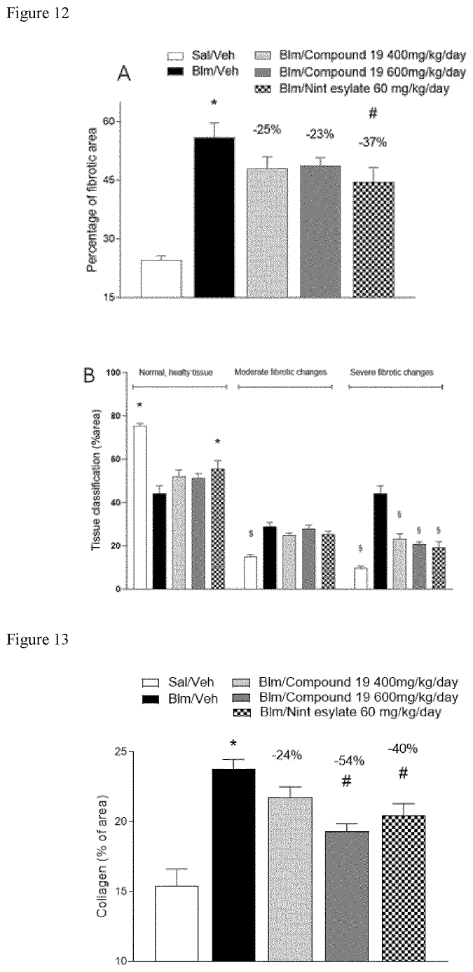

Compound 19 induced a ≅25% of reduction of tissue area occupied by fibrotic lesions and it induced a significant reduction of severity of fibrosis, as shown in FIG. 12.

Finally, the automatic collagen deposition quantification revealed a reduction of collagen content with all treatments that reached statistical significance with the highest dose of Compound 19, as shown in FIG. 13.

The Bleomycin induced a significant increase on lung levels of some proteins, markers of collagen accumulation (Hydroxyproline, HYP; Collagen I) and matrix deposition (WISP-1). The content of these proteins was significantly reduced following treatment with Compound 19 and the effect was even higher than that induced by Nintedanib, as shown in FIGS. 14A, 14B and 14C.

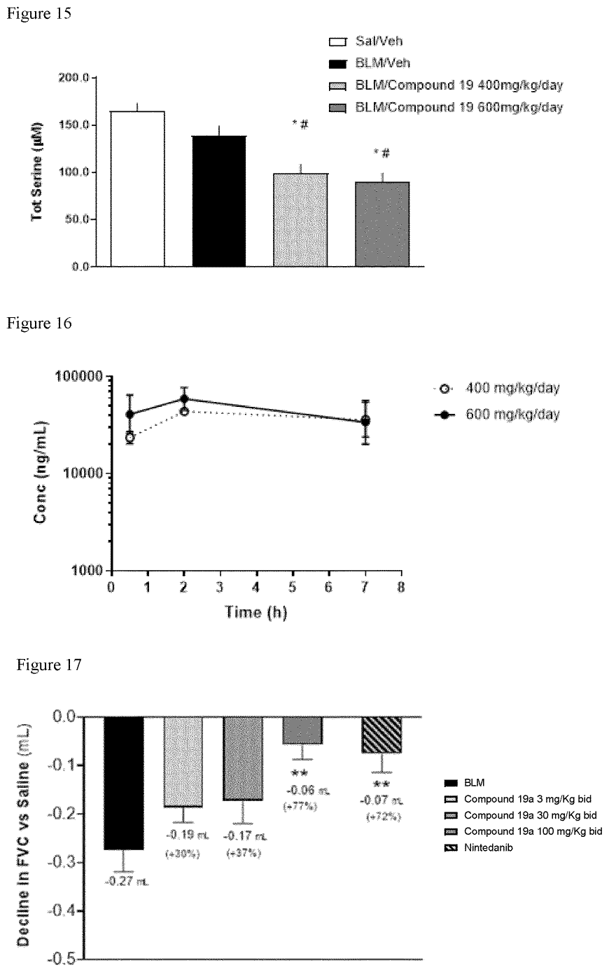

The inhibition of de novo serine synthesis induced by the PHGDH inhibition has been confirmed though the detection of the circulating serine levels in plasma of the Compound 19-treated animals. A significant reduction at both doses of the test compound 19 has been observed as shown in FIG. 15.

The Bioanalysis of Compound 19 in plasma was carried out under the same experimental conditions as reported in Example 3.

Plasma exposure of Compound 19 was determined following the last oral dose at three timepoints (0.5 h, 2 h and 7 h). PK data are shown in FIG. 16 and indicative of good oral exposure.

### Example 6: Effect of the Compound 19a on the Development of Bleomycin-Induced Pulmonary Fibrosis in Male Mice

The effect of Compound 19a was evaluated in a murine model (C57BL/6J mouse strain) in which fibrosis was induced by a double oropharyngeal (OA) instillation of bleomycin. The Compound 19a was administered under therapeutic regimen for 14 days, starting the treatments at day 7 from the first instillation of Bleomycin OA up to the day 21, considered the endpoint for this model.

### Methods

Male C57BL6/J mice (7-8 weeks old) were purchased from the Envigo RMS (San Pietro al Natisone (UD), Italy). All mice were maintained under pathogen-free conditions and had free access to food and water.

Lung injury was induced by double OA instillation of bleomycin hydrochloride (BLM, BAXTER, Deerfield, IL, USA) at day 0 and day 4 at the dose of 0.03 U/mouse in 50 L of saline solution (NaCl 0.9%). Control animals received double OA instillation of 50 L of saline solution.

The Compound 19a was orally administered twice daily at the three doses of 3, 30 and 100 mg/Kg 10 mL/Kg, using as vehicle for dissolution the 0.5% Tween 80, 0.5% Methylcellulose in H₂O.

An experimental group was treated with Nintedanib esylate salt at the dose of 50 mg/Kg 10 ml/kg, twice daily, to compare the efficacy of the test compound with the FDA-approved drug in IPF treating patients. Control mice were given vehicle-only over the same time intervals.

On day 21, the respiratory system mechanics were measured using the FlexiVent System (SCIREQ Inc., Montreal Qc, Canada) (Vanoirbeek et al., 2010).

Briefly, mice were anesthetized by intraperitoneal injection of ketamine (Anesketin®, Dechra Inc., Handelsweg, NL) and xylazine (Xilagesic®, Calier Inc., Barcelona, ES) solution (100 mg/Kg and 10 mg/Kg, respectively). Once anesthesia was achieved, to analyze forced vital capacity (FVC), anesthetized mice were tracheotomized using an 18-gauge metal cannula, connected via an endotracheal cannula to the FlexiVent and ventilated at a respiratory rate of 150 breaths/min and tidal volume of 10 mL/Kg against a positive end-expiratory pressure of 3 cm H₂O to achieve a mean lung volume close to that during spontaneous breathing. To prevent spontaneous breathing mice also received pancuronium bromide at 1 mg/Kg intraperitoneally. FlexiVent software version 8.1 was used to perform the perturbations.

Immediately after the in-life evaluation, plasma and bronchoalveolar lavage fluid (BALF) samples were collected.

First, blood was collected by cardiac puncture, and plasma was obtained after centrifugation of blood at 2000×g and 4° C. for 10 minutes.

Subsequently, the lungs were gently rinsed three times using a tracheal cannula with 0.6 mL of clear BAL solution. The obtained BALF was centrifuged at 1000×g and 4° C. for 10 minutes. The cell-free supernatant was used for biomarkers quantitative determination.

The following biomarkers were determined in BALF and plasma samples by commercially available ELISA kits: Collagen I (evaluated in BALF), an index of collagen accumulation; matrix metallopeptidase 7 (MMP-7, evaluated in BALF), indicator of aberrant fibrogenesis and ECM remodeling and surfactant protein D (SP-D, evaluated in PLASMA), marker of alveolar epithelial cell damage and dysfunction.

Lastly, the entire lungs were resected, fixed with formalin and embedded in paraffin. Sections were stained with Masson's trichrome to assess the grade and severity of fibrosis and collagen deposition.

The score evaluation was made on the Masson's Trichrome stained slides, using the Ashcroft scale (grade 0 to 8) (see Hübner R-H, Gitter W, Eddine El Mokhtari N, et al., Biotechniques. 2008; 44(4):507-517).

Slides were also scanned with NanoZoomer S60 (Hamamatsu Photonics K.K., Shizuoka, Japan) and acquired into the Visiopharm Integrator System (VIS; version 2017.2.4.3387) for pulmonary fibrosis quantification by automated analysis with the support of a VIS Analysis Protocol Package (APP). The present APP has been designed to identify and then quantify the fibrotic tissue, detecting accumulation of altered connective tissue, excess of collagen deposition and cellular density.

The experimental design used to test the effect of the Compound 19a on the development of bleomycin-induced pulmonary fibrosis in mice included 6 groups (N=7-14), as reported in Table 5:

TABLE 5
Experimental design for Compound 19a

| Group | Treatment |
|---|---|
| 1 | Saline/vehicle N = 7 |
| 2 | BLM/Vehicle N = 14 |
| 3 | BLM/Compound 19a 3 mg/Kg/bid N = 14 |
| 4 | BLM/Compound 19a 30 mg/Kg/bid N = 14 |
| 5 | BLM/Compound19a 100 mg/Kg/bid N = 14 |
| 6 | BLM/Nintedanib esylate salt 50 mg/Kg/bid N = 13 |

### Results

A consistent decline of 0.27 mL in FVC was induced by bleomycin administration compared to saline group. The Compound 19a significantly (**p≤0.01) improved by 77% the FVC decline at the dose of 100 mg/kg similarly to Nintedanib when compared to BLM/vehicle group (FIG. 17).

The bleomycin instillation induced a significant increase in lung levels of Collagen I and MMP-7 evaluated in BALF (FIGS. 18A and 18B). The content of these biomarkers was significantly reduced in a dose dependent manner by Compound 19a, getting the maximum effect at dose of 100 mg/Kg comparably with Nintedanib for Collagen I (Compound 19a reduction of 52%, *p≤0.05 vs Nintedanib reduction of 56%, *p≤0.05) but superior for MMP-7 (Compound 19a reduction of >100% ***p≤0.001 vs Nintedanib reduction of 86%, *p≤0.05).

In plasma samples a significant (**p≤0.01) increase in SP-D levels has been reported in injured animals. The animal groups treated with Compound 19a showed a dose-dependent reduction of SP-D levels at the dose of 100 mg/Kg reported a significant decrease (reduction of 74%, *p≤0.05) in SP-D levels slightly inferior to Nintedanib (reduction of 87%, **p≤0.01) (FIG. 18C).

At histological level, animals treated with bleomycin developed lesions consistent with pulmonary fibrosis, with collagen deposition in lungs and partial alteration of lung architecture.

Ashcroft score showed in the BLM/vehicle group, compared with control mice, a significant (***p≤0.001) increase in fibrosis, which was markedly reduced by Compound 19a treatment of 56% (*p≤0.05) (FIG. 19A).

The fibrotic tissue quantification by automated analysis confirms a significant increase in lung fibrosis represented by severe and moderate tissues in bleomycin-induced mice. The Compound 19a was able to reduce in a dose dependent manner the fibrotic tissue getting an effect comparable to Nintedanib at the dose of 100 mg/Kg (FIG. 19 B).

### Comparative Example 7

### Effect of NCT-503 on the Development of Bleomycin-Induced Pulmonary Fibrosis in Male Mice

The allosteric inhibitor NCT-503 has accordingly been assessed in the same murine model of Compound 19. Comparative Example 7 was carried out under the same experimental conditions of Example 5.

The experimental design used to test the effect of the allosteric compound NCT-503 on the development of bleomycin-induced pulmonary fibrosis in mice included 5 groups of 20 animals, as reported in Table 6:

TABLE 6
The experimental design for NCT-503

| Group | Treatment |
|---|---|
| 1 | Saline/vehicle n = 20 |
| 2 | BLM/Vehicle n = 20 |
| 3 | BLM/NCT-503 100 mg/Kg/day n = 20 |
| 4 | BLM/NCT-503 300 mg/Kg/day n = 20 |
| 5 | BLM/Nintedanib esylate 60 mg/Kg/day n = 20 |

The NCT-503 was orally administered with the same protocol of treatment, bid daily, at the initial doses of 200 and 600 mg/kg/day, 10 ml/kg, dissolved in 1% Tween 80, 0.5% Methylcellulose in H₂O.

Animals did not tolerate treatment with the highest dose of NCT-503 and a severe decline of the Bleomycin-induced weight loss (~20% compared to Day 0) was observed. The treatment was stopped at day 10, after 4 days of treatment with NCT-503 600 mg/kg/day. After two days wash-out, the treatment re-started with a once daily regimen at the final dose of 300 mg/kg/day.

Animals from this group recovered weight until the end of experiment, however some animals died during the experiment and the survival decreased to 40%. This experimental group was not included in the final analysis.

Although animals treated with NCT-503 at the dose of 200 mg/kg/day did not show a significant worsening of the body weight, the chosen treatment was with a once daily regimen until the end of experiment. The final dose was of 100 mg/kg/day.

Bleomycin administration led to a significant increase in lung weight when compared to controls; the treatment with NCT-503 and Nintedanib do not induce reduction of lung/body weight.

Mice treated with NCT-503 did not result in diminished Bleomycin-induced lung fibrosis, measured by the Ashcroft score analysis, when compared to vehicle-treated mice, as shown in FIG. 20A. Moreover, NCT-503 showed a not significant trend in reducing fibrosis severity, FIG. 20B.

The levels of some markers of collagen accumulation (pro-CollagenI) and matrix deposition (WISP-1), were not affected by the treatment with NCT-503, as shown in FIGS. 21A and 21B.

NCT-503 did not induce inhibition of de novo serine synthesis as measured by circulating serine levels of the NCT-503-treated animals, as shown in FIG. 22.

Plasma and tissue exposures of NCT-503 were determined as described in Example 2. The plasma and brain concentrations of NCT-503 were determined by a liquid chromatography-tandem mass spectrometry (LC/MS/MS) assay. Plasma or tissue homogenate samples (tissues were homogenized with a water/acetonitrile 50/50 mixture) were extracted with 3 volumes of acetonitrile. Supernanatant was injected into HPLC system (Agilent 1260 Quaternary Pump equipped with an Agilent 1200 High Performance Autosampler SL; Agilent), using a Kinetex EVO C18 analytical column (100 A, 50×2.1 mm, 2.6 μm; Phenomenex). The mobile phases consisted of 0.1% formic acid in water (solvent A) and 0.1% formic acid in acetonitrile (solvent B); chromatographic separation was performed with gradient elution method (0.0-0.5 min, 5% B; 0.5-4.0 min, gradient to 90% B; 4.0-5.5 min, 90% B; 5.5-7.0 min, gradient to 5% B; 7.0-11.0 min, 5% B—flow rate: 0.3 mL/min).

Mass spectrometric analysis was performed on a linear ion trap triple quadrupole mass spectrometer (4000 Q TRAP; AB SCIEX) equipped with a turbo spray ion source; mass spectrometer was operated in positive ion mode and quantification was achieved by multiple reaction monitoring (MRM) mode. Peak integration was performed using Analyst (version 1.6.2.). GraphPad Prism (version 8.1.2) was used for graphs.

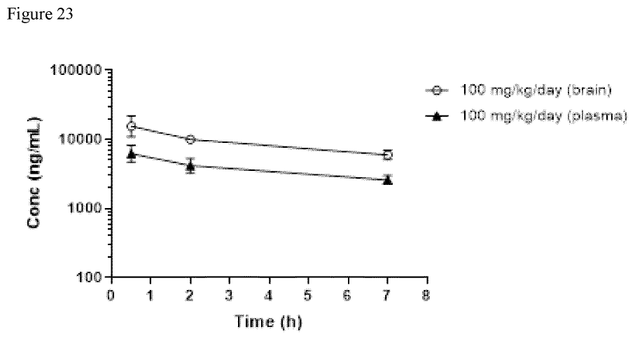

Plasma and brain exposure of NCT-503 was determined following the last oral dose at three timepoints (0.5 h, 2 h and 7 h). PK data are shown in FIG. 23.

The NCT-503 showed very high CNS (Central nervous system) exposure if compared to Compound 19 or Compound 19a, as demonstrated by the main brain/plasma ratio of 2.4.

1: A method of treating fibrosis, comprising administering a compound of Formula (I), or a pharmaceutically acceptable salt thereof, to a subject in need thereof, 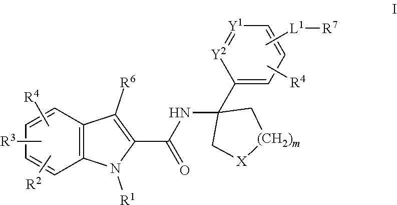
wherein
R¹ is hydrogen or C₁₋₄ alkyl;
each of R² and R³ is independently halogen, —OR, —CN, C₁₋₆ aliphatic optionally substituted with 1, 2, or 3 halogens, or -L-R⁸; or R² and R³ are optionally taken together with the carbon atoms to which they are attached and any intervening atoms to form a 5-8 membered partially unsaturated ring having 0-2 heteroatoms independently selected from nitrogen, oxygen, or sulfur;
each L is independently a C₁₋₆ bivalent straight or branched hydrocarbon chain wherein 1-4 methylene units of the chain are independently and optionally replaced with —O—, —C(O)—, —C(O)O—, —OC(O)—, —OC(O)N(R)—, —(R)NC(O)O—, —C(O)N(R)—, —(R)NC(O)—, —N(R)—, —N(R)C(O)N(R)—, —S—, —SO—, or —SO₂—;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
R⁸ is hydrogen, C₁₋₆ aliphatic, or an optionally substituted 4-8 membered saturated or partially unsaturated monocyclic heterocyclic ring having 1-3 heteroatoms independently selected from nitrogen, oxygen, or sulfur;
R⁴ is hydrogen, halogen, —OR⁵, —CN, C₁₋₆ aliphatic optionally substituted with 1, 2, or 3 halogens, or -L-R⁸
R⁵ is hydrogen, —(CH₂)~-phenyl, —(CH₂)ₙ-Cy′, or C₁₋₆ alkyl optionally substituted with 1, 2, or 3 halogens;
each -Cy′- is a 4-8 membered saturated or partially unsaturated monocyclic heterocyclic ring having 1-2 heteroatoms independently selected from nitrogen, oxygen, or sulfur,
R⁶ is hydrogen or C₁₋₄ alkyl;
R⁷ is hydrogen, —CO₂R, optionally substituted C₁₋₆ aliphatic, -Cy-, or a bivalent 3-7 membered ring;
L¹ is a covalent bond or a C₁₋₈ bivalent straight or branched hydrocarbon chain wherein 1-5 methylene units of the chain are independently and optionally replaced with —O—, —C(O)—, —C(O)O—, —OC(O)—, —N(R)—, —C(O)N(R)—, —(R)NC(O), —OC(O)N(R), —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R), or -Cy-;
each -Cy- is independently a bivalent 6-membered arylene ring containing 0-2 nitrogen atoms, or a bivalent 5-membered heteroarylene ring with 1-4 heteroatoms independently selected from nitrogen, oxygen, or sulfur, or a bivalent partially unsaturated 8-10 membered bicyclic heterocyclene ring with 1-4 heteroatoms independently selected from nitrogen, oxygen, or sulfur, wherein -Cy- is optionally substituted with 1 or 2 substituents independently selected from C₁₋₄ alkyl or —OR;
X is O, S, or —N(R¹⁰)—;
R¹⁰ is C₁₋₆ aliphatic optionally substituted with 1, 2, or 3 halogens, —C(O)CH₃, or —SO₂—N(R¹)(R¹¹);
R¹¹ is —C(O)CH₃, —C(O)NHR¹, or pyrazinyl;
n is independently 0, 1, 2, 3, 4, or 5;
m is independently 0, 1, or 2; and
each of Y¹ and Y² is independently ═N— or ═C(R⁴)—.

2: The method according to claim 1, wherein R¹ is methyl.

3: The cm method according to claim 1 wherein R² is selected from the group consisting of F, Cl, —CF₃, —OCF₃, —OCHF₂, —OCH₂Ph, —OCH₃, —CN, —CH₃,

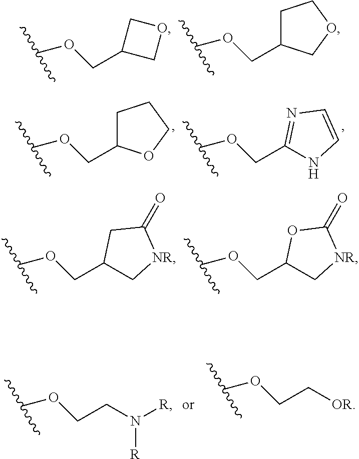

4: The method according to claim 1 wherein R² is selected from the group consisting of F, Cl, —OCH₃ and —CH₃.

5: The method according to claim 1 wherein R³ is selected from the group consisting of F, Cl, —OCH₃ and —CH₃.

6: The method according to claim 1 wherein R⁴ is hydrogen.

7: The method according to claim 1 wherein R⁶ is hydrogen or methyl.

8: The method according to claim 1 wherein L¹ is selected from the group consisting of SO₂NH—,

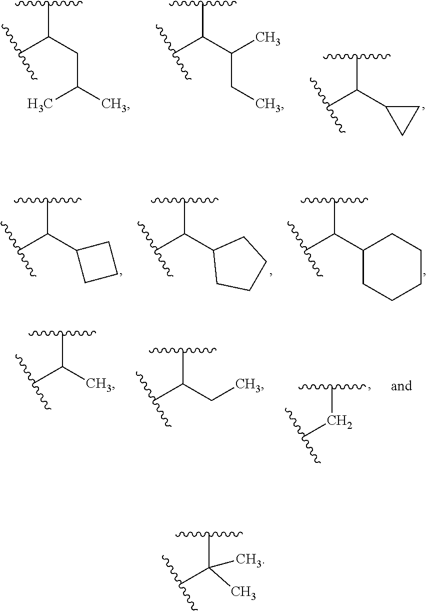

9: The method according to claim 1 wherein R⁷ is selected from the group consisting of hydrogen, methyl, ethyl, cyclopropyl, cyclobutyl, tetrazolyl, and —CO₂H.

10: The method according to claim 1, wherein:
X is O; and
Y¹ and Y² are CH.

11: The method according to claim 1, wherein said compound is selected from Formulae II-a, II-b, II-c, II-d, or II-e: 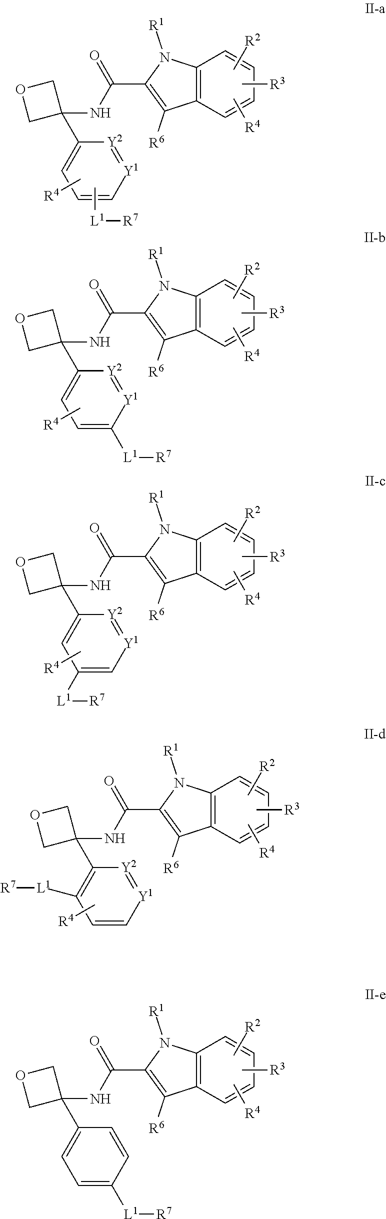
wherein each of R¹, R², R⁴, R⁶, R⁷, L¹, Y¹ and Y² are as defined in claim 1.

12: The method according to claim 1, wherein said compound is selected from Formulae III-a, III-b III-c III-d III-e, III-f, III-g, III-h, or III-i: 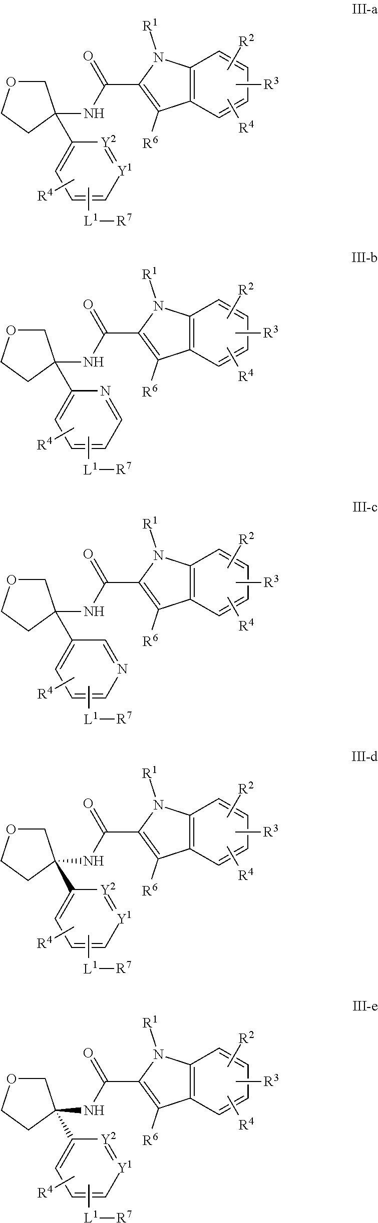 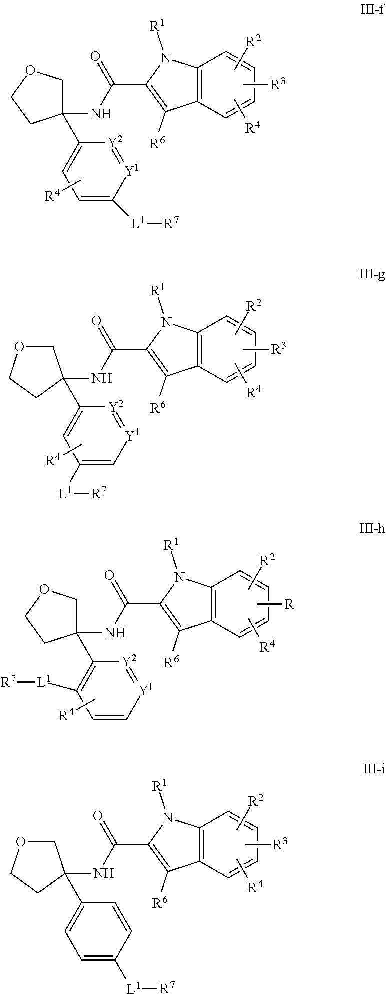
wherein each of R¹, R², R⁴, R⁶, R⁷, L¹, Y¹ and Y² are as defined in claim 1.

13: The method according to claim 1, wherein the compound is at least one selected from the group consisting of:
N-(3-(4-(N-Acetylsulfamoyl)phenyl)oxetan-3-yl)-4,5-dichloro-1-methyl-1H-indole-2-carboxamide;
(±)-3-{4-[3-(4,5-dichloro-1-methyl-1H-indole-2-amido)oxetan-3-yl]phenyl}oxolane-3-carboxylic acid;
(±)-3-cyclopropyl-2-{4-[3-(4,5-dichloro-1-methyl-1H-indole-2-amido)oxetan-3-yl]phenyl}propanoic acid;
(±)-3-cyclopropyl-2-{4-[3-(4,5-dichloro-6-methoxy-1-methyl-1H-indole-2-amido)oxetan-3-yl]phenyl}propanoic acid;
2R)-2-[4-[3-[(4-chloro-5-methoxy-1-methyl-indole-2-carbonyl)amino]oxetan-3-yl]phenyl]-2-cyclopentyl-acetic acid;
(2S)-2-[4-[3-[(4-chloro-5-methoxy-1-methyl-indole-2-carbonyl)amino]oxetan-3-yl]phenyl]-2-cyclopentyl-acetic acid;
(±)-2-cyclobutyl-2-{4-[3-(4,5-dichloro-6-methoxy-1-methyl-1H-indole-2-amido)oxetan-3-yl]phenyl}acetic acid;
(±)-2-cyclobutyl-2-{4-[3-(4,5-dichloro-1-methyl-1H-indole-2-amido)oxetan-3-yl]phenyl}acetic acid;
(±)-2-(4-(3-(4,5-Dichloro-1-methyl-1H-indole-2-carboxamido)oxetan-3-yl)phenyl)-4-methylpentanoic acid;
(±)-2-{4-[3-(4,5-dichloro-6-methoxy-1-methyl-1H-indole-2-amido)oxetan-3-yl]phenyl}-4-methylpentanoic acid;
2-{4-[3-(4-chloro-5-methoxy-1-methyl-1H-indole-2-amido)oxetan-3-yl]phenyl}-5-hydroxypentanoic acid;
(±)-2-cyclobutyl-2-{4-[3-(4,5-dichloro-1-methyl-1H-indole-2-amido)oxolan-3-yl]phenyl}acetic acid;
(±)-2-{4-[3-(4-chloro-5-methoxy-1-methyl-1H-indole-2-amido)oxetan-3-yl]phenyl}-2-cyclobutylacetic acid;
(±)-2-{4-[3-(4,5-dichloro-6-methoxy-1-methyl-1H-indole-2-amido)oxetan-3-yl]phenyl}butanoic acid;
(±)-2-Cyclopropyl-2-(4-(3-(4,5-dichloro-1-methyl-1H-indole-2-carbox-amido)tetrahydrofuran-3-yl)phenyl)acetic acid;
(±)-2-cyclopentyl-2-{4-[3-(4,5-dichloro-1-methyl-1H-indole-2-amido)oxolan-3-yl]phenyl}acetic acid;
(±)-2-{4-[3-(4,5-dichloro-6-methoxy-1-methyl-1H-indole-2-amido)oxolan-3-yl]phenyl}pentanoic acid;
(±)-2-{4-[3-(4,5-dichloro-1-methyl-1H-indole-2-amido)oxolan-3-yl]phenyl}pentanoic acid;
(±)-2-(4-(3-(4,5-Dichloro-1-methyl-1H-indole-2-carboxamido)tetrahydro-furan-3-yl) phenyl)-3-methylbutanoic acid;
Diasteroisomer 1 of (±)-2-(4-(3-(4,5-Dichloro-1-methyl-1H-indole-2-carboxamido)tetrahydro-furan-3-yl) phenyl)-3-methylbutanoic acid;
Diasteroisomer 2 of (±)-2-(4-(3-(4,5-Dichloro-1-methyl-1H-indole-2-carboxamido)tetrahydro-furan-3-yl) phenyl)-3-methylbutanoic acid;
Diasteroisomer 3 of (±)-2-(4-(3-(4,5-Dichloro-1-methyl-1H-indole-2-carboxamido)tetrahydro-furan-3-yl) phenyl)-3-methylbutanoic acid;
Diasteroisomer 4 of (±)-2-(4-(3-(4,5-Dichloro-1-methyl-1H-indole-2-carboxamido)tetrahydro-furan-3-yl) phenyl)-3-methylbutanoic acid;
(±)-2-{4-[3-(4,5-dichloro-6-methoxy-1-methyl-1H-indole-2-amido)oxolan-3-yl]phenyl}-3-methylbutanoic acid;
(±)-2-[4-[3-[(4-Chloro-5-methoxy-1-methyl-indole-2-carbonyl)amino]-oxetan-3-yl]phenyl]-3-methyl-butanoic acid;
(±)-2-cyclopentyl-2-{4-[3-(4,5-dichloro-6-methoxy-1-methyl-1H-indole-2-amido)oxetan-3-yl]phenyl}acetic acid;
(±)-2-{4-[3-(4,5-dichloro-6-methoxy-1-methyl-1H-indole-2-amido)oxolan-3-yl]phenyl}butanoic acid;
(±)-2-{4-[3-(4,5-dichloro-1-methyl-1H-indole-2-amido)oxetan-3-yl]phenyl}pentanoic acid;
(±)-2-Cyclopentyl-2-[4-[3-[(4,5-dichloro-1-methyl-indole-2-carbonyl) amino]oxetan-3-yl]phenyl]acetic acid;
(±)-2-[4-[3-[(4-chloro-5-methoxy-1-methyl-indole-2-carbonyl)amino]-oxetan-3-yl]phenyl]-2-cyclopentyl-acetic acid;
2-{4-[3-(4-chloro-5-methoxy-1-methyl-1H-indole-2-amido)oxetan-3-yl]phenyl}-2-methylpropanoic acid;
1-{4-[3-(4,5-dichloro-1-methyl-1H-indole-2-amido)oxetan-3-yl]phenyl}cyclopropane-1-carboxylic acid;
(±)-2-[4-[3-[(4,5-Dichloro-6-methoxy-1-methyl-indole-2-carbonyl)amino]oxetan-3-yl]phenyl]-3-methyl-butanoic acid;
(±)-2-[4-[3-[(4,5-Dichloro-1-methyl-indole-2-carbonyl)amino]oxetan-3-yl]phenyl]-3-methyl-butanoic acid;
(±)-2-{4-[3-(4-chloro-5-methoxy-1-methyl-1H-indole-2-amido)oxetan-3-yl]phenyl}pentanoic acid;
2-{3-chloro-4-[3-(4-chloro-5-methoxy-1-methyl-1H-indole-2-amido)oxetan-3-yl]phenyl}acetic acid;
2-{3-chloro-4-[3-(4,5-dichloro-1-methyl-1H-indole-2-amido)oxetan-3-yl]phenyl}propanoic acid;
(R)-2-[4-[3-[(4,5-dichloro-1-methyl-indole-2-carbonyl)amino]oxetan-3-yl]phenyl]propanoic acid;
(S)-2-[4-[3-[(4,5-dichloro-1-methyl-indole-2-carbonyl)amino]oxetan-3-yl]phenyl]propanoic acid;
2-(4-(3-(4,5-Dichloro-1-methyl-1H-indole-2-carboxamido)oxetan-3-yl)Phenyl)-2-methylpropanoic acid;
2-{4-[3-(4,5-dichloro-1-methyl-1H-indole-2-amido)oxetan-3-yl]-3-fluorophenyl}-2-methylpropanoic acid;
(±)-2-{4-[3-(4,5-dichloro-6-methoxy-1-methyl-1H-indole-2-amido)oxetan-3-yl]phenyl}pentanoic acid;
(±)-2-{4-[3-(4-chloro-5-methoxy-1-methyl-1H-indole-2-amido)oxolan-3-yl]-3-fluorophenyl}-2-methylpropanoic acid;
(±)-2-{4-[3-(4-chloro-5-methoxy-1-methyl-1H-indole-2-amido)oxolan-3-yl]-3-fluorophenyl}propanoic acid;
5-Dichloro-1-methyl-N-[3-[4-(1H-tetrazol-5-ylmethyl)phenyl]oxetan-3-yl]indole-2-carboxamide;
(±)-2-(4-(3-(4,5-dichloro -1-methyl-1H-indole-2-carboxamido)oxetan-3-yl)phenyl)butanoic acid;
(±)-2-(4-(3-(4-chloro-5-methoxy-1-methyl-1H-indole-2-carboxamido)oxetan-3-yl) phenyl)butanoic acid;
3-chloro-4-[3-(4-chloro-5-fluoro-6-methoxy-1-methyl-1H-indole-2-amido)oxetan-3-yl]benzoic acid;
4-[3-(4-chloro-6-methoxy-1,5-dimethyl-1H-indole-2-amido)oxetan-3-yl]-3-methylbenzoic acid;
3-chloro-4-[3-(4-chloro-6-methoxy-1,5-dimethyl-1H-indole-2-amido)oxetan-3-yl]benzoic acid;
4-[3-(4-chloro-5-fluoro-6-methoxy-1-methyl-1H-indole-2-amido)oxetan-3-yl]-3-methylbenzoic acid;
2-{4-[3-(4,5-dichloro-6-methoxy-1-methyl-1H-indole-2-amido)oxetan-3-yl]phenyl}propanoic acid;
(±)-2-{4-[3-(4-chloro-5-methoxy-1-methyl-1H-indole-2-amido)oxetan-3-yl]-3-fluorophenyl}propanoic acid;
4-[3-(4-chloro-6-methoxy-1,5-dimethyl-1H-indole-2-amido)oxetan-3-yl]-3-fluorobenzoic acid;
4-[3-(4,5-dichloro-6-methoxy-1-methyl-1H-indole-2-amido)oxetan-3-yl]-3-methylbenzoic acid;
(±)—N-(3-(4-(1-(1H-tetrazol-5-yl)ethyl)phenyl)tetrahydrofuran-3-yl)-4,5-dichloro-1-methyl-1H-indole-2-carboxamide;
(±)-2-{4-[3-(4,5-dichloro-6-methoxy-1-methyl-1H-indole-2-amido)oxolan-3-yl]-3-fluorophenyl}-2-methylpropanoic acid;
(±)-2-{4-[3-(4,5-dichloro-1-methyl-1H-indole-2-amido)oxolan-3-yl]-3-fluorophenyl}-2-methylpropanoic acid;
(±)-2-{4-[3-(4,5-dichloro-6-methoxy-1-methyl-1H-indole-2-amido)oxolan-3-yl]-3-fluorophenyl}propanoic acid;
4-[3-(4-chloro-5-fluoro-6-methoxy-1-methyl-1H-indole-2-amido)oxetan-3-yl]-3-fluorobenzoic acid;
(±)-2-{4-[3-(4,5-dichloro-1-methyl-1H-indole-2-amido)oxolan-3-yl]-3-fluorophenyl}propanoic acid;
(±)-1-{4-[3-(4-chloro-5-methoxy-1-methyl-1H-indole-2-amido)oxolan-3-yl]phenyl}cyclopropane-1-carboxylic acid;
(±)-1-{4-[3-(4,5-dichloro-1-methyl-1H-indole-2-amido)oxolan-3-yl]phenyl}cyclopropane-1-carboxylic acid;
4-[3-(4-chloro-5-methoxy-1-methyl-1H-indole-2-amido)oxetan-3-yl]-3-methylbenzoic acid;
2-{4-[3-(4-chloro-5-fluoro-6-methoxy-1-methyl-1H-indole-2-amido)oxetan-3-yl]-3-fluorophenyl}acetic acid;
2-{4-[3-(4-chloro-6-methoxy-1,5-dimethyl-1H-indole-2-amido)oxetan-3-yl]-3-fluorophenyl}acetic acid;
2-{4-[3-(4-chloro-1,5-dimethyl-1H-indole-2-amido)oxetan-3-yl]-3-fluorophenyl}acetic acid;
(±)-2-{4-[3-(4-chloro-5-methoxy-1-methyl-1H-indole-2-amido)oxolan-3-yl]phenyl}propanoic acid;
3-chloro-4-[3-(4-chloro-5-methoxy-1-methyl-1H-indole-2-amido)oxetan-3-yl]benzoic acid;
3-chloro-4-[3-(4,5-dichloro-6-methoxy-1-methyl-1H-indole-2-amido)oxetan-3-yl]benzoic acid;
(±)-2-{4-[3-(4-chloro-5-methoxy-1-methyl-1H-indole-2-amido)oxetan-3-yl]phenyl}propanoic acid;
(±)-2-{4-[3-(4,5-dichloro-1-methyl-1H-indole-2-amido)oxetan-3-yl]phenyl}propanoic acid;
2-{4-[3-(4,5-dichloro-1-methyl-1H-indole-2-amido)oxetan-3-yl]-3-methoxyphenyl}acetic acid;
2-{4-[3-(4,5-dichloro-1-methyl-1H-indole-2-amido)oxetan-3-yl]-3-fluorophenyl}propanoic acid;
(±)-2-[3-Cyano-4-[3-[(4,5-dichloro-1-methyl-indole-2-carbonyl)amino]tetrahydro-furan-3-yl]phenyl]acetic acid;
(±)-2-{4-[3-(4,5-dichloro-1-methyl-1H-indole-2-amido)oxolan-3-yl]phenyl}butanoic acid;
(±)-5-[3-(4,5-dichloro-1-methyl-1H-indole-2-amido)oxolan-3-yl]pyridine-2-carboxylic acid;
(±)-6-[3-(4,5-dichloro-1-methyl-1H-indole-2-amido)oxolan-3-yl]pyridine-3-carboxylic acid;
4-[3-(4-chloro-5-methoxy-1-methyl-1H-indole-2-amido)oxetan-3-yl]-3-fluorobenzoic acid;
4-[3-(4,5-dichloro-6-methoxy-1-methyl-1H-indole-2-amido)oxetan-3-yl]-3-fluorobenzoic acid;
(±)-2-{4-[3-(4-chloro-5-methoxy-1-methyl-1H-indole-2-amido)oxolan-3-yl]phenyl}-2-methylpropanoic acid;
(±)-2-{4-[3-(4-chloro-5-methoxy-1-methyl-1H-indole-2-amido)oxolan-3-yl]phenyl}acetic acid;
4-[3-(4,5-dichloro-1-methyl-1H-indole-2-amido)oxetan-3-yl]-3-fluorobenzoic acid;
(±)-4-{3-[4,5-dichloro-1-methyl-6-(oxetan-3-ylmethoxy)-1H-indole-2-amido]oxolan-3-yl}benzoic acid;
(±)-4-(3-(6-((1H-imidazol-2-yl)methoxy)-4,5-dichloro-1-methyl-1H-indole-2-carboxamido) tetrahydrofuran-3-yl)benzoic acid;
2-{4-[3-(4,5-dichloro-1-methyl-1H-indole-2-amido)oxetan-3-yl]-3-methylphenyl}acetic acid;
4-[3-(4,5-dichloro-1-methyl-1H-indole-2-amido)oxetan-3-yl]-3-methylbenzoic acid;
2-{3-chloro-4-[3-(4,5-dichloro-1-methyl-1H-indole-2-amido)oxetan-3-yl]phenyl}acetic acid;
3-chloro-4-[3-(4,5-dichloro-1-methyl-1H-indole-2-amido)oxetan-3-yl]benzoic acid;
2-{4-[3-(4,5-dichloro-6-methoxy-1-methyl-1H-indole-2-amido)oxetan-3-yl]-3-fluorophenyl}acetic acid;
(±)-2-{4-[3-(4,5-dichloro-1-methyl-1H-indole-2-amido)oxolan-3-yl]phenyl}-2-methylpropanoic acid;
(±)-4-[3-[(4,5-Dichloro-1-methyl-indole-2-carbonyl)amino]tetrahydro-furan-3-yl]-2-ethyl- benzoic acid;
(±)-4-(3-(6-(3-Amino-2-hydroxypropoxy)-4,5-dichloro-1-methyl-1H-indole-2-carboxamido) tetrahydrofuran-3-yl)benzoic acid;
2-{4-[3-(4,5-dichloro-1-methyl-1H-indole-2-amido)oxetan-3-yl]-3-fluorophenyl}acetic acid;
(±)-4,5-Dichloro-N-(3-(4-(cyanomethyl)phenyl)tetrahydrofuran-3-yl)-1-methyl-1H-indole-2-carboxamide;
(±)-4-{3-[4,5-dichloro-1-methyl-6-(oxolan-3-ylmethoxy)-1H-indole-2-amido]oxolan-3-yl}benzoic acid;
(±)-4-[3-[[4,5-Dichloro-1-methyl-6-[(3-methyl-2-oxo-oxazolidin-5-yl) methoxy]indole-2-carbonyl]amino]tetrahydrofuran-3-yl]benzoic acid;
(±)-4-{3-[4,5-dichloro-1-methyl-6-(oxolan-2-ylmethoxy)-1H-indole-2-amido]oxolan-3-yl}benzoic acid;
(±)-4-(3-{4-chloro-1-methyl-6-[(2-oxo-1,3-oxazolidin-5-yl)methoxy]-1H-indole-2-amido}oxolan-3-yl)benzoic acid;
(±)-4-[3-(4-chloro-5-cyclopropyl-1-methyl-1H-indole-2-amido)oxolan-3-yl]benzoic acid;
(±)-4-(3-{4,5-dichloro-6-[2-(dimethylamino)ethoxy]-1-methyl-1H-indole-2-amido}oxolan-3-yl)benzoic acid;
(±)-4-[3-[(4,5-Dichloro-1-methyl-indole-2-carbonyl)amino]tetrahydro-furan-3-yl]-2-methyl- benzoic acid;
(±)-4-[3-[(4,5-dichloro-1-methyl-indole-2-carbonyl)amino]tetrahydro-furan-3-yl]-3-methyl- benzoic acid;
(±)-4-{3-[4,5-dichloro-6-(2-methoxyethoxy)-1-methyl-1H-indole-2-amido]oxolan-3-yl}benzoic acid;
(±)-2-cyclopropyl-4-[3-[(4,5-dichloro-1-methyl-indole-2-carbonyl)amino]-tetrahydrofuran-3-yl]benzoic acid;
(±)-4-(3-{4,5-dichloro-1-methyl-6-[(1-methyl-5-oxopyrrolidin-3-yl)methoxy]-1H-indole-2-amido}oxolan-3-yl)benzoic acid;
2-{4-[3-(4-chloro-5-methoxy-1-methyl-1H-indole-2-amido)oxetan-3-yl]phenyl}acetic acid;
(±)-4-[3-(4,5-dichloro-6-ethoxy-1-methyl-1H-indole-2-amido)oxolan-3-yl]benzoic acid;
(±)-2-[4-[3-[(4,5-dichloro-1-methyl-indole-2-carbonyl)amino]tetrahydrofuran-3-yl]phenyl]propanoic acid;
(±)-4-[3-(4-chloro-5-fluoro-1-methyl-1H-indole-2-amido)oxolan-3-yl]benzoic acid;
(±)-3-chloro-4-[3-(4,5-dichloro-1-methyl-1H-indole-2-amido)oxolan-3-yl]benzoic acid;
(R)-2-(4-(3-(4,5-dichloro-1-methyl-1H-indole-2-carboxamido) tetrahydrofuran-3-yl)phenyl)acetic acid;
(S)-2-(4-(3-(4,5-dichloro-1-methyl-1H-indole-2-carboxamido)-tetrahydrofuran-3-yl) phenyl)acetic acid;
(±)-4-[1-Acetyl-3-[(4,5-dichloro-1-methyl-indole-2-carbonyl)amino]pyrrolidin-3-yl]benzoic acid;
(±)-4-[3-[[4,5-Dichloro-1-methyl-6-[(2-oxooxazolidin-5-yl)methoxy]indole-2-carbonyl]amino]tetrahydrofuran-3-yl]benzoic acid;
2-(4-(3-(4,5-Dichloro-1-methyl-1H-indole-2-carboxamido)oxetan-3-yl)-phenyl)acetic acid;
(±)-4-(3-{9-chloro-6-methyl-2H,3H,6H-[1,4]dioxino[2,3-f]indole-7-amido}oxolan-3-yl)benzoic acid;
(±)-4-[3-(4-chloro-6-methoxy-1-methyl-1H-indole-2-amido)oxolan-3-yl]benzoic acid;
(±)-4-[3-(4,5-dichloro-6-methoxy-1-methyl-1H-indole-2-amido)oxolan-3-yl]benzoic acid;
(±)-4-[3-(4-chloro-1,5-dimethyl-1H-indole-2-amido)oxolan-3-yl]benzoic acid;
(±)-4-[3-(4-chloro-1-methyl-1H-indole-2-amido)oxolan-3-yl]benzoic acid;
(±)-4-[3-(4,5-dichloro-1-methyl-1H-indole-2-amido)oxolan-3-yl]-3-fluorobenzoic acid;
(±)-2-[3-[3-[(4,5-Dichloro-1-methyl-indole-2-carbonyl)amino]tetrahydrofuran-3-yl]phenyl]acetic acid;
(±)-2-Cyano-4-(3-(4,5-dichloro-1-methyl-1H-indole-2-carboxamido) tetrahydrofuran-3-yl)benzoic acid;
(±)-4-[3-[(4,5-Dichloro-1-methyl-indole-2-carbonyl)amino]-1-phenyl-pyrrolidin -3-yl]benzoic acid;
(±)-4-[3-(4-chloro-5-methoxy-1-methyl-1H-indole-2-amido)oxolan-3-yl]benzoic acid;
(±)-4-[3-(4,5-dichloro-1-methyl-1H-indole-2-amido)oxan-3-yl]benzoic acid 4-[4-(4,5-dichloro-1-methyl-1H-indole-2-amido)oxan-4-yl]benzoic acid;
(±)-4-[3-[(4,5-Dichloro-1-methyl-indole-2-carbonyl)amino]-1-(2,2,2-trifluoroethyl)pyrrolidin-3-yl]benzoic acid;
(±)-4-[3-[(4,5-Dichloro-1-methyl-indole-2-carbonyl)amino]-1-ethyl- pyrrolidin-3-yl]benzoic acid;
(±)-4-[3-[(4,5-Dichloro-1-methyl-indole-2-carbonyl)amino]-oxetan-3-yl]benzoic acid;
(±)-4,5-Dichloro-1-methyl-N-[3-[4-(1H-tetrazol-5-yl)phenyl]tetrahydro-furan-3-yl]indole-2-carboxamide;
(±)-2-[4-[3-[(4,5-Dichloro-1-methyl-indole-2-carbonyl)amino]tetrahydrofuran-3-yl]phenyl]acetic acid;
(±)-4-[3-[(4,5-dichloro-1-methyl-indole-2-carbonyl)amino]-1-methyl-pyrrolidin-3-yl]benzoic acid;
(±)—N-[3-(4-Carbamoylphenyl)tetrahydrofuran-3-yl]-4,5-dichloro-1-methyl-indole-2-carboxamide;
(R)-4-[(3)-3-[(4,5-dichloro-1-methyl-indole-2-carbonyl)-amino]-tetrahydrofuran-3-yl]benzoic acid;
(S)-4-[(3)-3-[(4,5-Dichloro-1-methyl-indole-2-carbonyl)amino]-tetrahydrofuran -3-yl]benzoic acid;
(±)-4,5-dichloro-N-[3-(4-cyanophenyl)tetrahydrofuran-3-yl]-1-methyl-indole-2-carboxamide;
(±)—N-[3-(4-Bromophenyl)tetrahydrofuran-3-yl]-4,5-dichloro-1-methyl-indole-2-carboxamide;
(±)-4-[3-[(4,5-Dichloro-1-methyl-indole-2-carbonyl)amino]tetrahydrofuran-3-yl]benzoic acid;
(±)-4-[3-[(4,5-Dichloro-1-methyl-indole-2-carbonyl)amino]tetrahydrofuran-3-yl]benzoic acid;
(±)-4,5-Dichloro-N-[3-[3-(hydroxymethyl)phenyl]-1-(methylcarbamoyl-sulfamoyl)-3-piperidyl]-1-methyl-indole-2-carboxamide;
(±)-3-[3-[(4,5-Dichloro-1-methyl-indole-2-carbonyl)amino]-1-(methylcarbamoylsulfamoyl)-3-piperidyl]benzoic acid;
(±)-4-(3-(4,5-Dichloro-1-methyl-1H-indole-2-carboxamido)-1-(N-(methylcarbamoyl) sulfamoyl)piperidin-3-yl)benzoic acid;
(±)-N-[3-(3-Carbamoylphenyl)tetrahydrofuran-3-yl]-4,5-dichloro-1-methyl-indole-2-carboxamide;
(±)-4,5-Dichloro-N-[3-(3-cyanophenyl)-1-(methylcarbamoylsulfamoyl)-3-piperidyl]-1-methyl-indole-2-carboxamide;
(±)-Dichloro-N-[3-(3-cyanophenyl)tetrahydrofuran-3-yl]-1-methyl-indole-2-carboxamide;
4-[1-(Acetylsulfamoyl)-4-[(4,5-dichloro-1-methyl-indole-2-carbonyl)amino]-4-piperidyl]benzoic acid;
N-(1-(N-Acetylsulfamoyl)-4-(3-aminophenyl)piperidin-4-yl)-4,5-dichloro-1-methyl-1H-indole-2-carboxamide;
(±)-4,5-dichloro-1-methyl-N-[1-(methylcarbamoylsulfamoyl)-3-phenyl-3-piperidyl]indole-2-carboxamide;
(±)-4,5-Dichloro-N-[3-(3-cyanophenyl)-1-(methylcarbamoylsulfamoyl)-3-piperidyl]-1-methyl-indole-2-carboxamide;
N-(1-(N-acetylsulfamoyl)-3-phenylazetidin-3-yl)-4,5-dichloro-1-methyl-1H-indole-2-carboxamide;
N-(1-(N-acetylsulfamoyl)-4-(3-(hydroxymethyl)phenyl)piperidin-4-yl)-4,5-dichloro -1-methyl-1H-indole-2-carboxamide;
N-(4-(3-Acetamidophenyl)-1-acetylpiperidin-4-yl)-4,5-dichloro-1-methyl-1H-indole-2-carboxamide;
3-(1-(N-acetylsulfamoyl)-4-(4,5-dichloro-1-methyl-1H-indole-2-carbox-amido) piperidin-4-yl)benzoic acid;
4,5-Dichloro-1-methyl-N-[4-phenyl-1-(pyrazin-2-ylsulfamoyl)-4-piperidyl]indole-2-carboxamide;
N-(4-(3-acetamidophenyl)-1-(N-acetylsulfamoyl)piperidin-4-yl)-4,5-dichloro-1-methyl-1H-indole-2-carboxamide;
N-(1-(N-acetylsulfamoyl)-4-(3-cyanophenyl)piperidin-4-yl)-4,5-dichloro-1-methyl-1H-indole-2-carboxamide;
N-(1-(N-acetylsulamoyl)-4-phenylpiperidin-4-yl)-4,5-dichloro-1-methyl-1H-indole-2-carboxamide;
(±)-ethoxy-1-methyl-1H-indole-2-amido)oxetan-3-yl]phenyl}-2-cyclohexylacetic acid;
2R)-2-[4-[3-[(4-chloro-5-methoxy-1-methyl-indole-2-carbonyl)amino]oxetan-3-yl]phenyl]-3-methyl-butanoic acid;
(2S)-2-[4-[3-[(4-chloro-5-methoxy-1-methyl-indole-2-carbonyl)amino]oxetan-3-yl]phenyl]-3-methyl-butanoic acid;
(±)-2-{4-[3-(4-chloro-5-methoxy-1-methyl-1H-indole-2-amido)oxetan-3-yl]phenyl}-2-(piperidin-4-yl)acetic acid;
(±)-2-[4-[3-[(4-Chloro-5-methoxy-1-methyl-indole-2-carbonyl)amino]oxetan-3-yl]phenyl]-2-(4-hydroxycyclohexyl)acetic acid;
4-Chloro-5-methoxy-1-methyl-N-(3-(4-(N-propionylsulfamoyl)phenyl)-oxetan-3-yl)-1H-indole-2-carboxamide;
4-Chloro-5-methoxy-1-methyl-N-(3-(4-((methylsulfonyl)carbamoyl)-phenyl)oxetan-3-yl)-1H-indole-2-carboxamide;
(±)-2-Cyclopropyl-2-(4-(3-(4,5-dichloro-6-methoxy-1-methyl-1H-indole-2-carboxamido) tetrahydrofuran-3-yl)phenyl)acetic acid;
4-chloro-N-{3-[4-(acetamidosulfonyl)phenyl]oxetan-3-yl}-5-methoxy-1-methyl-1H-indole-2-carboxamide;
(±)-2-{4-[3-(4-chloro-5-methoxy-1-methyl-1H-indole-2-amido)oxetan-3-yl]phenyl}-2-(oxan-4-yl)acetic acid;
as single deuterates, enantiomers, diastereoisomers or mixtures thereof, in any proportion.

14: The method according to claim 13, wherein the compound is

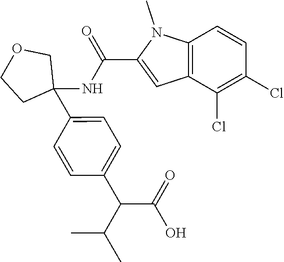 [SMILES: CC(C)C(C(=O)O)c1ccc(C2(NC(=O)c3cc4c(Cl)c(Cl)ccc4n3C)CCOC2)cc1]

15: The method according to claim 13, wherein the compound is diasteroisomer 2 of (±)-2-(4-(3-(4,5-Dichloro-1-methyl-1H-indole-2-carboxamido)tetrahydro-furan-3-yl) phenyl)-3-methylbutanoic acid.

16: The method according to claim 1, wherein the fibrosis is at least one selected from the group consisting of pulmonary fibrosis, idiopathic pulmonary fibrosis (IPF), chronic hypersensitivity pneumonitis (CHP), hepatic fibrosis, kidney or renal fibrosis, ocular fibrosis, cardiac fibrosis, arterial fibrosis, sarcoidosis and systemic sclerosis, liver fibrosis and Cirrhosis.

17: The method according to claim 16 wherein the fibrosis comprises IPF.

18: The method according to claim 1, wherein the compound of Formula (I) or pharmaceutically acceptable salt thereof is present in a pharmaceutical composition in admixture with one or more pharmaceutically acceptable carriers or excipients.

19: The method according to claim 18 wherein the fibrosis is selected from the group consisting of pulmonary fibrosis, idiopathic pulmonary fibrosis (IPF), chronic hypersensitivity pneumonitis (CHP), hepatic fibrosis, kidney or renal fibrosis, ocular fibrosis, cardiac fibrosis, arterial fibrosis, sarcoidosis and systemic sclerosis, liver fibrosis and Cirrhosis.

20: The method according to claim 19 wherein the fibrosis comprises IPF.

21: The method according to claim 19, comprising oral administration of the pharmaceutical composition.
